(19) United States
(12) Patent Application Publication
(10) Pub. No.: US 2025/0302330 A1
(43) Pub. Date: Oct. 2, 2025

(54) CURVE INDUCTIVE SENSOR

(71) Applicant: Magnisity Ltd., Hod-HaSharon (IL)

(72) Inventors: Ron BARAK, Tel-Aviv (IL); Benjamin GREENBURG, Hod-HaSharon (IL); Dror GARDOSH, Rishon LeTsiyon (IL); Eyal KLEIN, Tel-Aviv (IL)

(73) Assignee: Magnisity Ltd., Hod-HaSharon (IL)

(21) Appl. No.: 18/864,927

(22) PCT Filed: May 11, 2023
(86) PCT No.: PCT/IL2023/050493

§ 371 Date: Nov. 12, 2024

Related U.S. Application Data
(63) Continuation of application No. PCT/IL2022/051241, filed on Nov. 21, 2022.
Application No. PCT/IL2022/051241 is a continuation of application No. PCT/IL2022/051242, filed on Nov. 21, 2022.
(60) Provisional application No. 63/281,686, filed on Nov. 21, 2021.
(60) Provisional application No. 63/281,686, filed on Nov. 21, 2021.
(60) Provisional application No. 63/341,046, filed on May 12, 2022.
(60) Provisional application No. 63/341,062, filed on May 12, 2022.
(60) Provisional application No. 63/341,062, filed on May 12, 2022.
(60) Provisional application No. 63/341,062, filed on May 12, 2022.
(60) Provisional application No. 63/406,787, filed on Sep. 15, 2022.
(60) Provisional application No. 63/406,787, filed on Sep. 15, 2022.
(60) Provisional application No. 63/406,787, filed on Sep. 15, 2022.

(51) Int. Cl. A61B 5/06; A61B 5/00

(52) U.S. Cl. CPC A61B 5/066; A61B 5/742

(57) ABSTRACT

Flexible, longitudinally extended sensors comprising variably resistive and/or inductive sensing material. Sensors connect to an external processing unit using as few as 2 wires. In operation, sensors measure resistance/inductance along their longitudinal extent (curve). Spectral de-multiplexing allows electrical property differences along sensors to be resolved. This enables shape tracking of the flexible sensor. The sensor principles are suitable for the production of ultra-small diameter probes (for example, smaller than 0.5 mm) of arbitrary length (for example, 30 cm long). There are potential advantages for cost efficiency and ease of manufacture. The sensor can be constructed as a shape tracked guidewire to enable endovascular navigation procedures. The sensor's tracked shape enables deformation tracking of the anatomy. Using the sensor's tracked shape rather than repetitive X-ray imaging improves navigational accuracy and reduces exposure to radiation.

### RELATED APPLICATION/S

This application claims the benefit of priority of U.S. Provisional Patent Application No. 63/341,062 filed May 12, 2022; U.S. Provisional Patent Application No. 63/341,046 filed on May 12, 2022 and of U.S. Provisional Patent Application No. 63/406,787 filed Sep. 15, 2022; PCT Patent Application No. PCT/IL2022/051241, filed on Nov. 21, 2022 and PCT Patent Application No. PCT/IL2022/051242, filed on Nov. 21, 2022, the contents of which are incorporated herein by reference in their entirety.

This application is also related to U.S. Provisional Patent Application No. 63/281,686 filed Nov. 21, 2021. The contents of the above applications are all incorporated by reference as if fully set forth herein in their entirety.

### FIELD AND BACKGROUND OF THE INVENTION

The present invention, in some embodiments thereof, relates to the field of microprobe position and/or shape sensing and more particularly, but not exclusively, to sensing of probe positions and/or shapes using electrical field measurements.

Certain physical quantities can be measured by measuring the electrical resistance of a conductor affected by the physical phenomenon. For example, a resistance thermometer comprises a material which has an accurate resistance/temperature relationship which is used to provide indication of the temperature. In a strain gauge, the strain of an object is computed by measuring the electrical resistance of a foil attached to an object. As the object is deformed, the electrical resistance of the deformed foil changes which provides indication of the strain. Similarly, a force/pressure sensor uses a force-sensitive resistor to measure the force applied to the sensor. A magnetoresistive sensor comprises a foil (for example, permalloy, supermalloy, mu-metal, or cobalt alloy) which changes its resistance due to an externally applied magnetic field. The magnetoresistive sensor measures the resistance of the foil to compute the magnetic field at the position and orientation of the sensor in space by using a known resistance/magnetic field relationship.

A magneto-inductive sensor measures the inductance of a coil wrapped around a high permeability non-linear magnetic core (such as permalloy, supermalloy, mu-metal etc.) to compute the magnetic field at the position and orientation of the sensor in space by using a known inductance/magnetic field relationship.

To measure the electrical resistance of a conductor to a sufficient precision to detect and quantify variable resistive effects, a Wheatstone bridge is commonly used. The Wheatstone bridge converts the electrical resistance to be measured into a differential voltage quantity which can then be amplified, filtered and sampled with an ADC (Analog Digital Converter). A precisely known relation between the measured electrical resistance and the physical quantity (e.g., temperature, strain, force/pressure, magnetic field strength) is used in order to convert the measured resistance into a measurement of the desired physical quantity.

Several methods exist for measuring the inductance of an inductor to a precision sufficient to detect magneto-inductive changes. Some inductors have rather constant inductance L of some range of currents and frequencies, and can be measured for example using oscillation-based methods: for example, the inductor can be placed in a known RLC-type circuit and the resonance frequency f can be measured, which depends on the inductance of the inductor (as well as on values of R and C, which may be accounted for as known values). By knowing the frequency to inductance's (f-L) exact relationship, the inductance can be solved. A precisely known relation between the measured electrical resistance or inductance and the physical quantity (e.g., temperature, strain, force/pressure, or magnetic field) is used in order to convert the measured resistance or inductance into a measurement of the desired physical quantity.

In U.S. Pat. No. 9,658,298, a 3-axis magnetoresistive sensor is described which senses the magnetic field along 3 non-coplanar axes. The sensed quantities are then converted into a full 3-dimensional magnetic field measurement at the position and orientation of the sensor by applying calibration matrices, to convert the field measured in potentially non-orthogonal axes and non-unity gains to the sensor's orthonormal axes with unity gains.

In U.S. Pat. Publication No. 2013/0009635 A1 a magneto-inductive sensor is described which senses the magnetic field along magneto-inductive coils. The sensor measures the inductance of discrete coils, wrapped around a high permeability magnetic core, and converts the measured inductances to magnetic field measurements. The inductance is measured using digital oscillation techniques.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A method of displaying a navigational view for an endoluminal device, comprising: a. receiving at least one 2-D image comprising a lumen to be navigated by said endoluminal device;
b. detecting a 3-D location of a tip and a shape of said endoluminal device;
c. calculating a position and/or a shape of said endoluminal device within said at least one 2-D image;
d. displaying on said 2-D image said endoluminal device according to said calculation;
e. amending said displaying of said endoluminal device on said 2-D image by repeating steps “b” and “c” while said endoluminal device is being moved.

Example 2. The method according to example 1, wherein said method does not require retaking new images in order to perform said “e”.

Example 3. The method according to example 1, wherein said at least one 2-D image is a 2-D X-ray image.

Example 4. The method according to example 1, wherein said at least one 2-D image comprises within a 2-D view of at least one segment of said endoluminal device.

Example 5. The method according to example 4, wherein said calculating a position and/or a shape of said endoluminal device comprises comparing said at least one segment as viewed in said at least one 2-D image with said detected 3-D location of said tip and said shape of said endoluminal device in order to identify a location of said at least one segment along said endoluminal device.

Example 6. The method according to example 5, further comprising utilizing said identified location to perform a comparison between said detected 3-D location of said tip and said shape of said endoluminal device and said at least one 2-D image.

Example 7. The method according to example 6, further comprising analyzing said comparison to generate a navigational view of said endoluminal device on said at least one 2-D image.

Example 8. The method according to example 7, further comprising displaying said generated navigational view by re-projecting a result of said analysis on said at least one 2-D image.

Example 9. The method according to example 8, further comprising amending said displaying of said generated navigational view by repeating examples 5-8 while said endoluminal device is being moved.

Example 10. The method according to example 9, wherein said amending does not require retaking new images in order to perform said amending.

Example 11. The method according to example 1, wherein said at least one 2-D image comprises, within said at least one 2-D image, a 2-D view of one or more EM markers and/or EM reference sensors located at EM known locations.

Example 12. The method according to example 11, further comprising correlating said known 3-D location of said one or more EM markers and/or EM reference sensors with a location of said one or more EM markers and/or EM reference sensors in said 2-D image.

Example 13. The method according to example 12, further comprising comparing said correlation performed in example 12 with said detected 3-D location of said tip and said shape of said endoluminal device.

Example 14. The method according to example 13, further comprising analyzing said comparison to generate a navigational view of said endoluminal device on said at least one 2-D image.

Example 15. The method according to example 14, further comprising displaying said generated navigational view by re-projecting a result of said analysis on said at least one 2-D image.

Example 16. The method according to example 1, wherein said at least one 2-D image comprises a 2-D view of a plurality of markers located in known locations along said endoluminal device.

Example 17. The method according to example 16, further comprising comparing a location of said plurality of markers located in known locations along said endoluminal device as viewed in said at least one 2-D image with their actual 3-D known location along said endoluminal device and according to said detected 3-D location of said tip and said shape of said endoluminal device.

Example 18. The method according to example 17, further comprising analyzing said comparison to generate a navigational view of said endoluminal device on said at least one 2-D image.

Example 19. The method according to example 18, further comprising displaying said generated navigational view by re-projecting a result of said analysis on said at least one 2-D image.

Example 20. The method according to example 19, further comprising amending said displaying of said generated navigational view by repeating examples 12-14 while said endoluminal device is being moved.

Example 21. The method according to example 20, wherein said amending does not require retaking new images in order to perform said amending.

Example 22. The method according to example 1, wherein said detecting a 3-D location of a tip and a shape of said endoluminal device is performed utilizing one or more of an EM inductive sensor and an EM resistive sensor.

Example 23. The method according to example 1, wherein said detecting a 3-D location of a tip and a shape of said endoluminal device is performed by one or more of EM tip sensing (for example, single-coil EM sensor), multi-sensor EM shape sensing (using multiple individual EM sensors to sense the shape of the device), fiber optic shape sensing, passive RF sensing, sensing detectable magnets, sensing ultrasound-detectable markers and fluoroscopic shape tracking.

Example 24. The method according to example 1, wherein said displaying comprises displaying on a 3-D roadmap said endoluminal device according to said calculation.

Example 25. The method according to example 24, further comprising amending said displaying of said endoluminal device on said 3-D roadmap by repeating steps “b” and “c” while said endoluminal device is being moved.

Example 26. The method according to example 11, further comprising utilizing said EM reference sensors to track a movement of a patient; and said method further comprises compensating for said movements performed by said patient by moving said detected 3-D location of said tip and said shape of said endoluminal device accordingly.

Example 27. A method of displaying a navigational view for an endoluminal device, comprising:
a. receiving at least one 3-D roadmap comprising a lumen to be navigated by said endoluminal device;
b. detecting a 3-D location of a tip and a shape of said endoluminal device;
c. calculating a position and/or a shape of said endoluminal device within at least one 3-D image of said 3-D roadmap;
d. displaying on said 3-D image said endoluminal device according to said calculation;
e. amending said displaying of said endoluminal device on said 3-D image by repeating steps “b” and “c” while said endoluminal device is being moved.

Example 28. The method according to example 27, wherein said method does not require retaking new 3-D roadmaps and/or 3-D images and/or 2-D images in order to perform said “e”.

Example 29. The method according to example 27, further comprising deforming said 3-D roadmap based on said detected tip and shape of said endoluminal device; and displaying a navigational view using said deformed 3-D roadmap and said detected tip and shape of said endoluminal device.

Example 30. The method according to example 27, wherein said displaying is performed on a 2-D image.

Example 31. A method of displaying a navigational view for an endoluminal device, comprising:
a. receiving at least one 2-D image including, within said at least one 2-D image, a 2-D view of a plurality of markers located in known locations along said endoluminal device;
b. detecting a 3-D location of a tip and a shape of said endoluminal device;
c. comparing a location of said plurality of markers located in known locations along said endoluminal device as viewed in said at least one 2-D image with their actual 3-D known location along said endoluminal device and according to said detected 3-D location of said tip and said shape of said endoluminal device;
d. analyzing said comparison to generate a navigational view of said endoluminal device on said at least one 2-D image;
e. displaying said generated navigational view by re-projecting a result of said analysis on said at least one 2-D image.

Example 32. The method according to example 31, wherein said method does not require retaking new images in order to generate said displaying.

Example 33. The method according to example 31, wherein said at least one 2-D image is a 2-D X-ray image.

Example 34. A method of displaying a navigational view for a field of view for an endoluminal device, comprising:
a. receiving at least one 2-D image including, within said at least one 2-D image, a 2-D view of at least one segment of said endoluminal device;
b. detecting a 3-D location of a tip and a shape of said endoluminal device;
c. comparing said at least one segment as viewed in said at least one 2-D image with said detected 3-D location of said tip and said shape of said endoluminal device in order to identify a location of said at least one segment along said endoluminal device;
d. utilizing said identified location to perform a comparison between said detected 3-D location of said tip and said shape of said endoluminal device and said at least one 2-D image;
e. analyzing said comparison to generate a navigational view of said endoluminal device on said at least one 2-D image;
f. displaying said generated navigational view by re-projecting a result of said analysis on said at least one 2-D image.

Example 35. The method according to example 34, wherein said method does not require retaking new images in order to generate said displaying.

Example 36. The method according to example 34, wherein said at least one 2-D image is a 2-D X-ray image.

Example 37. A method of displaying a navigational view for a field of view for an endoluminal device, comprising:
a. receiving at least one 2-D image including, within said at least one 2-D image, a 2-D view of one or more EM markers located at EM known locations;
b. correlating said known location of said one or more EM markers with a location of said one or more markers in said 2-D image;
c. detecting a 3-D location of a tip and a shape of said endoluminal device;
d. comparing said correlation performed in “b” with said detected 3-D location of said tip and said shape of said endoluminal device;
e. analyzing said comparison to generate a navigational view of said endoluminal device on said at least one 2-D image;
f. displaying said generated navigational view by re-projecting a result of said analysis on said at least one 2-D image.

Example 38. The method according to example 37, wherein said method does not require retaking new images in order to generate said displaying.

Example 39. The method according to example 37, wherein said at least one 2-D image is a 2-D X-ray image.

Example 40. A method of displaying a navigational view for a field of view for an endoluminal device, comprising:
a. generating a volume from a plurality of images;
b. detecting tip and shape of said endoluminal device;
c. deforming said volume based on said detected tip and shape of said endoluminal device; and
d. displaying a navigational view using said deformed volume and said detected tip and shape of said endoluminal device.

Example 41. The method according to example 40, wherein said generating a volume from a plurality of images comprises one or more of:
a. receiving said plurality of images;
b. analyzing said plurality of images to detect one or more vessels within said plurality of images;
c. combining multiple phases of said detected one or more vessels into a single data structure comprising vessels of interest in said field of view;
d. combining results from “b” and “c” into a common 3-D space, thereby generating said volume.

Example 42. The method according to example 41, wherein said combining results from “b” and “c” into a common 3-D space, further comprises combining vascular segments with their associated 3-D spatial extends.

Example 43. The method according to example 40, wherein said plurality of images are one or more of angiograms images, X-ray images, Cone-beam images, CT images, MRI images.

Example 44. The method according to example 40, wherein said volume comprises one or more data comprising descriptions of paths along which vascular centerlines extend, descriptions of nodes at which paths join and/or bifurcate, and descriptions of vascular cross-sections along the paths.

Example 45. The method according to example 40, wherein said generating a volume from a plurality of images further comprises associating said generated volume with a deformation model.

Example 46. The method according to example 41, wherein said receiving said plurality of images is performed in real-time.

Example 47. The method according to example 40, wherein said detecting tip and shape of said endoluminal device comprises one or more of:
a. associating between said endoluminal device and sensor raw data received from one or more sensors in said endoluminal device;
b. reconstructing a 3-D shape of said endoluminal device based on said association; and
c. detecting a shape location of said endoluminal device based on a coordinate system.

Example 48. The method according to example 47, wherein said one or more sensors comprise one or more of a inductive EM sensor and a resistive EM sensor.

Example 49. The method according to example 47, wherein said deforming said volume based on said detected tip and shape of said endoluminal device comprises one or more of:
a. calculating said deforming based on constrains imposed by said reconstructing and said detecting of example 24; and
b. calculating said deforming based on received images taken in real-time.

Example 50. The method according to example 40, wherein said displaying is performed on a 2-D X-ray image.

Example 51. The method according to example 40, wherein said endoluminal device comprises one or more radiopaque markers; and wherein said method further comprises utilizing said one or more markers for generating said displaying by correlating a position of said one or more radiopaque markers in relation to a position of said endoluminal device as received by sensors within said endoluminal device with said plurality of images.

Example 52. The method according to example 40, wherein said “c” and said “d” are performed multiple times.

Example 53. The method according to example 40, wherein said displaying comprises displaying one or more of:
a. a deformed 3-D model of said field of view; and
b. a shape of said endoluminal device with respect of said deformed 3-D model.

Example 54. The method according to example 40, wherein said displaying comprises displaying in a virtual reality and/or augmented reality display and/or augmented fused display (such as 3-D tracked device reprojected on 2-D X-ray image).

Example 55. The method according to example 40, wherein said displaying comprises displaying said tip of said endoluminal device in relation to a chosen point of view.

Example 56. The method according to example 40, further comprising modifying said field of view according to a position of said tip of said endoluminal device.

Example 57. The method according to example 40, wherein said displaying does not require retaking new images in order to generate said displaying.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Instruction executing elements of the processor may comprise, for example, one or more microprocessor chips, ASICs, and/or FPGAs. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. Additionally or alternatively, sequences of logical operations (optionally logical operations corresponding to computer instructions) may be embedded in the design of an ASIC and/or in the configuration of an FPGA device. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus such as an FPGA, or other devices such as ASICs to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 schematically illustrates-wire sensor, with electrical properties divided to discrete units; either actually, or for purposes of description and analysis; according to some embodiments of the present disclosure;

FIG. 2 shows an example of a total impedance vs. frequency relation of a sensor with eight resistive elements and reactive components, according to some embodiments of the present disclosure;

FIG. 3 schematically illustrates-wire sensor, with electrical properties divided to discrete (but infinitesimal) units for purposes of description and analysis, according to some embodiments of the present disclosure;

FIG. 4 is a schematic flowchart describing a method of determining the position and shape of a flexible sensor within a set of generated magnetic fields, according to some embodiments of the present disclosure;

FIG. 5 schematically represents an endovascular tracking system, according to some embodiments of the present disclosure;

FIG. 6 schematically represents a two layer sensor FPC with discrete “barber pole” resistive elements, according to some embodiments of the present disclosure;

FIG. 7 schematically illustrates a twisted-pair variable resistive sensor configuration, according to some embodiments of the present disclosure;

FIG. 8 plots a magnetization to inductance relationship of an example magneto-inductive coil, wrapped around a high permeability non-linear magnetic core, according to some embodiments of the present disclosure;

FIG. 9 schematically represents an inductive sensor comprising discrete coils connected in series, according to some embodiments of the present disclosure;

FIG. 10 schematically represents a flexible sensor comprising a single coil made of discrete coil elements connected in series, according to some embodiments of the present disclosure;

FIG. 11 schematically represents a flexible sensor comprising a coil with continuously decreasing winding pitch, according to some embodiments of the present disclosure;

FIG. 12A plots a magnetization to inductance relationship of an example magneto-inductive coil with varying pitch, under an externally applied magnetic field which varies along the sensor's curve, according to some embodiments of the present disclosure;

FIG. 12B schematically indicates features of data acquired using a magneto-inductive sensor according to some embodiments of the present disclosure;

FIG. 13 schematically represents a guidewire having an integrated flexible sensor comprising a coil wound around a core which is constructed as a distal extension of the main guidewire body, according to some embodiments of the present disclosure;

FIG. 14A schematically represents an endovascular tracking system, according to some embodiments of the present disclosure;

FIG. 14B schematically diagrams operations of a system for tracking of angiogram deformation and probe position using curve inductive sensor, according to some embodiment of the present disclosure;

FIGS. 15A-15B schematically represent an acquired visualization of a lumen anatomy, with and without deformation compensation, according to some embodiment of the present disclosure;

FIG. 16 schematically represents an angiogram-like navigation screen using a-D deformed model of luminal anatomy, according to some embodiment of the present disclosure; and

FIGS. 17A-17C schematically represent “first-person” views of navigation in a-D model, wherein the view represents what lies in front of the tip of an endoluminal device, according to some embodiment of the present disclosure.

### DESCRIPTION OF SPECIFIC EMBODIMENTS OF THE INVENTION

The present invention, in some embodiments thereof, relates to the field of microprobe position and/or shape sensing and more particularly, but not exclusively, to sensing of probe positions and/or shapes using electrical field measurements.

### Overview

A broad aspect of some embodiments of the present disclosure relates to multiplexed, spatially resolved sensing of environmentally induced local effects upon a sensing device and/or material. In some embodiments, the sensing device and/or material is provided as a flexible and elongated sensor (i.e., long and thin). The linearly elongated sensor may be provided more particularly as part of (e.g., at a distal end of) a medical device probe, for example, a guide-wire, catheter-delivered tool, or catheter portion. The medical device probe may be configured for endoluminal navigation; e.g., for endobronchial and/or endovascular navigation, and/or specifically for neurovascular navigation and/or lymph system navigation.

In some embodiments, a purpose of the navigation is to bring and/or help guide a treatment, sampling, and/or diagnostic tool to a target of treatment, sampling, and/or diagnosis. In some embodiments, a purpose of the navigation is to reach another device such as a previously implanted device, for example to configure it, maintain it, and/or evaluate its functioning.

In some embodiments, an environmental parameter that is sensed by causing the local effects is local magnetic field strength and/or direction. Herein, these are also referred to together as the local magnetic field vector. Other definitions related to magnetic field properties are provided hereinbelow.

Spatially resolved measurement may rely, for example, on a sensing material having properties such as magnetoresistance (change in electrical resistance as a function of surrounding electromagnetic field influences), or on a sensing device comprising a coil having a core material with permeability that is non-linearly affected by magnetic fields it is exposed to. In some embodiments, environmentally-responsive inductance (non-linear permeability) and resistance properties are both provided over a spatial extent of a device, for example to provide separate measurement sources, to enhance each other's selectivity, and/or to assist calibration and/or compensation for secondary effects of the environment and/or intrinsic properties of the sensing circuit.

An aspect of some embodiments of the present disclosure relates to elongate probes which provide position sensing with respect to magnetic fields.

In some embodiments, a sensing region of the elongate probe is spanned by an electrical conductor (optionally a uniform wire or comprised a plurality of serially interconnected electrical conductors) which interconnects on first and second ends to a readout controller. The readout controller passes current into the electrical conductor, and measures electrical signals produced as a result. The signal itself integrates properties from all along the electrical conductor. Structurally, the sensing region is comprised of regions along its length which differ in their structure, and more particularly in an effect that this structure has on electrical current flowing through it, which is indicated in the measurements of the electrical signals. Furthermore, the sensing region is structured to produce this effect differently, depending on the magnetic field it is exposed to (e.g., local magnetic field strength).

The raw measurements from the electrical signals conflate these effects, producing total measurements, e.g., totaling the effects on the current produced everywhere along the electrical conductor.

In some embodiments, a processor decomposes the effects and assigns them, in a form such as an estimate of local magnetic field strength, to individual locations along the sensing probe. To do this, the processor uses the measurements themselves, along with models of the conditions under which the measurements were taken. In particular, expected responses to local magnetic field and the structural differences (at least their electrical effects) are modeled.

Furthermore, in some embodiments, a model of magnetic field distribution in a region containing the sensing region is used to determine where the sensing probe is, and/or its shape. For example, sensing region locations with their respective estimated local magnetic field strengths are assigned positions and/or a shape in 3-D spatial coordinates which consistently match the model of magnetic field distribution.

An aspect of some embodiments of the present disclosure relates to the use for shape determination and/or position finding of devices having electrical induction properties (e.g., core material electromagnetic field permeability) which experience spatially localized alterations induced by interactions with the environment. In some embodiments, the device is constructed so that the measurements can be spectrally multiplexed, allowing determination of which parts of the device are affected by external EM fields, and to what extent.

In some embodiments of the present disclosure, a sensor device comprises a continuous long and narrow inductive element (for example, 0.3 mm in diameter and 30 cm long coil). Alternatively, in some embodiments, a set of discrete inductive elements connected in series is provided.

However configured, inductor coils may be wrapped around a high permeability non-linear magnetic core; made, for example, of permalloy, supermalloy, mu-metal, cobalt alloy, or any other high permeability non-linear magnetic core. Relative permeability of a material is expressed as a dimensionless number. Permeability may be handled as complex number particularly in high-frequency applications. Examples of (real number) maximum relative permeability values for these materials include, for example: 100000 for permalloy, 800000 for supermalloy, 100000 for mu-metal, and 100000 for some cobalt-based alloys.

The non-linearity comprises changes in the permeability of the core material as it is exposed to electromagnetic fields having different properties (e.g., different intensities and/or frequencies). The core material may be intrinsically flexible; e.g., flexible when provided as a straight wire, as a coil, or as a braided or other compound structure. Additionally or alternatively, the extent of the core material may be given flexibility by coupling to and/or blending with another material. For example, in some embodiments core material is segmented, and the segments supported using another material such as a polymer coating or flexible strands (e.g., wires). In some embodiments, the high permeability non-linear magnetic core material is blended with a flexible matrix material. Thus configured, the flexible sensor is linear; that is, much longer than its diameter, and unbranched between two ends. The ratio of length to diameter is, for example, at least 20, and preferably at least a larger factor of 50 or 100. The maximum diameter, in some embodiments, is less than about 5 mm, and equal to or less than about 2 mm, about 1 mm, about 0.5 mm, or about 0.36 mm. In particular, reaching a diameter at least as small as 0.36 mm (0.014 inches) provides a potential advantage by being small enough for use as a neurovascular probe, and/or for use as a probe through lumens of the lymph system.

For use in shape determination and/or position finding, the sensor may be used alone, or coupled to another device; for example, embedded inside a catheter or an endoscope. In some embodiments, the sensor itself is further configured as a device with additional features as a use case may call for. For example, the core material may itself be formed to provide the lumenal wall of a microcatheter, and/or with the mechanical properties (e.g., steerability, torqueability, and/or pushability) appropriate to a microcatheter or guidewire, or portion thereof. For example, it may be pushable, as the distal end of such a device, to a distance of at least 50 mm, into a body cavity region having an inner diameter of 5 mm or less. Optionally, at least a distal tip of the device is pushable into a body cavity region of at least 1 mm or less, 0.5 mm or less, or about 360 μm or less.

Suitable readout and processing of data measured from the sensor potentially provides real-time position and/or shape tracking of the catheter or other device so-equipped, for example, full shape tracking in 3-D. Except as otherwise indicated, “real-time” tracking comprises updating shape and/or position at least once per second; or more often, for example, a frequency of at least 5 Hz, 10 Hz, or 30 Hz. Updates may be used for system-internal purposes, e.g., in automated tracking, and/or may be provided as displays or other indications to device operators. Potential advantages of providing real-time tracking displays/indications to an operator include rapid feedback which allows adjusting control inputs to achieve intended movements of a device, and assisting in developing and/or maintaining for the operator a sense of the manipulated device as a “real” device, e.g., a unified impression of the state of the device, although based on indirect and/or artificially constructed measurements, and potentially measurements obtained through a plurality of indirect and/or artificial sensory modalities. Potentially, providing real-time feedback helps the operator learn to associate certain inputs to the device with certain likely results.

One manner of using a plurality of discrete coils is to connect wire pairs extending from coil ends to an external processing unit (readout controller) as multiple twisted pairs. The processing unit would measure the inductance of each discrete coil independently, for example using oscillation techniques or any other suitable method, to provide multiple inductance measurements which can be used as indications of the desired physical quantity or quantities. For example, the physical quantities may comprise magnetic field properties, and these may in turn be related to spatial positions having (or supposed to have) those particular magnetic field properties. However, in this configuration the number of wires may increase linearly with the number of coils along the sensor, reducing practicality for applications where small footprint is crucial.

In some embodiments of the present disclosure, an inductor comprising a single long coil (or set of coils in series) is provided, connected by its ends to an external processing unit. The processing unit measures total inductance of the inductor, which amounts to the line integral of the inductance density (i.e., the inductance per unit length) along the sensor's curve.

Under appropriate field conditions, the resulting single time-series measurement of total inductance along the sensor's curve, potentially contains information suitable for resolving information indicative of the sensor's shape and/or position. For example, in some embodiments, an EM field generator is provided which transmits a large number of spatially distinct AC fields, each at a different frequency. For example, 30 different fields may be transmitted through the vicinity of the sensor. The mean inductance measured using a single magneto-inductive coil can be used to extract 30 values from data sampled over a fairly short time period (for example, in a 30 millisecond window). Analysis for the extraction may, for example, use DFT (Discrete Fourier Transform) methods, correlation methods, or another suitable method. Together with knowledge and/or assumptions about the spatial distribution of the various EM fields being produced, these values are indicative of the sensor's full shape in space, relative to the transmitter. Under certain smoothness constraints, location constraints, distance constraints, and/or flexibility constraints applicable to the construction of the shape sensor, the sensor's full shape can be solved to a good accuracy based on the 30 sensed values. Herein, “smoothness constraints” refer constraints which encourage or require a model parameter representing a physical parameter such as magnetic field strength be continuously variable over space and/or time (i.e., not discontinuous). “Location constraints” refer to constraints on a model parameter representing sensed or a priori knowledge (complete and/or partial) of where an element is. For example, a sensor portion may be constrained to within a lumenal cavity of a body, e.g., insofar as it is known to have been introduced to that lumen, and assumed not to have punctured the lumenal cavity wall. “Distance constraints” refer to constraints on a model parameter representing a constant size or a size that changes within certain limits. For example, a known distance between two locations on a probe may be used as a distance constraint. “Flexibility constraints” refer to constraints on a model parameter representing limitations on how much a structure may bend. For example, a flexible probe may be modeled with an absolute limitation on its minimum radius of curvature, and/or curvature may be penalized within a certain range of curvature radii. Constraints may be implemented, e.g., in the form of absolute constraints, and/or in a form that affects likelihood. For example, they may be implemented in the form of penalties, such as penalties applied as errors during a computational process of error minimization to find a physical model state that explains sensed data.

Due to footprint constraints in several applications, specifically in medical applications, it is a potential advantage to connect as few wires as possible to the shape sensor. Since a shape sensor comprising a single magneto-inductive coil as described above may involve the use of an unusual and/or expensive and/or impractical EM field generator (which generates a large number of different alternating magnetic fields), it is a potential advantage to use a solution which: (a) can work with a simpler EM transmitter (for example, one only transmitting 3-6 distinguishable fields), and (b) works without using many wires connecting between the sensor and the external processing unit.

In some embodiments of the present disclosure, a shape sensor is provided which uses as few as 2 wires connecting it to an external processing unit, while providing multiple inductance measurements which can be referred to conditions (magnetic field state) at specific regions along the sensor's curve. This may substitute for the alternative of making discrete individual inductance measurements along the curve.

Not only is the inductor constructed in a spatially inhomogeneous manner (that is, it has different coil winding pitch or other construction parameter which produces different baseline inductance per unit length at different positions along it), but also its high permeability non-linear magnetic core varies additionally according to the local magnetic field vector it is exposed to.

Upon suitable calibration and biasing, this results in a device that has certain expected overall inductance (e.g., as measured by probing circuit resonance) within reference EM field conditions which changes to different values depending on how much current is passed through the inductor. Adjusting the current changes the inductance, because a change in current changes the applied magnetic field in the inductor—which in turn acts on its own (non-linearly responding) core to change its properties. There is thus a calibrated current-inductance relationship which is alternatively expressed as a magnetic field-inductance relationship.

This relationship changes when the device is exposed to new external EM field conditions, since these also shift the permeability of the non-linear magnetic core. This can be probed with the same range of currents to which the device was originally calibrated, e.g., using a time varying (for example, sinusoidal or other waveform) current. For example, a 1 MHz sinusoidal current is provided. In some embodiments, current is provided according to another repeating waveform shape; for example, a triangle wave, a sawtooth wave, or another continuously or non-continuously varying signal waveform. Optionally, the driving circuit provides current directly as a targeted waveform. Optionally, the driving circuit drives a voltage to a selected waveform, with the associated current following according to overall circuit characteristics, potentially including distortion of the voltage waveform.

The results measured are indicative of a new relationship between current (or imposed magnetic field) and inductance, shifted according to the influences of magnetic field strengths in the sensed environment. More particularly, magnetic field-induced shifts in magnetic core permeability, whatever they are, may be assumed to account for shifts in the relationship.

Thus, the sensor overall comprises sensor regions, each providing to the sensor a variable electrical inductance, characterized by a baseline partial inductance (e.g., the inductance of the region in the absence of magnetization), a sensing partial inductance, corresponding to the strength of externally imposed magnetization, and an internal partial inductance, corresponding to self-magnetization when a current flows through the sensor region. The sensing and internal partial inductances may alternatively be referred to as variations to the baseline inductance (which may be negative or positive in sign). The variations may be referred to as “responsive to”, e.g., externally imposed magnetization and currents flowing in the sensor. To facilitate analysis making use of not-discrete sensor regions, references to inductance may be replaced by references to (linear) inductance density, with integration, e.g., along coil lengths, being treated separately.

Mathematically, a set of shifts that could account for the change can be determined, for example using non-linear optimization approaches. Calculations for this, in some embodiments, make use of knowledge of how inductance density is (inhomogeneously) distributed along the sensing region of the probe, and/or the inductance curve of the core material (e.g., its change in permeability as a function of magnetization). With suitably different inductances along the probe, there may only be one set of shifts which is physically plausible.

In some embodiments, this may be described informally as follows. Since each suitably distinct (although perhaps overlapping) “element” along the probe has a different inductance density, it also imposes a different bias upon itself when operated at a given current—and a different current-to-inductance curve. The curves may be similar in shape for different elements, but shifted from one another. In operation, shifts also include effects that local differences in external magnetic field vector (e.g., strength and/or direction) impose. As far as inductance changes go, each individual element acts like it is seeing a different current. When the curves are, in effect, added together by a measurement that treats them as one larger inductor, the features defined individually blend together. Since the shifts of the curves under calibration conditions place them in distinct baseline offsets (e.g., peaks in different places, linear regions in different places, and so on), then additional offsets induced from external magnetic fields can be assigned to specific elements.

These considerations show how magnetic field vectors (and/or their strength) may be localized along a 1-D parametric space defined by the longitudinal extent of the probe. To convert this to the curvature of that probe (and its parametric space) in 3-D space, the probe's detected magnetic field vectors are mapped to known spatial distributions of magnetic field vectors. The shape and position of the probe are given by offsets, rotations, and contortions which preserve consistency between magnetic field strength values measured by the probe, and those known to exist in 3-D space. This process may be assisted by knowledge of the probe's actual length, and assuming plausible limits on how the probe can move and bend. More than one magnetic field may be used, e.g., 3-6 magnetic fields operated at different frequencies, allowing the separation of field influences, e.g., using discrete Fourier transform methods, correlation methods, or another frequency decomposition method. The magnetic fields are preferably arranged in a region of interest so that different regions have combinations of field vectors which are at least in part decorrelated, e.g. the magnetic fields have magnitude gradients directed to have mutually orthogonal components. In this way, each region is magnetically “tagged” to be measurably distinct from its neighbors in any direction, and preferably to be measurably distinct from all other regions which a portion of the probe might enter. Variously directed magnetic field magnitude gradients may be interpreted as establishing respective coordinate axes that have known and/or predictable relationships with 3-D position coordinate axes (and optionally also with up to 3 rotational coordinate axes). In some embodiments, measurements of magnetic field strengths in combination are mapped to position and/or orientation coordinates through this relationship, also referred to herein as a “mapping” of field strengths and/or directions to spatial coordinates. The mapping relationship may be only partially known, e.g., estimated from theoretical operating parameters, and potentially only partially calibrated. Optionally the mapping relationship used is corrected and/or constrained by other factors such as distances known to be fixed, angles known independently, measurements at particular known spatial positions, and/or other data.

Although having particular value for shape sensing, the same physical principles are useful, even to detect single sensor positions. For example, in some embodiments, a sensor comprises a single sensing element, e.g., a single short coil wrapped around a high permeability non-linear flexible magnetic core, such as a high permeability magnetic wire (e.g., made of supermalloy). Through the measurement of variations in its inductance, the sensor provides readings of magnetic fields at a single position and orientation in space, for example, at the tip of an endoluminal steerable medical device, where the coil is positioned. The device may be a guidewire, in which case electromechanical properties of the flexible magnetic core material may themselves provide suitable steerability, torqueability, pushability etc. The same material's highly magnetic permeability is what makes the coil magneto-inductive, that is, enables magnetic field measurements at the coil through measurements of magnetic-inductance non-linear relationship. Exploiting the device's core material both for mechanical requirements of the steerable device as well as for its magnetic properties has potential advantages for the construction of ultra-thin devices which can be EM-tracked in 3-D. In effect, a portion of the body of the device needed anyway for its mechanical functions is used also as part of a magnetic sensor.

An aspect of some embodiments of the present disclosure relates to the integration of magnetic sensors into the body of long, thin medical instruments such as guidewires.

Endovascular guidewires are typically long and thin (for example, a typical guidewire can be 180 cm long and have an outer diameter of 0.36 mm), and depend for their function on mechanical function aspects such as:
Pushability: the ability to accurately transfer axial motion at the proximal end to the distal end.
Torqueability: the ability to accurately transfer rotational motion at the proximal end to the distal end.
Trackability: the ability to follow a tortuous path defined by the anatomy or another device such as a catheter.
An atraumatic design: avoid applying high pressure on the surrounding tissue as to not cause damage.
Kink-resistance: the ability to bend in tight radii without buckling.
Durability: the ability to bend and flex multiple times without failing.

While functions like torqueability and pushability require high torsional and axial rigidity, other functions such as trackability and atraumatic design require low rigidity and a smooth transition in rigidity between different sections of the guidewire.

In some embodiments of the present disclosure, a core and coil design is provided in which the core is integrated into the guidewire along a portion of its body (i.e., in place of at least a portion of other support materials). This has potential advantages for addressing functional tradeoffs, as well as the functions of kink resistance and durability.

The core may be provided as a solid straight wire providing mechanical strength and stiffness. The coil portion wrapped around it may promote flexibility, durability, and kink-resistance. Desired mechanical properties are achieved, in some embodiments, by balancing the different functional parameters promoted by the core and coil, for example using design parameters like material selection, core diameter, coil wire diameter, and/or coil average winding diameter.

In some embodiments of the present disclosure, a high permeability non-linear magnetic material chosen as the core material, for example permalloy, has roughly similar mechanical properties to stainless steel, a material commonly used for guidewire cores. Using an isolated conductive material, parameters of the coil can be tuned cooperatively with parameters for shape sensing by using the methods described above.

Any parameter affecting the inductance of the coil can be varied to create a single coil with varying properties that allow shape sensing with as little as two wires as described above, for example the core diameter, the average diameter of the winding of the coil, or the thickness of the coil wire can be changed along the length of the guidewire. Each of these parameters can be controlled individually to create variations in coil geometries. It is noted in particular that variable pitch winding of the coil may be useful to provide a smooth transition of flexibility, e.g., from relatively stiff to relatively flexible behavior, which may help to reduce stress focusing which potentially occurs at sharp transitions between relatively compliant and relatively stiff device portions.

Optionally, magnetic properties of the materials used for coil and core are varied to achieve the same effect. This parameter may be used in probe design whether the probe is used as part of a guidewire body or otherwise provided. For example the core can be made from a high permeability non-linear magnetic material mixed with in varying ratio of another material to create varying magnetic properties along the core's length. For example, supermalloy is an alloy composed of nickel (75%), iron (20%), and molybdenum (5%). A modification of a custom supermalloy flexible wire can be used as the magnetic core for the sensor, where the composition of the alloy varies along the sensor's length. For example, at the sensor's tip the composition can be as described above. Then, towards the proximal part of the sensor the alloy can be modified continuously or discretely (for example, by joining multiple segments of different compositions) such that the amount of nickel decreases in favor of the amount of iron or the molybdenum, for example, the composition changes continuously from 75% nickel, 20% iron, 5% molybdenum to 50% nickel, 45% iron, 5% molybdenum. Alternatively, the amount of nickel increases while reducing iron and/or molybdenum; for example, the composition changes continuously from 75% nickel, 20% iron, 5% molybdenum to 95% nickel, 0% iron, 5% molybdenum, or in any other suitable manner.

Optionally, an additional material is added (e.g., to the alloy) to tune mechanical properties of the core along the sensor's length as well as its magnetic permeability curve. Modification of the alloy affects the magnetic permeability properties of the material. This can support the solvability of an equation system (e.g., by further distinguishing the weighting of different regions of the sensor probe in total measured inductances) which is processed using data from total inductance measurements to produce separated measurements of a plurality of magnetic field values along the sensor's length.

Accordingly, varying the alloy composition along the sensor length in turn controls magnetic permeability properties of the alloy along the sensor's length. This may be additional or alternative to changing the winding pitch of the coil wire around the magnetic core, or modifying the core's diameter along the sensor's length. The changing composition helps shift the inductance curves of sensor portions away from each other, to promote sensing a plurality of magnetic field values along the sensor's curve.

When combined, more particularly, in a guidewire, a probe portion of the guidewire (which optionally comprises as much of the guidewire as is considered desirable, including the whole guidewire) can be constructed which both has the desired mechanical properties required for various clinical uses as a guidewire, and can be fully shape tracked in 3-D in real-time.

An aspect of some embodiments of the present disclosure relates to navigational aids to endoluminal interventions, and specifically to updating the deformation of a 3-D model of an anatomy-of-interest reconstructed from one or both of preoperative and/or intraoperative vessel imaging (such as angiogram, CT, etc.).

In some embodiments, data from a real-time shape-tracking method (for example, using a curve inductive sensor, or other sensor type) is used to track the deformation of the 3-D model in accordance with the real-time tracked shape of an endoluminal device (for example, a catheter, wire, guidewire, or probe) which is introduced into the anatomy of interest. This is then used to display the device on the deformed 3-D representation, which is continuously updated during navigation of the device.

For example of such application, during endovascular procedures a device such as guidewire, or microcatheter, or catheter, or other endovascular device, is introduced, for example through the femoral artery, into the vascular system; from which it is advanced through the luminous vessel structure to a target such as ABM, AVF, aneurysm, embolus, lymph node, or other desired target.

On the path from entry to target the device is navigated through bifurcations in the vessel and tortuous passages of ever-narrowing vessels, at times counter to the main direction of blood flow and while being deflected by pulsations of heart contractions, breathing or other causes.

Currently, this navigation is typically performed by first injecting contrast into the blood to visualize the vessels, which are otherwise translucent and usually hard to detect against bone and other opaque anatomical structures in standard fluoroscopy. Then, using single- or bi-planar fluoroscope, a set of static images of the now-temporarily-visible vessels is captured. Contrast does not last for long in the blood stream, so one or a combination of the static images of the contrasted vessel is saved and used as a “roadmap” which is overlaid on the live fluoroscopic image. This way the physician can simultaneously see both the radio-opaque device in the live stream, and the static overlaid image, to aid with navigation.

However, this is far from optimal, as the overlaid image is static and does not visualize elastic changes in the position of the vessel, as it is deformed by the device, as well as movement of the organ due to potential motion of the patient. This deformation may cause significant change in position of a bifurcation the physician is trying to navigate into. Further, the image is inherently 2-D, while the maneuver is in 3-D. In addition, device visualization is only achieved using fluoroscopy. Consequently, it is common for a physician to spend significant time trying to “trial-and-error” their way into a bifurcation. This in turn increases the risk of perforation of the vessel, prolonged procedure which may cause formation of emboli, increased exposure to radiation for patient and staff, and increased contrast dosage.

In relation to this disclosure, the system and method described may be used to generate real-time deforming 3-D visualization of the anatomy, in which the real-time location and orientation of the tip of the navigated device or the device's full or partial shape is displayed, to facilitate and aid navigation. Optionally, a projection of this 3-D visualization may be overlaid on live fluoroscopic image, or otherwise manipulated, to facilitate the same.

An aspect of some embodiments of the present invention relates to the use of flexible curve inductive sensing to track and display the location (position and orientation or full shape) of a device within an anatomy. In some embodiments, the tracking accounts for deformations of the anatomy. The terms “curve inductive sensor”, “flexible curve inductive sensor” both refer to “curve inductive sensor”.

In some embodiments, a tracking system comprises:
An endoluminal device fitted with one or a plurality of flexible curve inductive sensors (for example as described herein).
A processing unit, configured to perform algorithmic operations:
   reconstruct the 3-D shape of the endoluminal device;
   reconstruct a 3-D model of the vascular map (or other lumen map) using image inputs (e.g., fluoroscopic images, MRI images, CT images, ultrasound images, or another image type);
   register the 3-D tracked shape of the endoluminal device to the 3-D model; and/or
   infer shape changes to the luminal 3-D model based on the detected shape changes of the endoluminal device, using a deformation model.
A display, used to provide indications of current tracking status and/or provide navigational guidance.

In some embodiments, alternatively, a standard 2D roadmap is used, for example as routinely performed by using contrast and using one or two contrast images. The images are received and saved. Then, navigation is performed on these images (displays), without the need of dedicated 3-D map. In some embodiments, navigation is performed on the 2-D roadmap. In some embodiments, this is allowed because the system utilizes a 3-D re-projected tracked device (see EM to X-ray registration below for more details). In some embodiments, a potential advantage is that it potentially reduces (optionally completely eliminates) the need for the continuous use of repetitive X-ray imaging to track the device shape during navigation between contrasts. It may eliminate radiation by 90% and it keeps the current operator workflow.

Additionally or alternatively, the tracking system uses another type of position and shape sensing of the endoluminal device, for example: EM tip sensing, multi-sensor EM shape sensing, fiber optic shape sensing, passive RF, detectable magnets, ultrasound-detectable markers, fluoroscopic shape tracking, or another localization method.

In some embodiments, a 3-D model of a luminal structure is generated from images visualizing the lumen in some baseline state. The images may be, for example, fluoroscopic, CT, MRI, ultrasound, or images generated using another imaging modality.

For example, in the case of reconstruction from fluoroscopic angiogram images, the image input comprises 2-D X-ray projections of blood vessels into which fluoro-opaque contrast material was injected, imaged for a plurality of projection angles. The 3-D luminal structure can be reconstructed from the 2-D projections, for example by using iterative back-projection methods, cone-beam reconstruction methods, inverse Radon transform or any other suitable method.

In some embodiments, each of the 2-D projection images is known to have been obtained from a specific angle of the fluoroscope relative to the imaged region, and/or from a specific position and orientation of the fluoroscope in space, relative to some reference coordinate system.

Using this positional information and the 2-D projection images themselves, a 3-D luminal structure model is constructed which accounts for the arrangement of structures shown in the images. For example, if the 3-D model were to be re-projected into 2-D images, in a similar manner as the fluoroscopic projections were physically generated (that is, by tracing virtual X-rays in space based on the fluoroscope's camera calibration, for example, according to Beer-Lambert law), the 2-D re-projected images would match those that were acquired.

The problem of finding such a 3-D model can be solved using optimization methods, for example, by iterative back-projection methods. Once a 3-D volumetric image is found that sufficiently matches the observed projections, the vessels of the model can be segmented in the 3-D volume. From this, their respective volumes in 3-D relative to a reference coordinate system can be identified. The 3-D segmented vessels can then be processed to a representational form; for example, their centerlines and radii are extracted to be used by other algorithms, such as skeletal-based deformation tracking of the vessels.

In another example of processing, a 3-D skeleton model of the vessels is found directly which conforms with the observed 2-D fluoroscopic projections. In this embodiment, a 3-D skeleton model is searched directly, such that its 2-D projections (using the same fluoroscopic camera locations) would be consistent with the observed 2-D projections. Volumetric properties such as vascular radius, if needed, may be added to the skeleton, for example, by mapping skeleton segments to vascular regions shown on the 2-D projections.

When the image source is itself 3-D (e.g., as for 3-D MRI and CT modalities), segmentation algorithms typical of the modality and luminal structure type may be applied as necessary to produce a skeletonized representation of the luminal structure.

During a procedure using an endoluminal device, the endoluminal device's 3-D shape, or tip, or another set of locations linked to the device are tracked in 3-D. Tracking may use a flexible curve inductive sensor, flexible curve resistive sensor, multiple EM sensors, fiber optics shape tracking, fluoroscopic segmentation techniques, or another suitable method.

In some embodiments, the device's 3-D shape is displayed as if appearing inside a visualization of the 3-D luminal structure model, optionally along with some or all of the image data used to generated the 3-D luminal structure model, and can be viewed from and position and orientation of a virtual 3-D camera.

This may involve further operations to match coordinate systems. For example, the 3-D luminal structure model may be generated from a preoperative scan. The device's reconstructed shape is then not inherently linked to a coordinate system in common with the 3-D luminal structure model. In this case the device can be registered to the luminal structure model, for example using cloud-based supervised or unsupervised registration methods (e.g., methods based on total error minimization). Conversely, once registration has been performed, the preoperative 3-D luminal structure model can be transformed to the same reference coordinate system used for tracking the device, after which the device position can be displayed together with it.

Coordinate systems do not necessarily need to be explicitly matched in some embodiments of the present disclosure. For example, if both the device's 3-D shape and the 3-D lumen structure model are reconstructed from 2-D fluoroscopic projections, then they may both reside initially in the same reference coordinate system. In an intermediate case, the device's 3-D shape is separately known, but also in part visible by its projection onto the 2-D fluoroscopic images. The 2-D projections of the device may then be used as partial information to help register the separately known 3-D shape of the device's 3-D shape to the 3-D lumen structure model.

In some embodiments of the present disclosure, the system is configured to account for deviations of the 3-D luminal structure map from its source image which may occur over time, e.g., due to normal physiological movements such as breathing, heartbeat, and/or peristalsis; due to posture differences (e.g., different reclining positions); due to passive shifts in internal organ position; and/or due to movements caused by one or more medical procedures themselves.

In some embodiments, the real-time tracked 3-D shape of the endoluminal device is used as an input to help determine deformations of the 3-D luminal structure model. This determination, along with visualization feedback, may also occur in real-time. Real-time may mean, for example, updated at least every second, at least 5 times a second, at least 10 times a second, at least 30 times a second, or more often. Updates may lag inputs by about the period of an update, or a larger or smaller amount.

In some embodiments, a skeletal-based deformation model (or another suitable organ deformation model) is evaluated, applying the device's 3-D shape as a constraint on the deformation model. The deformation model is derived from and/or applies to the luminal structure model, adding in information governing how the luminal structure model deforms—for example, it may specify degrees of freedom corresponding to elasticity, angular deflections at junctions, and/or flexibility of segments. Constraint of the actual deformation to match the 3-D shape of the device may comprise an assumption that the device always resides inside a lumen represented by the model.

Accordingly, a deformation of the luminal structural model is searched, consistent with the deformation model, such that the device's shape fits along some portion of the deformed lumens. Additional constraints specified in the deformation model itself are balanced to otherwise preserve an equilibrium anatomical structure (e.g., bifurcation angles, lumen length etc.). From any given current state, the search problem to find a suitable deformation can be solved by error minimization techniques.

The equilibrium anatomical state may be the original state of the anatomical structure model, or another state, e.g., adjusted from the original anatomical structure model's state according to new information from images, patient monitors, or another source.

In some embodiments more particularly, the equilibrium anatomical structure against which deformation parameters are calculated is remodeled as appropriate to account for updated observations of the shape of device(s) being monitored. For example, if a luminal region is deformed from the equilibrium luminal structure model state in a manner which is not accounted for by mechanical properties of the probe and/or forces exerted upon it, this may be accepted as indicative of a new equilibrium state (e.g., a change which persists regardless of the probe's presence). Being “accounted for” may be assessed by a rough rule of thumb; for example, initial entry into a modeled region that discovers it to have a new configuration (e.g., a new branch angle at a bifurcation) may be taken as evidence of a shift in equilibrium structure, as distinguished from a deformation which the probe itself imposes.

Additionally or alternatively, in some embodiments, there is a mechanical model associated with the probe. This may describe, for example, the probe's response to: pressures from behind or ahead, twisting about its longitudinal axis, and/or its tendency or resistance to straightening and/or curving. Dynamic forces on the probe (e.g., as used to navigate it) may in turn be measured, for example using strain sensors in locations near where the device is manipulated. Alternatively or additionally, they may be modeled within an envelope of plausible forces which could be acting on the probe during any given observation or sequence of observations of its state. Observed deformations inconsistent with the probe's own mechanical model (or at least in part unlikely, in view of it) may be in part attributed to a change in equilibrium state of the luminal structure model, and/or of the deformation model associated with it.

Display updates do not necessarily await new measurement data, or new full recalculation of deformations input data (“key frames”). For example, during intervals between full recalculation of deformations from new input data (“key frames”), visually presented updates may be provided by extrapolating from a recent trend in movements, assuming it will continue. For example, the first, second, and/or further derivative of movements may be extrapolated to estimate a next position. Damping or other modification of recently observed trends in the data may be applied to reduce overshoot. For example, a machine learning product may be trained on previously recorded tracking data, and used to adjust tracking displays between key frames to more closely approach the next expected fully-calculated state. Additionally or alternatively, previous measurements of deformations associated with cyclical physiological movements are incorporated into extrapolated movement displays. In some embodiments, a deformation state extrapolated ahead of the incorporation of actually measured data is used (whether or not it is also used in display updates) as an initial state for an error minimizing algorithm once the actually measured data becomes available (e.g., used instead of beginning from the last key frame state of the model, or instead of beginning from a presumed equilibrium state of the model). This may assist in speeding convergence, which, in a virtuous cycle, potentially in turn allows a faster rate of key frame calculation.

Accordingly, in summary of the foregoing: some embodiments of the present disclosure provide solutions that allow sensing, tracking and displaying the real-time in vivo deformation of endoluminal structures by using previously acquired images of the structure, and applying the real-time 3-D reconstructed curve of a device present onto the imaging, for example for the purpose of navigating said device endoluminally.

Terms used herein to refer to properties of magnetic fields may be understood according to the following explanations and definitions.

Local magnetic field strength in its totality may alternatively be referred to as the magnetic field vector's magnitude, and direction may be referred to as that magnetic field vector's orientation. However, references to magnetic and/or magnetization field strength as such include the potential for signed values (negative or positive). This allows, for example, magnetic fields from a plurality of sources to potential adding up in opposite directions.

Where measurements of a magnetic field vector are potentially ambiguous as to a particular magnitude and orientation (e.g., because the sensor is anisotropically sensitive, measuring the vector anisotropically so as to indicate only a portion of its full magnitude), reference to the field's “strength” may be considered as a shorthand for what is measured, though this may in some embodiments be corrected to the entire local magnetic field vector by applying knowledge of the sensor's anisotropies and other available constraints. Reference to measurements of magnetic field, whether referred to as of strength, magnitude, vector, direction, or orientation, should be understood to refer to measurements of local magnetic field, e.g., its properties in a particular region of space to which the measurement is assigned. This may be, for example, a region including the position of the measurement device, or the position of a relevantly affected portion thereof. Inhomogeneities of magnetic field vector existing within that region are generally subsumed within a single measurement (e.g., averaged, according to whatever weighting the construction and sensitivity of the measurement device imposes). Reference to the strength or magnitude of a time-oscillating magnetic field vector may be understood as referring to its root mean square (RMS) strength/magnitude, also sometimes referred to as its DC strength/magnitude (or “value”, in whatever terms are set by the conditions of the measurement). Magnetic field amplitude refers to half peak-to-peak amplitude of a time-oscillating magnetic field vector (e.g., if non-rotating and centered on a magnitude of 0, half the sum of the peak magnitudes in either direction).

Furthermore, in relation to cases wherein a magnetic field is, more particularly, generating effects on the permeability of a material, it may be alternatively referred to herein for emphasis of this point as a “magnetization field”, associated in turn with properties such as “magnetization field strength”. However, uses of terminology based on “magnetic” should not be construed as thereby excluding magnetization effects. Also, the term “magnetization” by itself may be used to refer to magnetization field strength.

Permeability has units of inductance per unit distance (e.g., H/m in SI units). Along a certain axis, such as the longitudinal axis of an elongated sensor body, permeability may also be referred to as a measure of “inductance density”, or “linear inductance density”. Inductance density as such, however, may be the product of further parameters, such as coil geometry and current. Due to the close relationship between inductance and permeability changes, “inductance” may be referred to as changing when permeability changes, and vice versa.

Where the term “average” is used in reference to a measurement of time-oscillating magnetic field, root mean square averaging is assumed unless otherwise specified. In a strict sense, average magnetic field direction or orientation of a time-oscillating magnetic field may be null. Where this is a potential concern, reference to direction/orientation may be understood to refer to the orientation of an axis of oscillation of the instantaneous magnetic field magnitude. Unless otherwise specified, a reference to a time-varying magnetic field may be understood as emphasizing the case of a time-oscillating magnetic field (e.g., as a preferred embodiment).

An aspect of some embodiments of the present disclosure relates to spectrally multiplexed, spatially resolved sensing of environmentally induced local changes in the resistivity of a sensing material. In some embodiments, the sensing material is provided as part of a medical device probe, for example, a guide-wire, catheter-delivered tool, or catheter portion.

In some embodiments, an environmental parameter that is sensed by causing the resistivity changes is local magnetic field strength and/or direction. Additionally or alternatively, in some embodiments, another environmental parameter induces resistivity changes; for example, temperature. In some embodiments, the environment interacts with the sensing material to generate stress and/or strain in the sensing material, and the resistivity changes in response.

Certain materials are particularly susceptible to changes in resistivity in response to particular conditions, and/or commonly used as sensing material in applications that use these changes in resistivity. For example, permalloy (e.g., an alloy comprising about 80% iron and 20% nickel) is noted for having up to about a 5% variation in electrical resistance in response to an applied magnetic field. Furthermore, its magnetoresistance properties are anisotropic. This makes it useful for detecting both magnitude and direction of the local (intersecting) magnetic field. Thermistors (devices engineered for their relatively large and/or predictable resistivity changes as a function of temperature) are commonly produced using powdered metal oxides and/or silicon as their sensing material. The temperature coefficient of resistivity for material used in a thermistor may be, for example, about 5%. Piezoresistors display a change in resistivity as a function of strain; various semiconductor materials are commonly used as the sensing material in their commercial manufacture.

Sensing materials are available in different forms depending on their other material properties and available manufacturing technologies, for example continuously in the form of strips, tapes, wires, and/or sheets; and/or discretely, for example, in the form of sensor packages. Herein, the term “variably resistive sensing material” (or “sensing material”) refers to a material particularly selected in technological practice for its variable resistivity properties, i.e., its relatively large (in magnitude) coefficient of resistivity with respect to a change in a property such as local magnetic field, temperature, and/or strain.

In some embodiments of the present disclosure, a flexible resistive sensor is provided which measures a physical quantity (e.g., temperature, strain, magnetic field) along the full longitudinal extent (curve) of the sensor, optionally without using a plurality of discrete sensors along that curve. For example, in place of a plurality of separate films along the full of the sensor, the sensor comprises a continuous long film (for example, 1 mm wide and 30 cm long) or a set of discrete films connected in series; for example embedded on a flexible printed circuit (FPC) which gives the sensor shape flexibility. The sensor can be embedded in a device, for example, wrapped around a catheter. Suitable readout and processing of data measured from the sensor potentially provides real-time shape tracking of the catheter or other device so-equipped.

In the pursuit of miniaturization of sensing devices—in addition to the problems of miniaturization itself—there are potential drawbacks and/or difficulties arising in device aspects such as cost, manufacturability and/or reliability; particularly when a plurality of sensing locations are to be provided. There is also a problem of how these sensing locations may be addressed. In any of these cases, it is a potential advantage to reduce componentry, and/or to reduce numbers of individual connections.

In some embodiments of the present disclosure, a form of spectral multiplexing is used which allows a series-connected array of variably resistive sensing material portions to share electrical interfacing connections to a supporting measurement system in common. A potential advantage of such a structure is a reduced need for connections leading directly into the body of the series-connected array. Connections made from the ends, for example, can be used, in effect, to address sensing regions centered upon different locations all along the series-connected array.

In brief: in some embodiments, the impedance of the series connected array is measured at DC, and/or at one or more AC frequencies, for example, using a Wheatstone bridge. In some embodiments, using different measurement frequencies has the effect of integrating the resistivities of the sensing material portions with different respective weights (contributions) in terms of their effect on the signal response measurement. Additionally or alternatively, in some embodiments, the resistances of the series connected array are differentially sensitive to oscillating signals in the environment (e.g., because different resistive areas are exposed to different magnetic field oscillations, and so change their resistance with different timings).

In some embodiments, this is in part analogous to what happens when measuring the frequency dependence of the impedance of a transmission line. In the extreme of a DC (constant) driving signal, for example, all passive resistances are theoretically expected to contribute additively to the total resistance signal. As driving frequencies increase, however, circuit reactances (which, in the case of a simple transmission line, are understood to be present intrinsically) tend to shift this proportionality, e.g., to favor increased contributions from the near end(s) of the series-connected array.

Distinguishing, however, for some embodiments of the present disclosure, is that the “transmission line”—the series-connected array of variably resistive sensing material portions—is intentionally non-uniform. Moreover, as it is used for sensing, it is variably non-uniform: different portions change their resistivity differently depending on the local conditions that affect them.

There is, in short, information which, under suitable conditions, may be extracted to help characterize the present state of resistivities of different sensing material portions. Of course, the reactances themselves contribute to the frequency dependence of the measured signal, “contaminating” the information. However, with the exercise of suitable constraints and prior knowledge about the probe which comprises the sensing material portions, a computational process can potentially determine from this information a physically plausible model of the pattern of resistivity along the sensing material portions.

In some embodiments of the present disclosure, the sensing material portions are optionally discrete (e.g., individually packaged components) or continuous (e.g., in tape or strip form).

The concept of a “sensing region” is a model-dependent element distinct from the particular physical structure (e.g., discrete or continuous) of the sensing material. In some embodiments, there are identified within the series-connected array of sensing material portions a plurality of distinguishable sensing regions. The resistivity associated with a sensing region optionally is a single (e.g., weighted average) value, and the value is optionally associated with a particular (e.g., “center”) location along the array of sensing material portions. However, the collection of portions of sensing material contributing to sensing regions optionally cross physical boundaries that may exist between portions (e.g., they may include more than one discrete sensor). These collections optionally overlap with one another, wholly or in part. What distinguishes the locations of different sensing regions may be characterized as differences in the weighting which applies to contributions of the sensing material portions.

In some embodiments, and also in distinction to a normal transmission line, a series-connected array of variably resistive sensing material portions is embedded in a sensing circuit which is configured to enhance spectral impedance differences.

In a basic example, reactance may be controlled along the probe by adding small components (e.g., capacitors) at spaced intervals. The spaced intervals may be constant or non-uniform. Their capacitance values and/or the spaced intervals at which they are placed may be selected to differ from one another in relatively large steps. The capacitance values and/or their spaced intervals may be selected, for example, so that different portions of the probe (including different sensing material portions) are recruited into resonance at distinguishably different frequencies. These frequencies may be used in turn to determine which driving frequencies are used to operate the device for measurement.

In some embodiments, electrical properties of the sensing circuit are manipulated in another way which potentially increases the distinctiveness of different sensing regions for the de-multiplexing procedure. For example, insulating wire coating thicknesses may be varied, and/or conductor thickness itself may be varied. Optionally, arrangements of the sensing material itself may be changed. For example, the sensing material may be straight or coiled (optionally differently in different longitudinal locations along the probe), and if coiled, may be coiled at different pitches in different locations along the probe. “Coiled” includes one or both of helical coiling of a length of the sensing material by itself around and along an axis (e.g., as in a coil spring), and twisting of a length of the sensing material together with another conductor—for example, a return wire for the sensing circuit, and/or another length of sensing material. Twisting in particular may be used to create a twisted pair configuration of the sensing circuit, and the degree of twisting (e.g., how many twists per cm are provided) may itself manipulate inductance and/or capacitance, optionally without the addition of discrete inductor and/or capacitor components.

It should be noted, furthermore, that the arrangement of the sensing material may affect its sensitivity, and/or what types of forces it senses. For example, a coil of a strain-sensitive sensing material placed near the tip of a probe is potentially well suited to detect axially compressive forces, while a straight length of such material may be better suited to detecting bending forces. Anisotropic magnetoresistive material can be oriented to detect magnetic fields better from one direction than another. Where the magnetoresistive material is wound around a longitudinal axis of the probe (e.g., a wound strip), the directional sensitivity may vary as a function of distance along the probe. Winding such sensing material at different pitches at different longitudinal regions along the probe may also assist in enhancing regional distinguishability.

It should be understood that there are aspects of probe calibration which potentially affect the process of de-multiplexing (e.g., by providing constraints and/or baseline values for the device). In some embodiments, the probe and/or sensing circuit comprise additional elements which assist in probe calibration. For example, where resistivity of a first sensing material is sensitive primarily to magnetic fields, but secondarily to strain, a second sensing material may be provided which is sensitive primarily to strain, and secondarily (if, practically, at all) to magnetic fields. Responses of the second sensing material may, for example, be arranged to counteract those of the first sensing material (e.g., with an opposite sign), or the second sensing material may be provided in a second sensing circuit, and measurements from the second sensing circuit used to constrain the solution which yields resistivities along the sensing material of the first sensing circuit. Furthermore, wherever two different lengths of sensing material can be compared (e.g., because they are immediately adjacent longitudinally, or running longitudinally alongside each other), differences between them may be indicative of local conditions. For example, strain-sensitive sensing material on an inner radius of a bending probe may experience compression, while strain-sensitive sensing material on the outer radius may experience strain.

An aspect of some embodiments of the present disclosure relates to the use of spectrally multiplexed, spatially resolved readout of resistance changes in a magnetoresistive material for position finding.

In some embodiments of the present disclosure, not only is a physical property measured along a probe; but also, the probe extends along an at least partially unknown curve in space. Determination of that curve is constrained using the measurements of the physical property. In some embodiments, moreover, the physical quantity is itself specially arranged to increase the amount of positioning information available from the measurements.

In some embodiments, accordingly, an environmental parameter is manipulated in order to provide a spatial frame of reference that allows measurements associated to particular longitudinal positions along a sensor (e.g., a sensor constructed as just described) to also be associated with particular regions in space. In some embodiments, the manipulation comprises inducing EM (e.g., magnetic) fields throughout a region being navigated by a probe such that there are expected and generally distinguishable field properties measurable at each location within the region. This may involve inducing a plurality of EM fields, from different directions, and with different frequencies, phases, and/or duty cycles. The measurements from the probe may thus be multiplexed not only according to the readout signals generated, but also according to properties of the environment such as the local intensity of each of a plurality of magnetic fields distinguished also by their frequency and/or direction. In this approach, resistances are determinable as just described. The resistances in turn vary characteristically according to where in the complex superimposition of imposed magnetic fields they are. This information allows the position and shape of the sensor to be determined.

However, in some embodiments, spectral de-multiplexing of the probe impedance into an “intermediate” resistance mapping is not performed.

The total DC resistance of the sensing material amounts to the line integral of the resistance density (i.e., the resistance per unit length) along the sensor's longitudinal extent. This provides a single measurement of resistance along the sensor's whole longitudinal extent. In a simplified case, where only one part of the probe is affected by the imposed fields, knowing that this part interacts with a certain combination of electromagnetic field vectors provides the information that this part of it must be at a certain location “tagged” with that combination, providing position information in its own right. When effects on several parts are combined into one reading, however, the problem is made more complicated by the superimposition of effects, so that the problem becomes one of determining a position which would be consistent with the aggregate measured effect of the imposed fields. This does not necessarily have to proceed through a stage of isolating resistance changes as such in particular regions of the sensor.

For use in electromagnetic (EM) shape tracking, an EM fields generator which transmits a large number of different (that is, spatially distinct in shape) AC fields in different frequencies, (for example, 30 fields) is provided. A different total DC resistance (or optionally AC impedance) will be measured at each of the frequencies used. These total resistances can be distinguished by temporal frequency decomposition, for example, using DFT (Discrete Fourier Transform) methods, correlation methods, or any other suitable method. These values are indicative of the sensor's full shape in space, relative to the transmitter. Under certain smoothness assumptions of the shape sensor, the sensor's full shape can be solved to a good accuracy based on the various (for example, 30) sensed values.

For example, with just three orthogonal fields (and amplitudes, computed by analyzing temporal frequency decomposition of total resistances converted into magnetic fields) to work from, it would potentially be possible to assign an average center location to the probe, but not its full shape or orientation. With resistances to fourth and further fields added, however, only certain shapes and/or orientations of the probe will be consistent both with that average center, and with the additional measurements. The more fields used, the more constrained is the shape and position of the sensor. However, it may be understood that generating a sufficiently large number of AC fields is potentially cumbersome for a variety of reasons (e.g., space and setup considerations), so that it is a potential advantage to provide a solution which works with a smaller number of EM fields (e.g., three fields).

In some embodiments, the environmental parameter is more focally manipulated. For example, one or more energetic foci (optionally one or more moving or scanning foci) may be generated in the vicinity of the probe, and used to identify positions of any selected portion of the probe based on when, where, and to what extent resistive changes along the probe occur, relative to where the one or more energetic foci are created. The energetic foci may, for example, comprise regions of heating, generated, for example, using focused ultrasound, focused EM radiation, or another energy source.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Spectrally Multiplexed Longitudinally Extended Sensor

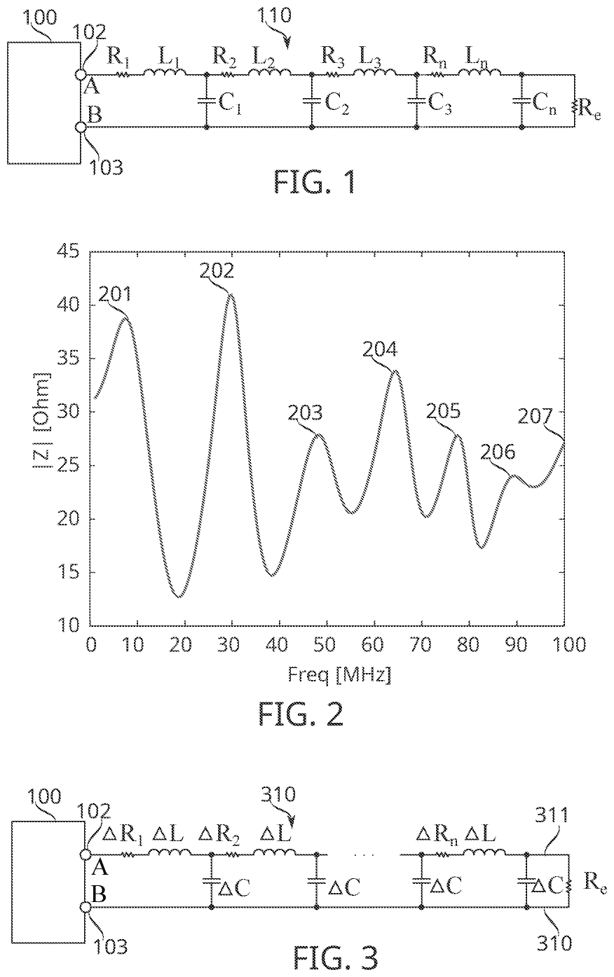

Reference is now made to FIG. 1, which schematically illustrates 2-lead sensor 110, with electrical properties divided to discrete units; either actually, or for purposes of description and analysis. The variable resistance of each sensing material portion is indicated by each resistance Rᵢ up to Rₙ. Reactive properties (Lᵢ, Cᵢ up to Lₙ and Cₙ) are configured to yield different total impedances under different test frequencies, in an overall configuration optionally similar to the electrical circuit topology of a transmission line. Sensor 110 is shown terminated with a termination Rₑ (a resistor, or another arrangement of terminating components). In some embodiments, sensor 110 comprises a flexible printed circuit (FPC).

In some embodiments of the present disclosure, sensor 110 comprises a long resistive film (or other form of sensing material), for example embedded as a layer of an FPC. At least for purposes of understanding and description, this sensing material can be considered as broken into discrete portions connected in series, each with its own resistance Rt. This may correspond to an actual physical division, although the sensing material is optionally continuous. The sensing material can be connected to an external processing unit and readout controller 100) using, e.g., two connection terminals 102, 103 (also labeled A, B).

Reactances of the circuit (due to capacitances Cᵢ and inductances Lᵢ, optionally of different values), may be (in whole or in part) intrinsic to the arrangement and physical properties of the resistive film and/or wires interconnecting portions thereof. Physically, reactances and/or resistances may be distributed continuously, but this can be represented for analysis to a selected accuracy by dividing the continuous distribution into any number of sensing units required (continuously distributed properties are discussed further in relation to FIG. 3). Optionally, the reactances are at least in part provided by added components; for example 0201 (0603 metric) SMD (Surface Mount Device) components.

The resistances Rᵢ (resistance of the i^{th} circuit unit, including resistance of a portion of the sensing material) are unknown, at least in part. In particular, at least the changes from some baseline due to environmental influences one the resistance of the sensing material are unknown. Determining these changes is of specific interest in some embodiments of the present disclosure, in order to extract localized sensing information about the environment. In order to produce multiple and differentially informative impedance measurements of the sensing material which may allow Rᵢ to be inferred, a multispectral approach is used. The processing unit 100 measures the complex impedance (resistance and reactance) of the sensor 110 at multiple frequencies; for example, from 1 MHz to 100 MHz. The measurements can be obtained, for example using an AC Wheatstone bridge. Optionally, measurements are repeated, for example, at a rate of about 1 kHz, or a higher or lower frequency.

Due to the reactances being physically associated to different locations relative to the sensing material portions of the circuit units, the unknown variable resistances of sensor 110 are combined differently (e.g., with different weights) for each test frequency. This produces a system of non-linear equations on the unknown Rᵢ elements, with known coefficients determined by the known values of the reactances and the test frequency. The system of equations can be solved, for example, using least squares optimization methods, permitting the values of the Rᵢ elements to be recovered.

More formally: denote by Rᵢ the unknown variable resistance of each i^{th} resistive film unit or other resistive sensing material portion of sensor 110. The resistive sensing material may be, for example, magnetoresistive, in which case its resistance in some location is indicative of the magnetic field at that location. Optionally or alternatively, the sensing material is thermo-resistive or of any other variably resistive nature. Lᵢ and Cᵢ are the known (and optionally assumed constant) inductance and capacitance values respectively distributed along the sensor; for example, arranged as depicted in FIG. 1.

Even if not actually constant, they may still optionally be treated as such, with an effects of their variation being collected, e.g., into the estimated varying values of Rᵢ. Assuming that such variation (if any) is at least correlated with variation in Rᵢ, error this may create can be corrected by suitable calibration. For example, an externally applied magnetic field may affect the inductance of some magnetoinductive films or nano coils. In any such case, demultiplexing methods described herein are adaptable as necessary to measuring the general varying complex impedance of films connected in series along a sensor. The methods are by no means restricted to measuring just the real-valued resistance of a plurality of discrete films or a continuous film as described above. In general, Rᵢ and R(x) can be thought of as complex numbers representing the varying complex impedance of a film and they can be measured using the exact same methods as were described.

Optionally, the circuit contains a termination resistor Rₑ, chosen, for example, according to the classical formula

$${R}_{e} = {Z}_{0} = \sqrt{\frac{L}{C}}$$

to match a (resistance-neglecting) characteristic impedance of the sensor, considered as a transmission line. This can help prevent measurement confusion due to signal reflections from the distal end of sensor 110. Where resistances introduce significant frequency dependence of the characteristic impedance, they are optionally also taken into account. Optionally, termination comprises additional passive components to better match terminal impedance through a wider range of the spectrum of driving frequencies used.

Sensor 110, in some embodiments, comprises an FPC with two lead terminals 102, 103. The lead terminals connect to an external processing unit 100. If the circuit were without reactances, then the measured resistance would be the sum of all Rᵢ's plus Rₑ, regardless of the impedance test frequency. This would preclude spectral de-multiplexing. However, since there is reactance, the total complex impedance between terminals 102, 103, denoted by Zₜₒₜ is frequency dependent. Zₜₒₜ can therefore be written as a function:

$${Z}_{tot} = {Z}_{tot} ( ω , {R}_{1} , {R}_{2} , … , {R}_{N} )$$

Where ω is 2πf and f is the test frequency. For any choice of ω and resistor values Rᵢ the total impedance Zₜₒₜ can be computed by electrical simulation (for example, utilizing Ohm's law and Kirchhoff's circuit law). Insofar as the values of the reactive components inside the sensor are known, the total impedance can be estimated for any choice of values. The external processing unit measures Zₜₒₜ at multiple frequencies, for example from 1 MHz to 100 MHz, resulting in M (M in a range of 10-100, for example) measurements Zⱼ at frequencies ωⱼ. For each measurement j between 1 and M, the measurements satisfy:

$${Z}_{j} = {Z}_{tot} ( {ω}_{j} , {R}_{1} , {R}_{2} , … , {R}_{N} )$$

Where ωⱼ is the known test frequency of the j^{th} measurement and Rᵢ are the unknown variable resistances of the i^{th} resistive element (e.g., film unit). Each of the M measurements contributes an equation (usually non-linear) on N unknown variables Rᵢ, for a total of M equations on N variables. If M≥N and if the system of equations is regular enough (which depends on the choices of Lᵢ, Cᵢ) then the system is solvable using non-linear methods and the Rᵢ solution is unique.

### Resolving Different Longitudinal Positions

Reference is now made to FIG. 2, which shows an example of a total impedance vs. frequency relation of a sensor with eight resistive elements and reactive components, according to some embodiments of the present disclosure. Optionally, the resistive elements comprise a single continuous film of sensing material, or serially-interconnected segments of sensing material (e.g., film units). The reactive components are provided as discrete components in an arrangement embodying the circuit structure of FIG. 1. In this example, the capacitors used are 150 pF and the inductors used are 70 nH. A termination resistor is chosen with Rₑ=21.6Ω. These values are chosen in advance. The total impedance vs. frequency profile shown is for eight film resistance values Rᵢ: [3,1,4,1,5,9,2,6]Ω (while these happen to be initial digits of pi, they are selected merely as examples). The whole device is optionally constructed as an FPC.

Frequency response of the circuit is tested for a plurality of frequencies in the range of 1 MHz to 100 MHz. The frequency dependence of impedance is shown continuously, e.g., interpolated between measurements. With appropriately selected values, simulations predict that a plurality of distinguishable peaks arise in the graph of the sensor's frequency-dependent impedance. Different resistive elements (and changes in their resistance) contribute differentially to determining the magnitude of each peak.

Peak impedance values are not, in general, solely determined by the closest resistance value, but insofar as the weighting of resistance contributions changes from frequency to frequency, information about what pattern of resistances is consistent with the observed measurements is embedded in their values. It should understood that it is not necessary to confine analysis to the use of peaks, but these are notable for their visual correlation in amplitude with corresponding resistances.

Adding inductances as discrete components at discrete locations potentially helps emphasize the distinct contributes of resistive regions between them. However, this is not an absolute necessity, depending on noise conditions, the relative size of resistive changes, and other factors influencing signal to noise.

In order to further assist the solver, additional constraints can be added. Optionally, these relate to plausible constraints on the behavior of the physical quantity being measured. For example, with a longitudinally extended temperature sensor, the temperature can be assumed to be smooth along the curve to a certain extent (e.g., not varying more than a certain amount within a certain distance, and/or having a gradient not varying more than a certain amount within a certain distance). Time-dependent behavior is also an optional constraint; e.g., various forms of the assumption that moment-to-moment differences remain within some restrictive bounds.

Since the values of Rᵢ are solved using non-linear optimization methods, additional constraints may be introduced into the non-linear solver, in various ways; for example, by adding a weighted smoothness error term |Rᵢ−Rᵢ₊₁| in the total energy function being minimized.

A similar approach may be used with magnetoresistive sensing, exploiting contiguity and/or limits on rates of change of the magnetic field along the sensor's longitudinal extent.

In some embodiments, the total impedance of the circuit is measured for each frequency separately (e.g., at different times, driving the circuit with a different single frequency). Optionally, the impedance of a plurality of frequencies (e.g., all tested frequencies or any portion thereof) is measured in a single test. For example, a 1 MHz square voltage signal can be fed into the circuit. A 1 MHz square voltage signal contains a superposition of a 1 MHz sine/cosine wave and its harmonics, for example between 1 MHz and 100 MHz. By analyzing the circuit's 1 MHz periodic current function, the total impedance of the circuit for each frequency within the frequency range can be calculated. Optionally, another superposition of a plurality of frequencies is used.

In some embodiments, in contrast to direct embedding a sensing material in a single strip (e.g., a resistive film) along the longitudinal extent of an FPC, the sensing material is embedded inside discrete elements, for example, as a 0201 SMD component, similar to other standard passive electrical components. The 0201 SMD resistive component is special in the sense that it its resistance is largely due to a material selected because its resistance undergoes relatively large and predictable changes under certain physical conditions. For example, a magnetoresistive element is selected which changes its resistance by up to several percent (e.g., 3%, 4%, 5%, or more), depending on the external magnetic field inside which it is located. Placing the resistive film inside a small discrete element is potentially advantageous since it concentrates the resistive film at a certain focal location along a sensor (due to its small size).

### Continuous Spectrally Multiplexed Longitudinally Extended Sensor

Reference is now made to FIG. 3, which schematically illustrates 2-wire sensor 310, with electrical properties divided to discrete (but infinitesimal) units for purposes of description and analysis. Variable differential resistances of the sensing material (provided, e.g., as a continuous film unit) to be measured are indicated by ΔRᵢ. Virtual differential reactive elements are indicated by ΔC and ΔL. The sensor is shown terminated with a termination resistor Rₑ.

The use of the δ prefixes indicates an emphasis in FIG. 3 on the continuously sub-dividable arrangement of electrical properties in some embodiments of the present disclosure. In relation, for example, to FIG. 1, it is described that the physical construction of the sensing circuit need not physically package resistances and reactances into discrete units. This construction may be used in all or some portions of the sensor. Accordingly, spectrally multiplexed operation of the sensing circuit may rely on inherent reactance. Potentially, this allows the sensor to be produced with a reduced maximum diameter, and/or reduced diameter in one or more particularly size-sensitive areas, for example, a reduced diameter throughout a distal tip region of the sensor.

In some embodiments, the intrinsic reactances are substantially the same at each unit, with only the resistances Rᵢ varying. The inductance and capacitance per unit length can be estimated for example using microstrip impedance calculators.

In some embodiments, intrinsic reactances are deliberately varied along the longitudinal extent of the sensor (in which case ΔC and ΔL are replaced by ΔCᵢ and ΔLᵢ). Variation is be implemented, for example, by one or more of: the twist density (turns per unit length) of the wires, electrical insulation properties (e.g., insulating lacquer thickness), and/or conductor shape (e.g., tapering width, thickness and/or diameter; and/or longitudinally differentiated flattening of wires).

In some embodiments, the sensing material of sensor 310 comprises a thin, long resistive film located along resistive pathway 311 (e.g., interconnecting terminal 102 and terminating resistor Rₑ), optionally as a component of an FPC. Optionally, the film comprises a continuous extent comprising all of resistances Rᵢ, effectively arranged in electrical series. An electrical return path 310 (e.g., interconnecting Rₑ and terminal 103) is provided, for example, as a second wire and/or as a second layer of the FPC. Optional termination resistor Rₑ (optionally together with other components) is selected to match the characteristic impedance of the sensor.

As before, the total impedance of the sensor is measured through a frequency range, for example, between 1 MHz and 100 MHz. The sensor's sensing material (e.g., a resistive film) has a resistance per unit length that varies according to a magnitude, direction, or other quantity measuring a certain physical phenomenon.

For example, a continuous magnetoresistive film has a varying resistance per unit length along that film, the variation being an effect produced by a non-uniform magnetic field inside which the sensor is located. The sensor's self-inductance and self-capacitance are responsible for producing different impedance readings at each test frequency, and their differences are indicative of the continuous (but varying) resistance per unit length of the film, R(x):

$${Z}_{j} = {Z}_{tot} ( {ω}_{j} , R ( x ) )$$

In the above, Zⱼ is a function of the test frequency and the full resistance per unit length of the film, R(x), wherein x is the position along the film. Insofar as a finite number M of test frequencies is used for measuring the impedance, R(x) as defined cannot be solved to an arbitrary resolution; however, resolution constraints and/or other constraints/assumptions may be posed that allow finding potentially useful solutions. For example, R(x) can be assumed to vary smoothly along the film (for example, based on the assumption that the physical quantity being measured is smoothly varying in space and that the sensor's own shape is likewise smooth). The criteria for “vary smoothly” may be selected appropriately to conditions; for example, to exclude discontinuities above a certain magnitude in value and/or rate of change in value. Smoothness optionally imposes filtering constraints limiting the frequency-dependent amplitude of changes. Time-based constraints may be applied, e.g., to ensure that measurements near each other in time have correspondingly similar solutions. An earlier-determined solution may also provide an advantageous starting point for use with later-acquired measurements.

In more particular examples: R(x) is optionally modeled as a polynomial of finite degree N, or as a spline between N control points. These embodiments re-cast R(x) as a function of N variables Rᵢ. Again, Zⱼ can be represented using relations of the N “discrete” Rᵢ's.

Position Finding with a Magnetoresistive Sensor

Reference is now made to FIG. 4, which is a schematic flowchart describing a method of determining the position and shape of a flexible sensor within a set of generated magnetic fields, according to some embodiments of the present disclosure.

At block 401, in some embodiments, an EM transmitter is activated to transmit a well-characterized set of electromagnetic fields into a region, for example, a tracking region of a living body within which the position of a probe is to be determined and/or tracked. The electromagnetic fields are “well-characterized” in the sense that magnetic field vector and/or frequency is predictable for positions within the tracking region. To at least a first approximation, these properties can be determined on a theoretical (e.g., simulated) basis based on the known positions of field-generating magnets and their operating parameters. Optionally, calibration of any suitable type is performed to account for influences on the field due to other aspects of the environment. The aspects optionally include, for example, metals in known positions relative to the transmitter and/or sensor, properties of the tissue of the living body, stray magnetic fields, other nearby objects.

At block 403, in some embodiments, a probe comprising a longitudinally extended sensor comprising a magnetoresistive sensing material is introduced into the region of the living body through which the electromagnetic fields extend. Blocks 401 and 403 may be initiated in any suitable order. The introduction may be, e.g., as a guidewire leading a catheter, as part of a catheter, or as part of a longitudinally extended microsurgical tool of another type.

At block 405, in some embodiments, multispectral measurements are obtained using the sensor probe. The EM fields transmitted into the navigation region in themselves create a multispectral condition. The measurements optionally comprise readout of the resistance changes they create at any single suitable frequency. Optionally readout uses a DC signal. Alternatively, in some embodiments, readout comprises sequential and/or simultaneous excitements of the sensor circuit with a plurality of readout frequencies (e.g., 10, 20, 30, 40 or more frequencies) from within the range, for example of 1-100 MHz; for example as described in relation to FIG. 1. Optionally, readout frequency signal and EM field frequency signals are coordinated (e.g., synchronized to known phases).

Measurements are provided at block 408.

At block 407, in some embodiments, the measurements 408 and other information (e.g., constraint and/or calibration data 409) are used to find the sensor's curved position (and shape) in space. By the term “curved position” is meant a collection of positions along the longitudinal extent of the sensor, which together specify a shape of the sensor (potentially and likely curved in some degree), as well as an orientation and overall position (e.g., mid-point position) of that shape. The shape and position may be constrained, for example, by an anatomical track the sensor follows through the region of the living body, e.g., a track defined by vasculature, a portion of the gastrointestinal tract, and/or spaces between organs.

As before, total impedance of the sensor is measured at a frequency range, for example, between 1 MHz and 100 MHz. The sensor's resistive film has a varying resistance per unit length, due to the varying effect of the external physical quantity applied on that film. For example, a long magnetoresistive continuous film has a varying resistance per unit length along that film due to the effect of an external non-uniform magnetic field inside which the sensor is located. As before, the sensor's self-inductance and self-capacitance are responsible for producing different impedance reading per each test frequency, which are indicative of the now continuous resistance per unit length of the film, R(x):

$${Z}_{j} = {Z}_{tot} ( {ω}_{j} , R ( x ) )$$

That is, Zⱼ is now a function of the test frequency and the full resistance per unit length of the film, R(x) where x is the position along the film. Since possibly only a finite number M of test frequencies is being used for measuring the impedance, additional constraints are posed to allow solving R(x).

For example, R(x) is assumed to be smooth along the film (for example, based on the assumption that the physical quantity being measured is smooth in space and that the sensor's curve is smooth). In this case, R(x) is optionally modeled, for example, as a polynomial of finite degree N, or as a spline between N control points. In the latter case, R(x) is again a function of N variables Rᵢ, and Zⱼ can again be represented using relations of the N “discrete” Rᵢ's.

Instead of posing just a smoothness constraint on the physical quantity along the sensor's curve, further constraints may be used which are stricter and can therefore aid the solver converge faster and to a more accurate result. For example, a magnetoresistive curve sensor is usually placed inside known magnetic fields generated by a controlled EM fields generator. In this case, the magnetic field at each point in space is known exactly at each moment in time. Synchronizing between the transmitter and the curve sensor, the magnetic field is known at any position along the sensor's curve at any moment, assuming that the sensor's curve position in space is known. Rather than explicitly searching for R(x) that yields the impedance measurements Zⱼ, the problem then becomes finding the sensor's curve position in space at any point in time, such that the known magnetic field along that curve yields the observations Zⱼ. Formally, Zⱼ is written as a function of the sensor's position:

$${Z}_{j} = {Z}_{tot} ( {ω}_{j} , r ( x ) )$$

Where r(x) is the position of the sensor curve in 3-dimensional space, relative to the transmitter. As before, r(x) can be assumed to be smooth and can be described with N discrete positions rᵢ, between which a spline is fitted. It can also be assumed to have a fixed and known length at any point in time. Finding the curve's position in space doesn't necessarily allow for an arbitrary resistance function R(x) to be solved, but the function may be soluble for a continuous resistance which is observable inside the known transmitted field and under smoothness constraints of the sensor curve. This puts a stronger constraint on the solved resistance function $R(x). which improves the solver's speed and accuracy.

By whichever method is used, task of finding solutions is aided by numerous available or potentially available constraints, represented in aggregated by block 409. Such constraints optionally include, for example, any of: limitations on the plausible minimum radius of curvature of the sensor, the fixed and known distance along the sensor between each location x, previous known positions of the sensor in space, limits on how the sensor can move along its track (e.g., advancing and withdrawing along its already established path of introduction), and/or prior anatomical knowledge (e.g., a previous static image of the anatomy along which the device is navigating).

### Calibration

Solving for Rᵢ from the observations Zⱼ makes use of the idea that all the other characteristics of the circuit are known, or at least that discrepancies can be ignored. For a discrete-component sensor this means that Lᵢ, Cᵢ are known. For a continuous sensor this means that the impedance and capacitance per unit length (and so also the characteristic impedance) are known. If by some manipulation these also vary per unit length, then the function by which they vary is also known.

In reality, electrical components have tolerances, and potentially even environmental sensitivities of their own, so that their exact impedances may not be precisely known simply based on their specified values. Also (particularly in the case of discrete-component sensor embodiments) self-capacitance and self-inductance of the circuit may add “virtual” Lᵢ, Cᵢ. A portion of these variables may be accounted for through calibration (e.g., empirical investigation to detect and/or characterize them).

As an example of calibration: for a magnetoresistive sensor, a constrained calibration process is used in some embodiments, based on methods similar to those described above.

The sensor may be placed within a known transmitted magnetic field and its shape and position may be deformed by an operator, optionally in an unsupervised manner. Measurements in the no-field case may also be performed. At each moment in time, Zⱼ are sampled, and these measurements are indicative of the Rᵢ's. However, more generally, Zⱼ are also indicative of the other circuit's characteristics: Lᵢ, Cᵢ. Under the constraints of a known transmitted magnetic field, plus curve smoothness and/or distance constraints, all of Rᵢ, Lᵢ, Cᵢ can be simultaneously solved in an optimization process (and not just Rᵢ as before). During calibration, there is potentially a lowered requirement for real-time performance, so the calibration solver can optionally configured to run to a higher level of convergence, and/or using a reduced number of constraints and/or approximations.

This calibration process may be performed for each sensor, e.g., factory performed as part of their production. As an optional constraint on calibration calculations, Lᵢ, Cᵢ may be assumed to have fixed values for all the measurements over time, while Rᵢ changes per timestamp (since the sensor moves). For the correct values of Lᵢ and Cᵢ, substantially all the constraints will be met over substantially all timestamps (within the limitations of sampling error and other noise): that is, that the sensed magnetic fields (derived from Rᵢ) will conform to the known transmitter field, and the solved sensor curve positions (derived from the magnetic fields) will be smooth. In addition, computing the desired physical quantity from resistance measurements Rᵢ relies on accurate translation between resistance measurements and the desired physical quantity (e.g., magnetic field, temperature). Resistance-to-physical quantity curves for each resistance Rᵢ may be calibrated as part of the same process.

In an alternative calibration process, a more supervised method can be used. In case of a sensor using magnetoresistive sensing material, the sensor is placed to extend through positions in space whereat the magnetic field is fully known. Possible values of Lᵢ, Cᵢ are then searched so that the solved magnetic fields will equal the target fields at these positions. For a thermo-resistive shape sensor, the sensor can be placed inside a special device which sets known temperatures at known positions along the sensor's curve. This provides the target measurements for the sensor. The circuit characteristics (i.e., Lᵢ, Cᵢ) can then be searched such that the finally computed Rᵢ will yield the target temperatures.

It may be noted also that providing certain combinations of sensing material—or even the same material in different orientations—may be of benefit in isolating desired sensing responses, compared to other resistive change which may occur.

For example, magnetoresistive materials may be anisotropic-responding more to fields oriented in one direction than in another. This could lead to “dead zones” for certain fields in certain locations where the orientation of the sensing material leads to insensitivity. In some embodiments, the sensing material is wrapped or twisted around the longitudinal extent of the sensor, and/or twisted on itself, and conditions and/or processing of measurements chosen in such a way that measured resistances are binned to combine sensing material in different orientations. Additionally or alternatively, local differences in material orientation are used to inform the system about sensor state. For example, as the sensor becomes twisted, two nearby portions of anisotropic magnetoresistive material may move relatively into or out of alignment in their orientations. Changes in the relative directionality of the magnetic fields they are sensitive to thereby becomes indicative of twisting of the device.

In another example, temperature response may be the same no matter what the orientation of a sensing material is, but strain sensitivity may be anisotropic. This may allow parasitic temperature responses to be isolated from strain responses, and potentially even allow even the same sensing material to be used for distinguishably measuring two or more environmental parameters.

### Endoluminal Navigation-Related Features and Applications

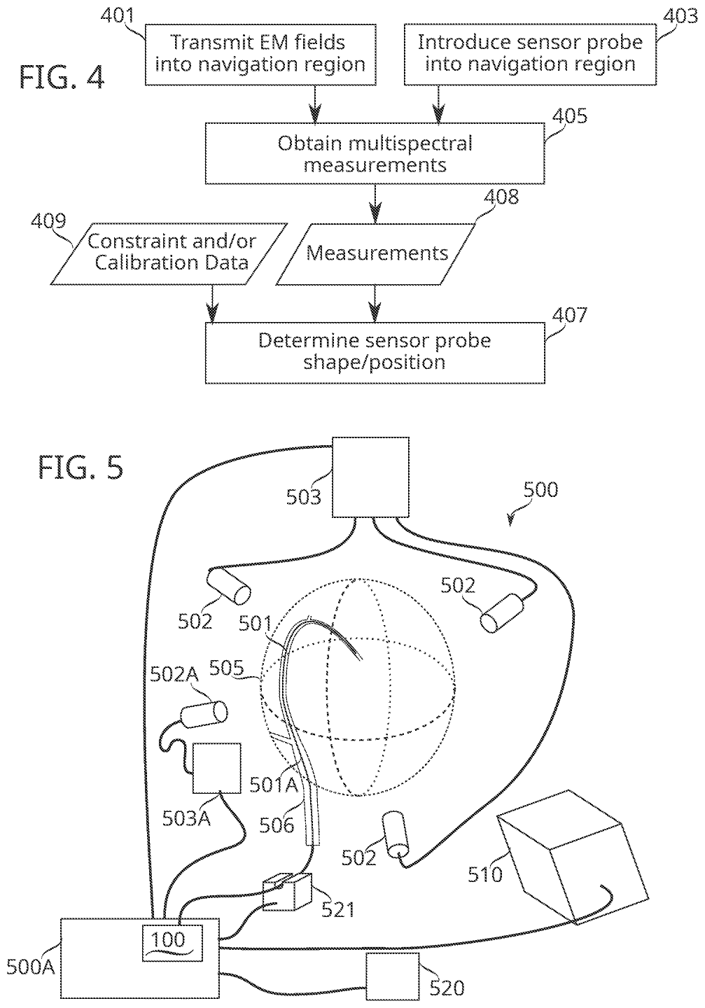

Reference is now made to FIG. 5, which schematically represents an endovascular tracking system 500, according to some embodiments of the present disclosure.

Sensor 501 is a spectrally multiplexed, longitudinally extended variable resistivity sensor, corresponding, for example, to one or more of the embodiments of FIGS. 1, 3, 6, 7, or another spectrally multiplexed, variable resistivity sensor. Sensor 501 is interconnected with external processing unit 500A and/or external processing unit 100 (e.g., via two or more connection terminals 102, 103, not shown). External processing unit 500A may fully incorporate external processing unit 100 (including, in particular, its function as a readout controller), and optionally provides other processing features, for example as described in relation to embodiments corresponding to FIG. 5, or other embodiments herein. In some embodiments, the functionality of a readout controller is provided separately from most or all data processing functions, i.e., separate from external processing unit 500A, but in communication with it so as to transmit measured data, and optionally to receive commands governing how sensor 501 is controlled and/or how measurements are collected from it. Readout controller functions include at least driving sensor 501 with electrical current used in magnetic field sensing. Moreover, readout controller 500B senses the magnetic field-reactive state of sensor 141, via the influence of its (variable) resistance on properties of the electrical current (for example, its oscillation frequency and/or amplitude of oscillation at one or more frequencies). External processing unit 500A may incorporate additional processing features. For example, it may perform integration of measurements made using sensor 501 with sensor data from another modality such as imaging device 510, or any additional sensors which may be provided as part of navigable probe 501A. In some embodiments, processing unit 500A processes according to inputs received from user interface 520 (e.g., according to selected views and/or operational modes), and/or performs generation of displays for user interface 520.

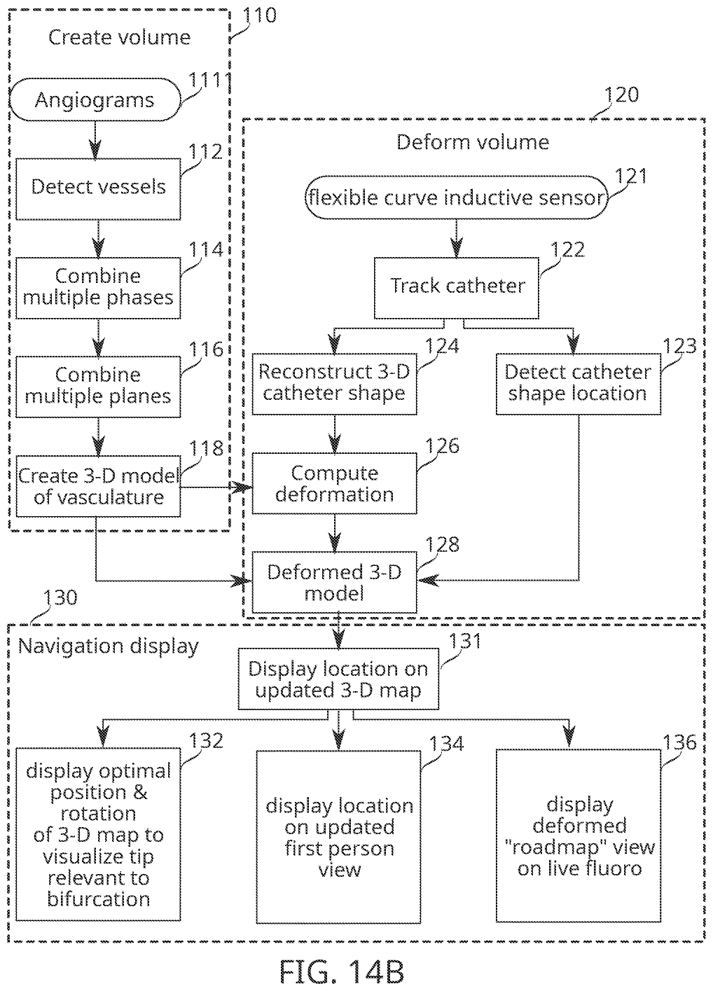

It should be understood that features described in relation to the system of FIG. 5 which are not directly dependent on magnetoresistivity per se are optionally provided in systems using another form of shape tracking sensor, for example, a magneto-inductive sensor, e.g., as described in relation to the embodiments of FIGS. 9-11. Similarly, features of FIG. 5 are combined freely in some embodiments with features of systems according to FIGS. 14A-14B, insofar as they are compatible; e.g., insofar as they do not inherently depend on a particular sensor type (e.g., operating principle). In some embodiments, different devices having different sensor types (e.g., magnetoresistive and magneto-inductive) are used within a same system provided with features described in relation to any of FIGS. 5 and 14A-14B.

In some embodiments, a single device may be itself provided with a plurality of curve-sensing types (e.g., both magnetoresistive and magneto-inductive); along different regions of its length and/or along shared portions of its length. The extra data provides potential advantages, for example, in assisting the discovery of shape solutions, albeit potentially at the cost of complexity and/or device size. A magneto-inductive sensor is optionally provided together on a device with an additional sensor type described here particularly in relation to combination with magnetoresistive sensing; for example, one or more piezoelectric and/or thermoresistive sensors. Similarly, modes of navigation and/or navigation assistance described with respect to these additional sensor types are optionally provided together with systems providing magneto-inductive sensing for navigation.

Also provided, in some embodiments, are EM field control unit 503, interconnected with a plurality of field generators 502 (e.g., magnetic field generators). Field generators 502 are arranged around navigational region of interest 505, comprising, for example, a portion of a human body (e.g., a cranium).

Sensor 501, as part of a navigable probe 501A, is shown introduced into navigational region of interest 505, via a vasculature 506, of which a restricted portion is shown for purposes of illustration.

Among the potential advantages of a variable resistance-based sensor 501 with spectrally multiplexed readout is its potentially very small cross-section; potentially as small as can be realized by a pair of wire conductors. In some embodiments, the sensor 501 is suitable for use in a probe 501A with a maximum cross-sectional diameter of 1 mm or less, 750 μm or less, 500 μm or less, 400 μm or less, 350 μm or less, 250 μm or less, or less than another cross-sectional diameter. The probe may be integrated into a guidewire, or other longitudinally extended tool suitable for insertion into longitudinally navigated anatomical channels such as blood vessels and/or airways. Additionally, the probe can serve as a guidewire, sharing both electromagnetic and mechanical properties (e.g., pushability, torqueability etc.) needed for a guidewire, while being a magnetoresistive or magneto-inductive sensor as described herein.

Another potential benefit of such sensors, particularly but not exclusively in a twisted wire pair configuration is design simplicity and cost, with corresponding potential advantages for use as a disposable device. For example, in certain applications a plurality of magnetoresistive fully tracked “guidewires” can be introduced to a patient's organ and provide a fully tracked real-time skeleton of that organ for certain uses, such as modeling the deformation of that organ during a medical procedure.

The most distal portions of the sensor 501 are optionally constructed thinner than more proximal portions. This allows tradeoffs, e.g., such as adding discrete components in larger-diameter regions, further differentiating them electrically from the electrical properties of smaller-diameter regions where volume to add discrete components is more restricted, or absent.

Insofar as it is common for guidewires to comprise metal portions to achieve appropriate levels of stiffness and/or pushability, some portion of the sensor circuit (especially but not limited to the signal return conductor) may have double functions both mechanically and electrically.

Within the context of a minimally invasive medical procedure relying on endoluminal (e.g., endovascular, or more particularly, neurovascular or cardiovascular) guidewire navigation, there may be more than one sensing modality in place. For example, external imaging device 510 is optionally provided, which is optionally an X-ray imager. Ionizing radiation-based imaging, while well-known and commonly used as a way of monitoring endovascular instrument positioning, has associated exposure risks. In some embodiments of the present disclosure, position determination using a variably resistive sensor probe 501A is used to allow reduction of ionizing radiation exposure, e.g., reduced imaging frame rates (e.g., 5 Hz instead of 30 Hz, or another factor of frame rate reduction). To maintain an acceptable level real-time feedback, interpolation of probe position through the “missing” frames optionally uses spectrally multiplexed position sensing to infer changes in position, e.g., from the previous X-ray “key frame” image. Sensor-inferred positions of the sensor-equipped guidewire are shown to a surgeon for the period between each directly imaged positions, to help maintain their positional awareness.

Each new X-ray key frame also optionally serves as a check on the accuracy and calibration of sensor data-based position calculations, optionally implemented by external processing unit 500. Optionally, adjustments are made to (for example, calibration is corrected for) the next round of position calculations which would have corrected errors noted during the previous round. Additionally or alternatively, the frame rate of ionizing radiation imaging is adjusted (e.g., via communication between external processing unit 500 and imaging device 510) according to the amount of error accumulated during the interpolation stage. If error is small, the frame rate may optionally be decreased, and if it is large, the frame rate may optionally be increased. This provides a potential advantage for maintaining a preferred tradeoff between exposure risks and reliance on more indirect methods of device tracking with the potential for gradual error accumulation. Whatever the sources of the position information at any given moment, they are optionally integrated by external processing device 500, and provided (e.g., as a live-updating display) to user interface 520. The live updating display may indicate overall context of the field of device navigation (e.g., in the form of a recently acquired X-ray image from imager 510, or another image, for example as may be obtained in the form of a CT scan or MRI image).

Additionally or alternatively, the sensor 501 itself may incorporate more than one type of sensing material. For example, it may comprise both magnetoresistive and piezoresistive material. The piezoresistive inputs potentially allow assessing sensor curvature (e.g., with more stress or strain corresponding to greater curvature), placing further constraints on overall position finding. Piezoresistive material optionally comprises a separate (e.g., parallel-wired) channel from the magnetoresistive material, and/or portions of piezoresistive material may be wired in series with magnetoresistive material.

A potential complication of endovascular navigation, particularly within increasingly fine vasculature, is the risk of perforation. Perforation in turn may become more likely as forces on the instrument being navigated (e.g., a guidewire) increase. This is another use case for piezoresistive material (which may be configured to be sensitive to such forces), optionally provided on sensor 501, in some embodiments, with or without the concomitant provision of magnetoresistive-based navigation capabilities.

It may be noted that through a bend, there are potentially both compressive and tension forces experienced simultaneously. Along a longitudinal extent of a tool, a strip of sensing material may be wound so that it alternatively extends for brief distances on all of its sides. This potentially helps to distinguish directions of flexure and twisting through different degrees of freedom (e.g., up/down, left/right, and twisted).

Potentially, wall contact information (e.g., contact with a wall of vasculature 506) is generated from environmental sensing, e.g., by noting the pattern of strain build up along a sensor. It may become clear, for example, that regions proximal to some longitudinal position of probe 501 are under stress, while regions distal to it are not. This is potentially indicative of the instrument exerting potentially damaging forces at a position away from the distal-most tip itself.

In blood vessels 506, thermoresistive sensing may be used to investigate flow conditions surrounding particular portions of the probe. For example, inducing mild resistive heating into the probe warms it up, potentially at a heating rate and/or up to a temperature which is affected by how well heat is being carried away from the probe. This may in turn be affected by a cooling effect (or lack thereof) dependent on surrounding flow. The rate of return to baseline temperature potentially is similarly affected. Flow conditions may in turn be indicative, e.g., of which side of a blood vessel 506 a probe 501A carrying the sensor 501 is pressed up to. In some embodiments, flow is also important in endovascular procedures, for example, in neurovascular procedures, to assess the blockage condition of certain blood vessels, for example in clot retrieval procedures, or while deploying balloons inside blood vessels, or for other type of diagnostic or therapeutic endovascular procedures. It is also potentially advantageous to construct a flow map of blood vessels, that is, to be able to measure and visualize the flow inside different blood vessels in different 3D positions. To do that, the tracked position and shape of sensor 501 can be used, along with a flow measuring technique, as described above, to assign a flow measure (which can be a scalar measure or a full 3D flow vector) to one or more positions in space, inside the anatomy. This may enable constructing a 3D flow map of a patient's blood vessel system, such as the neurovascular system.

With the availability of a rich dataset of shape, position, and/or contact-related sensing data from all along the length of a probe, there are potential advantages for automatically assisted navigation of a probe. For example, there may in general be available a plurality of degrees of freedom used during the advance of a longitudinally extended probe 501A through a restricted lumen such as vasculature 506—forward/backward, twisting, and optionally steering with one or more degrees of freedom. In some embodiments, selection of an optimal order and/or amount of actuation of these degrees of freedom is informed by how the probe 501A itself responds to inputs, based on this sensing. For example, there may be a preference to select in each moment the most “slippery” motion—the one that best minimizes overall strains on the device while still allowing it to advance forward. In some embodiments, for example, small manual or automated trial motions are attempted, each receiving rapid feedback from along the probe's longitudinal extent. Motions actuated by the degree of freedom with the recent “best” performance may be noted and optionally recommended (e.g., on a display). In automatic embodiments, the movements may be executed, amplified, and/or repeated, and the process of testing repeated. Tracking the shape of a probe is also potentially advantageous for driving an endovascular, or more generally, endoluminal probe, whether manually or robotically. Using the tracked shape of the probe as feedback, the steering of the probe can be made more efficient in an attempt to drive the probe from an origin to some destination. For example, when pushing the probe, the shape of the probe may tell how the push action affected the probe and whether the probe did advance in response to the driving action, or whether, for example, a loop has formed along the shape of the probe, in which case the probe may need to be pulled back. This kind of visual shape feedback, along with others, can be used manually by a physician holding the probe and manipulating it (for example, a guidewire), or by a physician holding a remote controller for a robotic driving mechanism which drives the probe, or for a semi-automatic or a fully-automatic robotic driving mechanism, which may use the probes real-time tracked shape, as the physician would, to realize how steering actions are acting on the final shape of the manipulated probe.

Another method of variable resistance-based navigation may be implemented based on thermoresistive properties. Methods exist for remotely inducing focal heating; for example, high-intensity focused ultrasound, or another form of energy which can be focused using lensing and/or phased array methods. The heating energy need not be elevated to destructive levels, however. Instead, it can be focused to create one or more mild hot spots, different enough from the surroundings to generate thermoresistive changes. The hot spots may be scanned (e.g., by operation of transducer 502A under the control of transducer controller 503A) in the region of a probe to determine its position (the probe gets hot where and at the moment that the energy focus intersects it), and/or moved along a preferred path of the probe (and slightly in advance of the probe itself) to “lead” the probe toward a preferred position. This type of “tropism” (end-seeking) navigation method may be suitable for adaptation to automated control methods, by setting up a clear navigational signal as a gradient that indicates the desired direction of movement, and provides a readily measurable parameter for evaluating progress.

It may be noted, for example, that the longitudinal extent of the sensor 501 provides a large target for the energy focus to find some portion of, and then a pathway that allows guiding movement of the focus from that portion to the distal end of the sensor. From that state, the energy focus can then be switched over to the role of leading. A correct direction and safe distance to next move the energy focus can be verified, for example, by intermittent imaging, there being periods in between image taking wherein the advancing probe is monitored through sensing as it catches up to the focus. If the probe (which can be seen by the imaging) is known to be at the focus, then the focus itself can be moved by a known amount judged from the image, and the probe again moved until it reaches the focus.

A similar type of leading navigation is potentially available with other manipulations and sensing. For example, suitably focused EM fields generated by transducer 502A (this time as a magnetic field generator) and a magnetoresistive sensing material on probe 501A may be used. A probe 501A may, for example, be navigated to seek a field region having a certain phase, direction, and/or magnitude in a gradient established by a movable electromagnetic field generated from transducer 502A, optionally without necessarily knowing exactly where the probe is at every moment, but still being certain from real time measurements that the target is being approached, without being overreached.

In some embodiments, an optional robotic driver 521 is provided, configured to manipulate probe 501A under the control of processing unit 500A. For example, probe 501A is manipulated with respect to one or more of: its distance of longitudinal advance, rotation about its longitudinal axis, and its steering articulation angle. Optionally, control over one or more additional degrees of freedom is provided, e.g., for use in tool actuation, and/or additional steering degrees of freedom.

Control, in some embodiments, is guided according to sensor data, and in particular according to the sensed shape and/or position of sensor 501. Optionally, data from additional sensing modalities (e.g., from imaging device 510) are integrated, e.g., in order to assist referencing of sensed shape and position to a current anatomical state of the region being navigated (e.g., relative position of a navigational target such as a branch of a bifurcation). In some embodiments, robotic control is provided in the form of navigational assistance for certain tasks. For example, robotic control may be activated for assistance in steering tasks and/or passing obstacles, with navigational guidance overall being apportioned to manual operations.

In some embodiments, robotic control is used to help in manual selection of movements. Optionally, manual control of probe 501A is performed through controlling actuators of robotic driver 521 itself. Optionally or additionally, robotic driver 521 may be configured to receive indications of forces exerted manually on probe 501A from outside of its control, e.g., it may sense changes in advance or rotation, and or sense tension and/or compression inn probe 501.

In some embodiments, the assistance is in the form of governing the dynamic characteristics of the device in response to inputs; e.g., reducing sensitivity to steering inputs when it is judged (automatically, and/or in response to a user input) that the operator is and/or is expected to be searching for a steering angle within a small range. Automatic judgements may depend, for example, on the recent history of user adjustments (e.g., an oscillating input may indicate difficulty in finding a needed steering setting). Additionally or alternatively, the judgement may depend on a recent history of sensor shapes and/or positions, where again, oscillation may indicate a potential difficulty. In some embodiments, sensor-observed mismatch between attempted inputs and observed positions of sensor 501 is used to help identify situations in which assistance may be useful.

In some embodiments, the assistance comprises executing a plurality of micro-movements which help the robotic system to assess the current state of the device, and which inputs may assist further progress toward a goal such as a target position. Effects of the micro-movements are optionally measured based on assessment of the shape and/or position of sensor 501. For example, a distal tip of sensor 501 may move or not move; if it moves, it may move in an intended direction, or not. Changes to the shape of sensor 501 may indicate how manipulation forces are being absorbed. For example, upon receiving an advancing input, increased bending of sensor 501 up to but not past a certain location may indicate that there resistance to sliding motion concentrated on that location. Such findings may be indicated to the operator, e.g., via user interface 520.

In another example, changes to the shape of sensor 501 in response to micro-movements may help determine combinations of inputs which may be helpful. For example, deflections in the shape of sensor 501 along its length may change slightly as small movements (e.g., advancing/retracting and/or twisting) are made which adjust forces and/or points of contact. Adjustments which tend to move the sensor 501 toward a more favorable starting point for receiving further inputs like steering angulation are optionally detected. The operator may choose, for example, to carry out those adjustments themselves (upon receiving suitable indications, e.g., via user interface 520), or to have the robotic driver 521 itself amplify promising adjustments into larger movements.

### Sensor Designs

### FPC Sensor Design

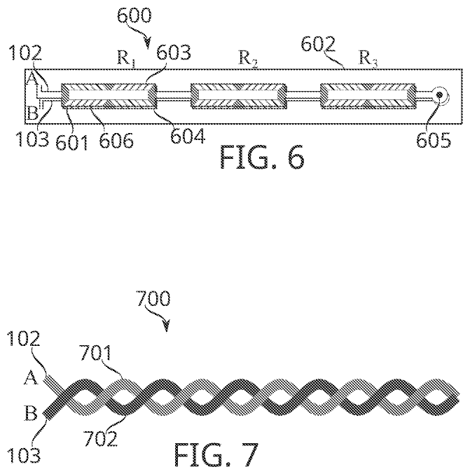

Reference is now made to FIG. 6, which schematically represents a two layer sensor FPC with discrete “barber pole” resistive elements 603, according to some embodiments of the present disclosure. In some embodiments, the “barber pole” arrangement comprises a plurality of substantially parallel diagonal stripes of magnetoresistive material 601, joined through inter-spaced conductors 606 connecting one stripe to another at an oblique angle (e.g., 45 degrees) relative to the longitudinal axis of the FPC. This configuration forces an obliquely oriented electrical current between the resistive stripes. This potentially exploits anisotropic properties of a magnetoresistive film to promote sensing of fields with orientations away from, e.g., a longitudinal axis of the probe.

In some embodiments, a sensor 600 comprises sensing material 601 arranged in discrete units 603, optionally without any introducing discrete reactive components in between. In the example shown, sensing material 601 is encapsulated by a flexible printed circuit (FPC) ribbon 602.

A potential advantage of breaking the sensing material 601 (e.g., material in the form of a film) into discrete units is focusing resistances into more distinctly localized locations along the longitudinal extent of the sensor 600.

This potentially makes sensor impedances easier to determine from measurement data; e.g., the approximation that they can treated as N discrete resistances Rᵢ more closely reflects the physical arrangement of the device. This may make the resistances Rᵢ easier to determine, compared to the case of a continuous and homogeneously distributed resistance and/or resistance function R(x).

In some embodiments, sensor 600 comprises a two layer FPC circuit, having a plurality (e.g., N=3 as shown, but optionally more, for example, 10, 20, 30 or more) “barber pole” discrete units 603. Discrete units 603 are provided as traces comprising sensing material 601, optionally in the form of a resistive film (shown as the top gray layer).

The second (bottom) layer, in some embodiments, contains a signal return path 604. This can be made of more units 603 (e.g., as shown) or of an ordinary conductor such as copper, and/or of a sensing material arranged in another fashion. That may be of the same, or optionally of a different sensing material, although in the case of a different sensing material, it may be treated as an additional resistance, instead of as one of the resistances Rᵢ shown.

Connections are soldered or otherwise joined to the FPC at terminals 102, 103 (i.e., pads A, B). The layers may be connected at the distal end of the FPC using a Via 605. A termination resistor Rₑ or other termination element (not shown) may be added to the circuit just before the Via.

In some embodiments, FPC sensor 600 contains a special layer which may be made of special resistive material (for example, a magnetoresistive material) instead of plain copper. This material can be incorporated in the FPC manufacturing process similarly to the inclusion of a standard copper layer, and may undergo the same manufacturing processes as copper (such as etching). This allows the resistive material to be shaped and laid out precisely along FPC ribbon 602.

In some embodiments, sensor FPC is wound helically around a cylindrically structured device (e.g., a pipe-structured device such as a catheter). The configuration can be made such that each consecutive resistive element (which amounts to a sample point in space of the magnetic field) sits on a different side of the pipe, such that the elements units are oriented in at least two different orthogonal directions along the pipe. Since, in embodiments using EM sensing, a magnetoresistive FPC sensor may have anisotropic sensitivity to field direction, this potentially makes the magnetic measurements more informative, assisting determination of the sensor's position over its full longitudinal extent. In such configuration, it might be important to know the exact geometry of the sensor winding around the pipe. This geometry can be determined in supervised or unsupervised calibration processes, by measurement, by manufacturing process design, or another method.

Insofar as measurements may be invariant to rotation of the FPC about the FPC's length axis (although, as described hereinabove, they may actually be sensitive to this, depending on device configuration), it may not be practical to solve the twist of the curve in space using them, although this information may be useful for some applications.

In some embodiments, accordingly, an additional resistive film layer may be added to the FPC, optionally identical to the first layer but with an offset, e.g., to an opposite side of the sensor. Sensor measurements are made from each layer, and positions computed for them both. When the FPC is twisted, the twist is potentially noticeable as a discrepancy between the two solved curves. The final catheter position (and twist) can be solved by combining the two solve position curves and adding the difference vector between them, which determines the positional twist of the curve. Another method of detecting device twist is described, for example, in relation to FIG. 4, herein.

### Twisted Pair Sensor

Reference is now made to FIG. 7, which schematically illustrates a twisted-pair variable resistive sensor configuration, according to some embodiments of the present disclosure.

In some embodiments, a sensor 700 comprises electrically isolated variably resistive wire 701 may be used which is made of material similar to that of the variably resistive film (for example, enameled wire). The variably resistive wire 701 may be twisted to create a twisted pair between the wire 701 and another wire 702. Wire 702 is optionally itself variably resistive, or made of a plain conductor.

This creates a circuit similar to that of, for example, FIGS. 1 and 3. The two wire portions 701, 702 are inductively and capacitively coupled and optionally terminated with a Rₑ resistor or other terminating element. The twisted pair characteristic impedance can be calculated using known formulas, and the continuous resistance R(x) can be computed from total impedance measurements Zⱼ using the methods described above. The construction of such sensor is relatively simple and its footprint is potentially ultra-small (for example, smaller than 0.5 mm, smaller than 360 μm or about 0.014 inches, smaller than 250 μm, or another diameter). The number of twists in the self-twisted pair affects the characteristic inductance of the circuit and can be changed according to the application. It (twisting) may be non-uniform along the length of sensor 700. In the extreme case, the wire may not be twisted at all, in which case the two wire portions 701, 702 may extend one next to the other in parallel.

In some embodiments, reactance (e.g. inductance and/or capacitance) differences arising from varied twist density as function of position along the sensor are used to help electrically differentiate different regions, so that alterations in resistance along the sensor are distinguishable, e.g., as described in more generally in relation to FIG. 3 with respect to the variation of intrinsic reactances along the sensor length. In some embodiments, conductive material used in one or both strands of the twisted pair is selected for its mechanical properties, e.g., its steerability, torqueability and/or pushability. More magnetoresistive and more mechanically stiff portions of the twisted pair may alternate, optionally with the other member of the pair being constructed to provide the complementary set of characteristics along same respective extents of the sensor. In some embodiments, the material of at least portions of one or both of the twisted pair members is selected for its properties as a magnetic core, e.g., a high-permeability non-linear material; made for example, of permalloy, supermalloy, mu-metal, cobalt alloy, or any other high permeability non-linear magnetic core. This is optionally arranged to enhance changes in circuit resonance characteristics as a function of magnetic field strength.

Twisting by the members of the twisted pair is not necessarily symmetrical. For example, one strand may be thick enough that the other strand winds around it, while the thicker strand deflects by smaller amounts per twist, or not at all. The asymmetry may approach or actually provide the geometry of an inductor coil having a straight internal core, and a wound coil around it. The magnetoresistive material is optionally provided as part of the more coil-like portion, and/or as part of the more core-like portion. In some embodiments, which strand of a pair is thicker and which is thinner is the same all the way along the probe. In some embodiments, the strands alternate construction along their lengths.

Optionally, the more core-like portion is twisted with twists that go around itself, rather than around its partner lead. For example, a film-like magnetoresistive material itself may be constructed in a fashion (e.g., as a layered film, with limited ductility, and/or having a width notably larger than its thickness) which is difficult to wind as a coil layer with a pitch small enough to make appreciable inductance contributions. As may be appropriate to keep its maximum cross-sectional dimension small, the material may optionally be twisted with a relative long pitch, optionally surrounding a supporting material. The supporting material's properties as a magnetic core may be high-permeability and non-linear, or otherwise.

Material provided for a thinner strand of an asymmetrically constructed twisted pair may be suitable to be wound into a fine-pitch coil (e.g., as thin copper or gold wire, e.g., in the range of 40-54 AWG). Optionally, it is wound along the sensor in a pitch that is different in different sections (continuously or variably), and/or in different numbers of layers in different sections. This provides reactance variation which may be used to help distinguish resistivity changes in different magnetoresistive sections along the probe.

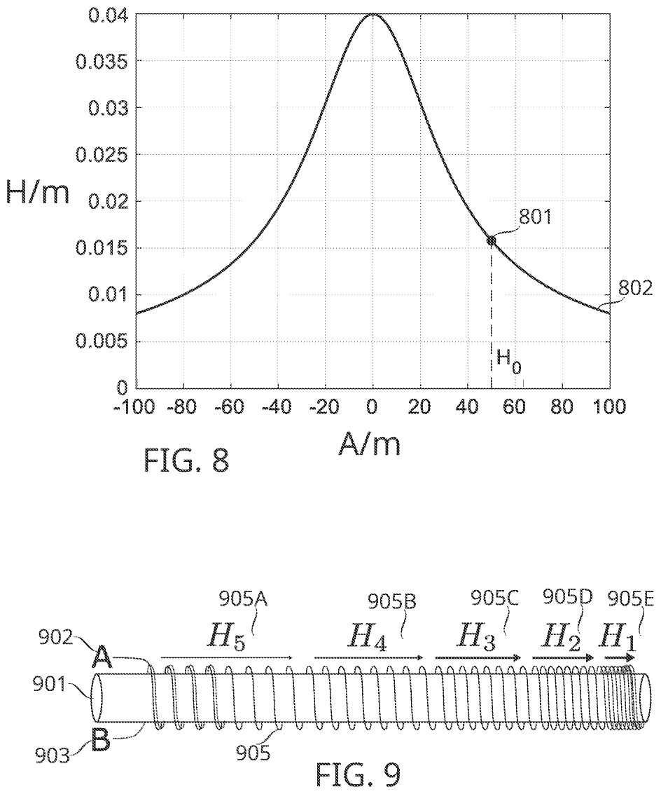

Methods of spectral decomposition analysis described with respect to magnetoresistive sensing are based, in some embodiments, on how local circuit resonance properties change along a sensor's length according to interactions with a magnetic field. In magnetoresistive sensing, these resonance property changes are the result of resistance changes, with the spectral decomposition being supported by an overall inhomogeneity in baseline resonance properties along the sensor's length, e.g., as determined by resistance, inductance, and/or capacitance. In some embodiments, magnetoresistance changes are supplemented, or optionally replaced, by magnetic field interactions with another electrical parameter of the sensor. For example, the use of magnetoinductive properties as the basis of magnetic field sensing are described with relation to the embodiments of FIGS. 8-14. In relation to those figures, a method of analyzing measurements is described which uses currents to generate location-dependent offsets (e.g., from a zero-magnetizing field baseline condition) of the permeability of high-permeability non-linear core materials. However, it should be understood that there is also an option, used in some embodiments, to apply the spectral decomposition method described for magnetoresistive sensor probe embodiments using magnetoinductive sensor probe embodiments additionally and/or instead. For example, the various inductances L₁ . . . Lₙ of FIG. 1 and/or ΔL of FIG. 3 may include a magnetoresistive contribution (e.g., from an inductor with a highly permeable, non-linear core) that affects circuit resonance locally. Optionally, constructions described, e.g., in relation to the magnetoinductive proves FIGS. 9-11 and/or 13 are used in such embodiments.

As mentioned above, certain physical quantities affect the resistance of different type of films. However, sometimes a plurality of physical phenomena may affect the film, which is undesired if only a specific physical quantity is to be measured. For example, a magnetoresistive film may also be affected by temperature and strain which may affect the magnetic measurements as parasitic effects. In order to compensate for these effects, a symmetric (but non-magnetoresistive) layer of the same geometry as the magnetoresistive film may be added to an FPC sensor and its resistance can be measured at multiple frequencies using the same methods described above. Since the layer is not magnetoresistive it will only measure the parasitic effects: for example, thermo-resistive and strain. These measurements (suitably calibrated) can then be subtracted from the measurements of the magnetoresistive layer, thus compensating for the undesired parasitic effects and improving the magnetic measurements.

In a magnetoresistive sensor, the magnetoresistive film may be magnetized in order to be able to sense the magnetoresistive effect. To accomplish this, an external processing unit 100, 500 may send a high electrical current pulse through the curve sensor's ports and into the magnetoresistive films to magnetize the films. The pulse can be long enough (for example 100 μs long) such that it is almost considered as a DC current with which the reactive elements of the circuit need not interfere.

Reference is now made to FIG. 8, which plots a magnetization (or magnetic strength, in A/m) to permeability (or “inductance per meter” in H/m) relationship of an example magneto-inductive coil, wrapped around a high permeability non-linear magnetic core, according to some embodiments of the present disclosure.

In a magneto-inductive sensor according to some embodiments of the present disclosure, a high permeability non-linear magnetic core with an induction coil wrapped around it interacts with the magnetic field at points along the (typically curving) core's longitudinal axis. Current in the coil experiences inductance which changes according to the strength of the total magnetic field which the non-linear magnetic core material experiences. The actual “experienced strength” may be judged by the resulting effects on current, which potentially vary from effects expected from the magnetic field vector as such, due to factors such as anisotropic sensitivity of the core material. The change in inductance is due in turn to the dependence of a coil inductor on the magnetic field permeability of its core material.

Accordingly, suitable measurement of how current flows through the sensor provides an indication of the magnetic field's properties. In particular, by utilizing a pre-calibrated permeability to magnetic field relation, the magnetic field can be computed.

In order to get the maximum sensitivity from the magneto-inductive sensor, it may be measured around the linear regime of the inductance curve 802—that is, it is measured in a region where the change in permeability is relatively linear as a function of changing electromagnetic field strength (and more particularly, magnetization field strength). As shown in FIG. 8, this relatively linear regime is displaced to either side of zero field magnetization field strength. However, driving a current through the coil applies a bias magnetization H₀ (marked at graph location 801), which is preferably selected to be near the middle linear regime of the curve 802.

The sensor's inductance can then be measured, with shifts from the bias location 802 resulting in a change in permeability (vertical axis of FIG. 8) which can be referenced to the amount of added or decreased magnetic field strength in the environment (displacement from location 801 along the horizontal axis of FIG. 8). As implemented for some embodiments of the present disclosure, the relationship may be described in another way, for example, as being among two different but related parameters. For example, permeability in H/m may be replaced by reference to inductance per coil, or linear inductance density (also in H/m) as such. Magnetization in A/m may be referenced to current (at least when current induces it), or to another parameterization of the electromagnetic field such as the magnetic field vector.

The sensor's inductance is optionally measured using RLC oscillation methods, digital oscillation methods or any other suitable method. For example, in a digital oscillation inductance measurement method, a bias resistor R_{b} determines the electrical bias current (and therefore also the bias magnetization of the coil). The bias introduced by the inductance measurement itself may be determined by comparing to measurements in which the sensor is reverse biased and sensed around negative bias magnetization −H₀.

In some embodiments of the present disclosure, a flexible magnetic core is used so that the magneto-inductive sensor built around it can be flexible. For example, the magnetic core is constructed as a flexible thin wire (for example, 0.2 mm in diameter). The flexible core wire is made of a high permeability flexible material such as supermalloy. One example of a supermalloy is composed of nickel (75%), iron (20%), and molybdenum (5%). The one or more coils of the sensor are wrapped around the core wire. The conductor of the coil(s) comprises, for example, 38 AWG enameled copper or gold wire, or thinner (e.g., 40 AWG-54 AWG).

In the setting of a single long coil, as discussed above, the total inductance of the sensor can be measured and the total magnetic field along the sensor's flexible curve can be sensed. This readily provides a measurement of the average (e.g., algebraic average) magnetic field experienced by the whole sensor (though the average is potentially weighted somewhat due to non-linearities present in the system).

In some embodiments of the present disclosure, additional features are provided to enable distinguishing of the potentially different magnetic field values which exist along the sensor's curve. The magnetic core's non-linearity is exploited in order to decompose the sensor's inductance measurements into different inductances along respectively different portions of its length.

Reference is now made to FIG. 9, which schematically represents an inductive sensor comprising 5 discrete coils connected in series, according to some embodiments of the present disclosure. Due to the coils' different pitches, each coil exerts a different magnetization force on the segment of magnetic core 901 which it surrounds.

The 5 discretely labeled inductive elements 905A-905E of FIG. 9 (labeled also with their associated magnetization forces H₁ to H₅) comprise coils wrapped around a high permeability non-linear magnetic core 901, in increasing winding pitches, and connected in series. In the example as depicted, the coils are wrapped with wire 905 from left to right, and then wrapped in a second layer from right to left (indicated by the dotted lines) so that the two sensor's coil terminals 902, 903 (also marked A, B) are both located on the same side (this for example can be the device's proximal end).

In a general for such a sensor, each of the N discrete coils exerts a magnetization force Hᵢ(I)=kᵢI on the surrounded magnetic core segment, which depends on the electrical current I flowing through the coil (which is identical for all coils). kᵢ is a constant factor which depends on the geometry of the coil, and in particular is in inverse proportion to the winding pitch of the coil (the denser the coil, the smaller the pitch, and the higher kᵢ). Denoted by Bᵢ is the component of the external magnetic field at the position of the i^{th} coil oriented in the direction of coil winding. The total magnetization force which is exerted on the i^{th} core segment is therefore:

$${H}_{i}^{tot} ( I , {B}_{i} ) = {H}_{i} ( I ) + {B}_{i} = {k}_{i} I + {B}_{i}$$

Accordingly, the total magnetization force Hᵢ^{tot} depends both on the electrical bias current I and external magnetic field Bᵢ. The inductance of the i-th magnetic coil can be computed using the magnetization-to-inductance relationship, given by the pre-calibrated curve μ(H) (of which an example is shown in FIG. 8):

$${μ}_{i} ( I , {B}_{i} ) = {l}_{i} \cdot μ ( {k}_{i} I + {B}_{i} )$$

Where lᵢ is the length of the i-th coil element (which is known in advance or found in pre-calibration). Finally, the total measured inductance of the complete coil (comprising individual coil elements, connected in series), for a given bias current I and given external magnetic fields Bᵢ is:

$${μ}_{tot} ( I ) = {μ}_{tot} ( I , {B}_{1} , {B}_{2} , … , {B}_{N} ) = {\sum }_{i = 1}^{N} ( {l}_{i} \cdot μ ( {k}_{i} I + {B}_{i} ) )$$

Over a given short period in time (e.g., a few hundred microseconds, for example, 100-1000 μsec), {Bᵢ} can be treated as constant for purposes of these measurements; at least, so long as the transmitted magnetic fields are relatively low in frequency, e.g., <200 Hz. The various values of {Bᵢ} are unknown and need to be solved from the integrated inductance sensor data. They cannot be solved from a single measurement and μₜₒₜ's equation alone. However, I is completely controlled by the sensor, so that the sensor can measure the total inductance μₜₒₜ(I) of the complete coil under M different values of I. For example, 16 different current values are used in measurements, distributed (e.g., uniformly distributed) between −100 mA and 100 mA. In some embodiments, within the same range or a different range of currents, at least 8 current values are used, at least 12, at least 16, at least 20, or another number.

The measurements with different currents yield a system of M non-linear equations, due to the non-linearity of μₜₒₜ(I):

$${\sum }_{i = 1}^{N} ( {l}_{i} \cdot μ ( {k}_{i} {I}_{1} + {B}_{i} ) ) = {μ}_{tot} ( {I}_{1} )$$

$${\sum }_{i = 1}^{N} ( {l}_{i} \cdot μ ( {k}_{i} {I}_{2} + {B}_{i} ) ) = {μ}_{tot} ( {I}_{2} )$$

$$⋮$$

$${\sum }_{i = 1}^{N} ( {l}_{i} \cdot μ ( {k}_{i} {I}_{M} + {B}_{i} ) ) = {μ}_{tot} ( {I}_{M} )$$

This system can be solved, for example, using non-linear optimization approaches (e.g., Gradient-Descent, Levenberg-Marquardt). To ensure that the system is solvable and has a unique solution, {lᵢ}, {kᵢ} are preferably chosen to maximize differences between the equations. For example, in the degenerate case where all {lᵢ} and {kᵢ} are equal, it is clear that the system is unsolvable due to the symmetry of {Bᵢ}, as they can be swapped without affecting the total measured inductances.

Adjusting {lᵢ}, {kᵢ} appropriately helps ensure the system is solvable. This is achieved, for example, by wrapping the coils with sufficiently varying pitches, e.g., as depicted in FIG. 9. For example, the coils can be wrapped with an exponentially increasing pitch. For example, in the case of 5 discrete coils, the pitches may be: 0.01 mm, 0.02 mm, 0.04 mm, 0.08 mm, and 0.16 mm. Alternatively, all the coil elements may be wrapped with minimal pitch (dense wrapping), but each coil can contain a different number of layers. A conductive wire (for example, 44 AWG enameled gold wire) can be wrapped from left to right, creating the first layer of the coil elements, then wrapped back from right to left. It can then be wrapped again from left to right, stopping before the most distal coil, then back to left. It can then be wrapped again from left to right, stopping before the 2^{nd} most distal coil, then back to left and so on. In such manner the coils will contain 2, 4, 6, 8, 10 . . . layers which will be reflected as kᵢ=2k₀, 4k₀, 6k₀, 8k₀, 10k₀, . . . in the magnetization forces Hᵢ.

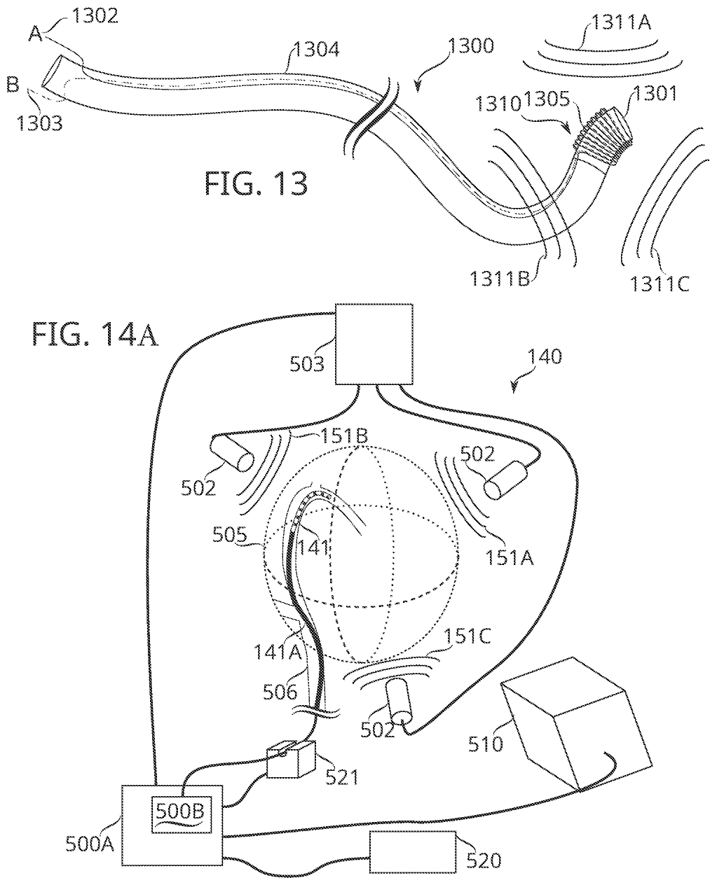

Full 3-D spatial localization (optionally with additional degrees of freedom specifying local orientation, too) may be provided when each location in space can be identified by a unique electromagnetic signature, or at least, electromagnetic signatures which are distributed so as to allow only one plausible compatible configuration (location and shape) of the curve sensor. With a single magnetic field (which can be variable or constant, e.g., as produced by a DC current), this is generally impractical, unless the shape and position of the curve sensor are known to be confined in some other way, e.g., confined to a single plane. In that case, the fixed length of the probe may effectively constrain what solutions can account for variations along its length in the single magnetic field's strength. Constraints of this and other types can also be used to assist 3-D spatial localization, as described in relation to FIG. 13.

For more general case, a plurality of magnetic fields may be provided, for example as described in relation to FIG. 13. Magnetic fields crossing through a region but oriented in different directions can “tag” it with a plurality of coordinate values. For example, three magnetic fields which are roughly mutually orthogonal in a region can set up coordinates arranged like the coordinate axes of a 3-D Cartesian space. The magnetic fields are not necessarily orthogonal, however, nor do they necessarily even vary along straight lines. More fields than three may be provided, e.g., to help ensure that positions in all regions of interest can be sufficiently identified and distinguished from one another, e.g., distinguished with a resolution suited to navigation needs. Strengths of the magnetic fields may be known, for example, from a model modelling the magnetic fields set up by a particular operating configuration of transducers. The model may be generated by any suitable combination of measurements, calibration assumptions, and/or theoretical calculations, as is known in the art.

A flexible approach to combining the magnetic fields in a single location while allowing them to be distinguishable varies each field at a different frequency. Spectral decomposition can then isolate their various influence. The frequencies used can be fairly low, e.g., less than 500 Hz. Additionally or alternatively, the magnetic fields can be combined by temporal multiplexing, e.g., by alternating their activation fully between on and off.

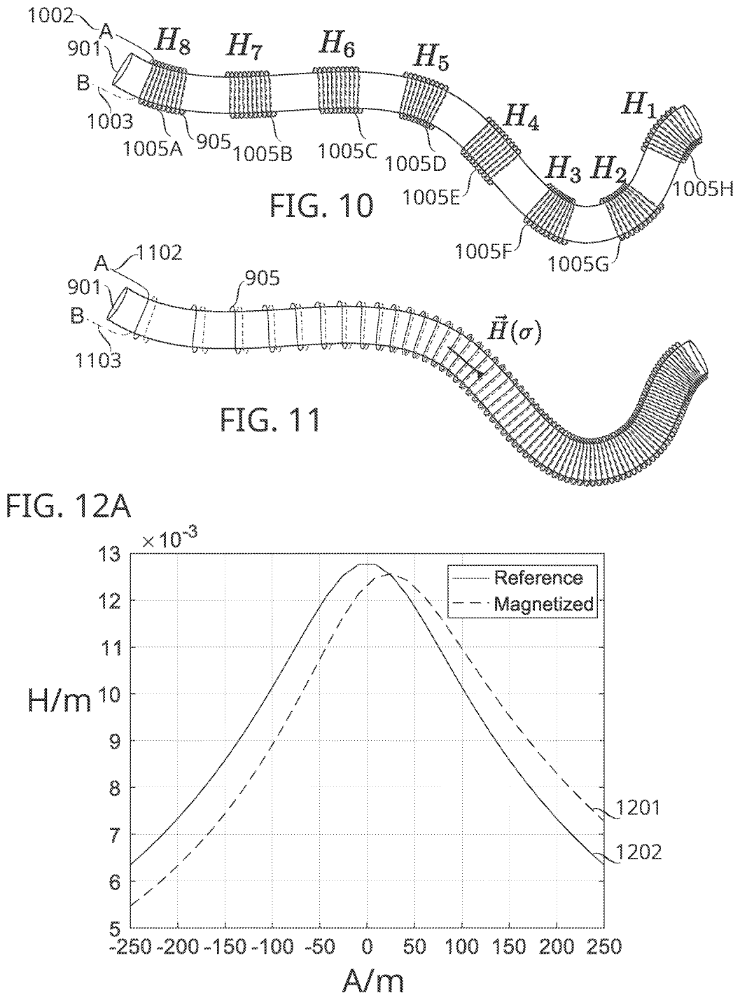

Reference is now made to FIG. 10, which schematically represents a flexible sensor comprising a single coil made of 8 discrete coil elements connected in series, according to some embodiments of the present disclosure.

In this case, each coil element 1005A-1005H (associated with a magnetization force H₁-H₈) is magnetized by a magnetization force Hᵢ which is determined by the external magnetic field Bᵢ and the coil configuration which affects kᵢ and lᵢ (number of windings, winding pitch, number of layers etc.).

To construct such a configuration, wire 905 may be wrapped in sections corresponding to each coil, and the sections joined after winding. Inter-coil connections are not shown. To facilitate joining the coils, there may be a pair of independent windings in each of coil elements H₁-H₈, with one lead on each side of each coil leading distally from lead end 1002 (also labeled A), and a second lead on each side of each coil leading proximally toward lead end 1003 (also labeled B). Optionally, each coil has two ends, with wire 905 extending straight, e.g., from lead end 1003 to the distal (right-side) end of core 901 to attach to the distal end of the last winding (H₁) there.

Reference is now made to FIG. 11, which schematically represents a flexible sensor comprising a coil with continuously decreasing winding pitch, according to some embodiments of the present disclosure.

In the example in FIG. 11, the winding pitch decreases monotonically (the winding density increases accordingly) until the coil reaches the distal part of the sensor. The rate of pitch change itself can be constant or varying. Winding of wire 905 may be performed, for example, from lead end 1102 (labeled A) to the distal end with decreasing pitch, and then back again to lead end 1103 (labeled B) with increasing pitch. Alternatively, the pitch may, for example, increase towards the sensor's tip, or the pitch may vary non-monotonically, or non-continuously or in any other similar way.

Lacking well-defined inflections in winding or spacing to mark distinct units (as, e.g., shown in FIGS. 9-10), the magnetization force per unit length in this case does not change step-wise. To analyze this, the magnetization force applied on the flexible core at each length parameter σ∈[0,L] along its curve (where L is the complete coil's total length) can be given by a continuous function H(σ). Similarly, the winding pitch can be given by P(σ) (where σ is same as before).

The continuous winding pitch P(σ) can optionally be modeled as a spline interpolation between a discrete set Pᵢ, corresponding to length parameters σᵢ∈[0,L]. The number of points in the discrete set can be selected according to what is needed to fo!r!0 coil; e.g., more points used for less-smooth or more direction-changing pitch variation patterns.

Assuming changes in the external magnetic field along the sensor's curve B(σ) are also well represented by a spline curve set of order N (usually the case), both the magnetization force and the external magnetic field can also be modeled as a spline interpolation between discrete points, Hᵢ, Bᵢ at σᵢ. If necessarily, the size of the set of spline points can be increased according to requirements imposed by irregularities of the magnetic field variations.

With this transformation adopted, the problem of measuring the continuous external magnetic field along the sensor curve of the continuous sensor coil in FIG. 11 reduces to the problem of finding a set of discrete magnetic fields Bᵢ at points σᵢ along the sensor's curve, which can be solved, e.g., as explained for in relation to FIGS. 9-10.

It should be understood that there are other ways of providing inductance which is variable along a longitudinally extended axis of a probe, alternatively or additionally to the use of one or more of those described in relation to FIGS. 9-11. Apart from variations in coil winding and/or coil composition, the core can be varied in composition and/or dimensions. For example, in some embodiments, the core diameter varies along the sensor's length. In some embodiments, the magnetic core's alloy composition varies along the sensor's length.

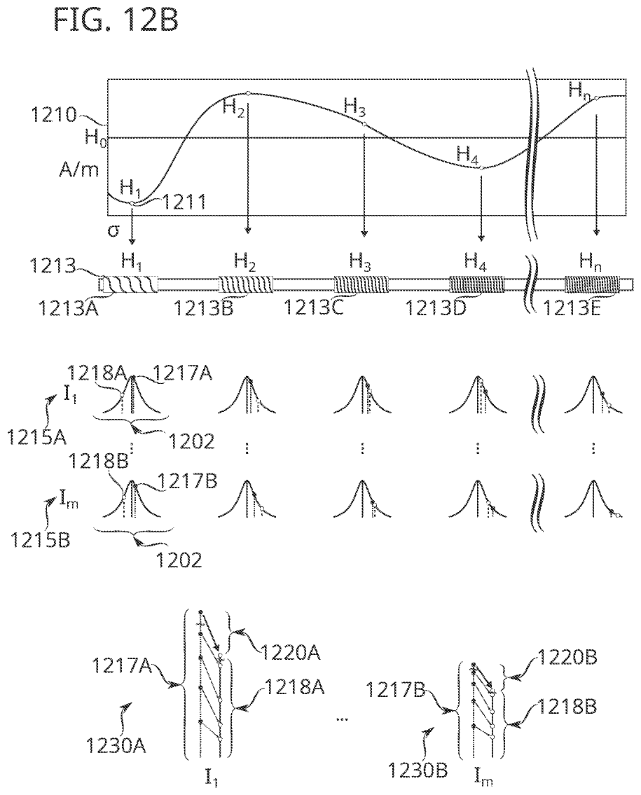

Reference is now made to FIG. 12A, which plots a magnetization to inductance relationship of an example magneto-inductive coil with varying pitch, under an externally applied magnetic field which varies along the sensor's curve, according to some embodiments of the present disclosure. Reference is also made to FIG. 12B, which schematically indicates features of data acquired using a magneto-inductive sensor 1213, according to some embodiments of the present disclosure.

In the example of FIG. 12A magnetization (the horizontal axis in A/m) is related to permeability (“inductance per meter”, vertical axis in H/m) relationship calculated for a flexible curve inductive sensor made of 4 discrete sub-coils connected in series, wrapped around a high permeability non-linear flexible magnetic core, each discrete coil having a different winding pitch.

The coils can be formed, for example, by winding a single long conductive wire with varying winding pitches in 4 discrete steps. A measurement circuit electronically samples current and/or voltage through the coils assembly using just 2 terminals which are the endpoints of the long wrapped wire forming the coils.

Curve 1202 shows the reference condition for the probe without an externally applied magnetic field. The probe bias may be set to any suitable part of this curve, for example, 150 A/m.

The example sensor is then positioned inside a spatially varying magnetic field such that each of the discrete coils “sees” a different magnetic field.

In the example of FIG. 12B, graph 1210 shows various magnetic fields H₁ through Hₙ, each corresponding in its position to one of coil regions 1213A-1213E. of magneto-inductive sensor 1213. Position in graph 1210 is shown along parametric axis σ, which is parameterized by distance along magneto-inductive sensor 1213. Each offset along σ also corresponds to a position in space, e.g., a position characterized by positions

$$r ( σ ) = ( \begin{matrix}σ \\ σ \\ σ\end{matrix} )$$

along the transmitter's orthogonal coordinate axes, or otherwise characterized.

The target intermediate result is measurement of the plurality of different magnetic field strengths along the length of the probe (in the case of FIG. 12A, N=4 fields; in the case of FIG. 12B, there are n fields H₁ . . . Hₙ indicated in graph 1210).

This intermediate result can then be referred to known or predicted magnetic field strengths at different locations in space in order to determine where the probe is, and/or what its present shape is.

With reference to FIG. 12A: to obtain data for calculating the target intermediate result, measurements of inductance μₜₒₜ(I) of the complete coil under M different values of I are obtained. Curve 1201 shows a different relationship between inductance and magnetization which is calculated to be consistent with what has actually been measured. The specifics of the changes from the position of curve 1202 are due to the combined effects on total inductance due to the specific values of the external magnetic field experienced individually by the four coils.

In FIG. 12B, elements of this process are further broken down as follows. Throughout a measurement period, the field strengths H₁ . . . Hₙ impinge upon coil regions 1213A-1213E, respectively, as noted by arrows leading between them. Rows 1215A, 1215B represent a model context for measurements of inductance per meter made at each current in a range of currents I₁ . . . Iₘ.

Each instance of the bell-shaped curve 1202 repeated in each row represents a same inductance curve with horizontal units of magnetic field strength A/m, for example as shown in FIG. 12A.

The positions of each of the black dots 1217A relative to its respective inductance curve 1202 represent (along the X-axis) magnetization away from the center peak induced by current I₁. Along the Y-axis is shown the expected corresponding inductance per meter for current I₁ in the absence of an external magnetic field e.g., if external magnetic field strength fell along the line marked H₀ in graph 1210, instead of the curve marked H₁ . . . Hₙ. The X-axis offset is different for each column position, since the different constructions of impedance regions 1213A-1213E result in different partial impedances for the same current. As a result, there is also a different Y-axis position for each black dot 1217A.

The black dots 1217B represent the same, but for a different current Iₘ. Generally, if Iₘ>I₁, then the X-axis offset of each black dot 1217B from the peak of graph 1202 centered on 0 A/m is also larger than the corresponding black dot 1217A in the same column. Ratios of offsets along the X-axis at different currents are not expected to be in constant proportion to the current ratio, since the current-induced magnetizing fields are themselves susceptible to permeability non-linearities.

Under conditions where the magnetic field strengths H₁ . . . Hₙ are imposed, inductances for each current I₁ . . . Iₘ at each position 1213A-1213E shift once again to reflect the combined magnetic field vector. In FIG. 12B, this shift is represented by hollow dots 1218A, 1218B. Along the X-axis, the shift is expected to be about the same for any given position 1213A-1213E under all measurement currents used, since the external magnetic field is not itself dependent on local permeability. However, the magnitude of shift in local permeability of the sensor along the Y-axis is different for different currents, since the starting offset is different, and different regions of the permeability curve have different slopes.

Although represented with separate values in each of rows 1215A, 1215B, the permeabilities (μ in H/m) of individual regions 1213A-1213E are not separately available in raw measurements. Instead, raw measurements sum all the inductances they contribute to for each measurement current I. The summed values correspond, e.g., to the vertical stack of inductance values corresponding to hollow dots 1218A (bottom left panel 1230A, for current I₁), and hollow dots 1218B (bottom right panel 1230B, for current Iₘ). Summed value is represented as height in these stacks. In panel 1230A, for example, the height of the stack of values 1217A represents

$${\sum }_{i = 1}^{N} {μ}_{i} ( {I}_{1} , {H}_{0} ) ,$$

and the height of the stack of values 1218A represents

$${\sum }_{i = 1}^{N} {μ}_{i} ( {I}_{1} , {H}_{i} ) .$$

The sum is optionally weighted by element length (in effect converting permeability to inductance proper) if lengths of elements 1213A-1213E are not all the same.

The differences 1220A, 1220B between stack heights for a particular current represent a total external magnetic field strength effect on inductance compared to baseline. These stack heights correspond also to points on curve 1201 of FIG. 12A—they are the “shifted” inductances.

To extract individual permeabilities (inductance if considered as length-weighted) from these measurements, the various summed heights are mathematically “explained” through the model's inductance curve, in a way that consistently proposes the same underlying values of H₁ . . . Hₙ no matter what current I₁ . . . Iₘ was used to produce the measurement.

As described, e.g., in relation to FIG. 9, the various inductive elements 1213A-1213E are preferably configured so that measured total inductances through a range of currents I₁ . . . Iₘ can only be “explained” in the context of the other known parameters of the situation for particular values of externally applied magnetic field strengths H₁ . . . Hₙ along the sensor's curve. The solution may be unique, so long as the design of the probe and measuring conditions was selected to avoid mathematical degeneracy in the solution process.

Next, the curve 1202 (that is, a pre-calibrated inductance to magnetization curve of the sensor) can be used to search for the magnetic field values which yield the measured inductance values. In this way, the plurality of magnetic field values along the sensor can be solved (in this example, N=4 values).

To proceed from this stage to the calculation of sensor position and/or curve, knowledge of how external magnetic fields area actually distributed in space is used. In some embodiments, shape and/or position sensing also comprises determining magnetic fields for a plurality of separately generated magnetic fields, so that locations in space can be individually identified according to the strengths and/or directions of the different magnetic fields which overlap within them. In some embodiments, one or more additional constraints are used: for example, the fields are constrained to be smoothly varying, and/or the shape and/or positions are constrained to be physically plausible shapes and/or positions of the probe.

### Endoluminal Device Integration

Reference is now made to FIG. 13, which schematically represents a guidewire 1300 having an integrated flexible sensor 1310 comprising a coil 1305 wound around a core 1301 which is constructed as a distal extension of the main guidewire body 1304, according to some embodiments of the present disclosure.

In some embodiments of the present disclosure, the physical principles described in relation, e.g., to FIGS. 9-11 for a flexible curve inductive sensor are used to provide a body-integrated EM sensor 1310 to a device 1300. Sensor 1310 may be provided in such a way as to replace a portion of the body of the device 1300, e.g., to replace a section of the length of the device 1300. In some embodiments, the device 1300 is a guidewire; optionally, the device 1300 is another longitudinally extended device incorporating a wire portion, for example, a catheter comprising at least one section of helically wound wire wall.

Additionally or alternatively, replacement of a portion of the host device body with the body of a sensor probe is optionally applied to a probe providing full curve sensing. In some embodiments of the present disclosure a shape sensed probe (elongated device with variable inductance density along its length) is constructed which is fully curve-tracked in 3-D in real-time relative to an EM transmitter (e.g., as further described hereinbelow, first with respect to device 1300 and then generalized to full curve sensing). The shape sensor may constitute a 40-44 AWG or thinner (e.g., 44-54 AWG) copper or gold wire wrapped around a high permeability non-linear flexible magnetic core, such as a supermalloy wire (for example, a 0.1 mm-0.3 mm core wire). The coil may be wrapped in varying pitch, and/or the core diameter may be varying along the sensor's curve to provide informatively sensed inductance curves which are indicative of the sensor's full shape in space relative to the transmitter. The sensor's coil is optionally used together with the core to provide (and potentially improve) mechanical properties associated with the probe portion of the host device, such as: steerability, torqueability, and/or pushability of the probe. The sensor's full length may be tracked (for example, 40 mm-200 mm of its distal part) or the sensor's distal end, or just the sensor's tip (in which case, it may reduce to an embodiment similar to that of FIG. 13).

### 3-D Spatial Localization of Shape and Position

### Basic Multi-Field Spatial Position Localization

With continuing reference to FIG. 13: in some embodiments, an EM transmitter is used, generating known EM fields of known frequencies and amplitudes, for example, low-frequency fields, for example 3-6 fields 1311A, 1311B, 1311C of different geometries at distinct frequencies below 500 Hz. The fields intersect to “tag” positions within a region, allowing each position to be distinguished according to the strengths of the various magnetic fields that pass through it, and/or their orientation.

In the example of FIG. 13, a single, optionally uniform coil 1301 is wrapped around a high permeability non-linear magnetic core 1301 at the tip of device 1300. This is then used as a sensor (e.g., connected to suitable sensing electronics via connections 1302, 1303) that measures the magnetic field at the sensor's tip. Since the transmitted fields 1311A-1311C are rather slow compared to the frequency at which sensor inductance can be measured (for example, the fields may vary with frequencies smaller than 500 Hz) the sensed fields can be measured with more than sufficient sampling rate to allow their decomposition into the different transmitted fields, for example using Discrete Fourier Transform (DFT) of the time-series or using correlation methods or using any other suitable method.

The sensed fields can then be used to solve the position and orientation of the sensor relative to the EM transmitter. With a single, spatially uniform coil 1305, the sensor 1310 may only sense the projection of the transmitter EM fields along its proximal-to-distal axis, which can provide a 5-DOF (5-Degrees of Freedom) solution of the sensor's tip in space relative to the transmitter. This amounts to 3-DOF position and 2-DOF orientation, where the roll angle of the sensor 1310 is missing.

### System for Multi-Field Spatial Localization and Shape Sensing

Reference is now made to FIG. 14A, which schematically represents an endovascular tracking system 140, according to some embodiments of the present disclosure.

Sensor 141 is a multiplexed, thin and long (longitudinally extended) variable inductance sensor, corresponding, for example, to one or more of the embodiments of FIGS. 9-11, FIG. 13, or another sensor using principles of variable inductance (in response to magnetic fields, e.g., FIG. 13) and/or with varying inductance arranged along its length (e.g., FIGS. 9-11). Additionally, systems according with descriptions of FIG. 14A (and optionally used according to descriptions of FIG. 14B) optionally include features described in relation to FIG. 5; for example in combinations also described in relation to FIG. 5.

In the example shown, sensor 141 is interconnected with external processing unit 500A and in particular readout controller 500B (e.g., via its connection terminals, for example, two connection terminals). Through the connection terminals, readout controller 500B drives sensor 141 with electrical currents used in magnetic field sensing. Moreover, readout controller 500B senses the magnetic field-reactive state of sensor 141, via the influence of the (variable) inductance of sensor 141 on properties of the electrical currents. The properties may include, for example, oscillation frequency of an oscillating circuit including the inductance of sensor 141, and/or amplitude of oscillation of such a circuit at one or more test frequencies. In particular, the properties are measured for a plurality of conditions distinguished by different driving current values.

Further operations attributed to the functioning of readout controller 500B optionally include processing of the current property measurements to indicate the state of a sensing region of sensor 141. The state is indicated as measurements, these measurements being indicative of local magnetic field (e.g., its strength) for a spatially distinguishable (although potentially in part overlapping) plurality of locations along the sensing region.

More particularly, influences on a current's properties indicate the total inductance (while subjected to that current) of sensor 141. The indications of total inductance are also referred to herein as “inductance information”, and this information is considered to be encoded in electrical signals which result from readout controller 500B driving current through sensor 141. Considered together with the driving currents used and other data about the construction of sensor 141, the inductance information encoded in these electrical signals can be transformed into measurements of local magnetic field for a plurality of locations along the sensing region. The locations are also distinguishable by their inductance density, or inductance they provide per unit of length. The difference in inductance density helps support the conversion of current property measurements to the measurements indicative of local magnetic field.

Still more particularly: the total inductance is in turn reactive to the driving currents, since in flowing through the inductive elements of sensor 141 those currents induce magnetization that temporarily affects the electromagnetic permeability of the longitudinally extended, high-permeability, and non-linear inductive core material of sensor 141. Since, furthermore, inductance density varies along the length of the sensing region, so does magnetization. This variation in permeability (or linear “inductance density”) is also referred to herein as inhomogeneous and/or non-uniform inductance density/permeability. In a first-order sense, this comprises baseline non-uniformity; that is, a non-uniformity independent of the non-linear changes in permeability next described.

The non-linearity of permeability change response as a function of magnetization means that different currents produce different relative amounts of inductance change in the plurality of locations along the sensing region.

After accounting for expected inductance changes resulting from self-generated magnetic fields, there may be residual deviations of the total inductance from, e.g., the zero-field inductance. The residual deviations from the reference are attributable to further influences on electromagnetic permeability, caused by magnetic fields generated externally to sensor 141 but also intersecting magnetic field sensor 141. Though initially measured as part of a total inductance, the residual deviations are indicative of integrated effects on individual parts of the inductance of sensor 141. Specifically, the inductance in each spatially distinguishable sensor location changes according to the magnetic field the sensor location interacts with.

Since, again, permeability changes are non-linear according to biases imposed by the particular current used to measure inductance, the individual changes are larger or smaller for the same local external magnetic field under conditions of different currents used in measuring inductance.

Informally, for a particular prove location, the particular set of magnetizing field strengths generated by a set of currents produces a “fingerprint”, defined, for example, as a function of change in permeability plotted against the measurement current. For a design of sensor 141 with a suitably inhomogeneous (non-uniform) baseline permeability, locations in the sensing region can be distinguished by this fingerprint. As one result of this: with different measurement currents applied, the distinguishable locations contribute respectively to changes from a baseline inductance in different relative amounts.

In some embodiments, the distinguishability of those sets of fingerprints is characteristic of what makes the locations distinguishable. For example, if two arbitrary regions are configured with inductance densities such that they share exactly the same set of fingerprints, they will sufficiently co-vary with changes in measurement current (even if exposed to different field strengths otherwise), as to become conflated in the measurement data as a single region.

Insofar as the construction of sensor 141 (i.e., with sufficiently distinct inductance densities) avoids creating this condition, the lumped inductance effects thus become potentially susceptible to mathematical separation,

Accordingly—using further inputs comprising, e.g., suitable calibration data and knowledge of how sensor 141 is constructed—mathematical processing performed, in some embodiments, by readout controller 500B, transforms the measurements of total inductance for different currents into measurements of local magnetic field for a plurality of locations along the sensing region of sensor 141. The processing may be carried out, for example, as described in relation to FIGS. 8-13 and in optionally in particular as described in relation to FIG. 9, or in an equivalent fashion.

Parenthetical to the hypothetical case of fully co-varying regions example given above: in some embodiments, degeneracy due to co-variance of inductance responses to magnetic fields for different regions (insofar as it is present, perhaps partially) is at least partially overcome. To begin with, even if the two regions statically experience different magnetic field strengths that remain mixed in a mathematical separation, the mixed value may vary with current in a way inconsistent with any single field strength. That, in turn, may allow deduction of separate magnetic field strengths which are consistent with the mixed value; though not, in the pure case, assignment of these to one or the other particular region.

Other information may allow this assignment, and/or be useful in determining the separate magnetic field strengths. For example, continuity constraints can be derived from knowing that the sensor comprises a continuous linear shape within continuously (over distance) varying fields. If, e.g., the above-mentioned “arbitrary regions” with the same inductance densities are also physically separated by other, distinguishable regions, then a solution may be estimated which jointly preserves a metric of continuity with neighboring regions, motivating a division of the jointly measured magnetic field strength into unequal contributions that “pay” for reducing discontinuity. In some embodiments, this approach is used to potentially create and/or increase mathematical separability of different locations along the sensing region. For example, applying the continuity constraint, a larger number of distinguishable regions may be separable within a given range of inductance densities among which those regions are distributed. In some embodiments, inductance densities of separable locations are arranged along the longitudinal extent of a sensor 141 such that differences with neighboring locations are maximized, and/or such that physical distances from the most similar locations are maximized. Together with this, constraints on continuity are applied. Error penalties for violating continuity constraints are reduced by re-balancing the relative assignment of field strengths for more distant pairs of locations with more similar inductance densities.

Optionally, at least portions of the system elements which perform functions of readout controller 500B are provided in an enclosure separate from external processing unit 500A. These portions of readout controller 500B then communicate with the processing unit 500A, at least to convey data in some form from sensor 141. Insofar as further processing is performed after this conveyance to perform the transformation into local magnetic field measurements, processing unit 500A also acts as a portion of readout controller 500B.

Optionally, portions of readout controller 500B responsible receive commands from processing unit 500A governing control of readout to suitably drive and directly measure from sensor 141. Additionally or alternatively, the portions of readout controller 500B responsible for driving and direct measurement are separately (e.g., manually) configurable.

External processing unit 500A may incorporate further processing features; for example as described in relation to FIG. 5, 14B, or other embodiments herein.

Also provided, in some embodiments, are EM field control unit 503, interconnected with a plurality of field generators 502 (e.g., magnetic field generators). The field generators 502 generate distinguishable magnetic fields 151A-151C; for example, distinguishable according to a frequency of their generation. Field generators 502 are arranged around navigational region of interest 505, comprising, for example, a portion of a human body (e.g., a cranium). The local strengths and/or directions of fields 151A-151C are at least partially uncorrelated with each other, so that field measurements made within a region of suitable size to overcome signal-to-noise limitations are distinguishing of that region from adjoining regions, preferably in any direction.

In some embodiments, sensor 141 is part of (e.g., attached to or integrated within) another device, for example, endoluminal device 141A, which comprises a long, thin body sized, shaped, flexible, pushable, and otherwise configured for endoluminal navigation that advances a tip of endoluminal device 141A through narrow lumens of the body. Endoluminal device 141A optionally has a length to diameter ratio greater than 100, and typically a much larger such ratio; e.g., in the range of several hundred to about a thousand (e.g., 1 meter long, 1 mm in diameter). Sensor 141 may be a body-integrated sensor of endoluminal device 141A, in the sense described for the single-coil embodiment of FIG. 13.

Sensor 141, as part of a navigable endoluminal device 141A, is shown introduced into navigational region of interest 505, via a vasculature 506, of which a restricted portion is shown for purposes of illustration. Endoluminal device 141A may be, for example, a guidewire, catheter, or other long and thing (longitudinally extended) tool suitable for insertion into longitudinally navigated anatomical channels such as blood vessels and/or airways.

Among the potential advantages of sensor 141 is its optionally very small cross-section. Its inductive core (e.g., a wire of having variable permeability in response to magnetic fields) may be roughly equal to or less than the diameter which is provided anyway to non-sensing portions of endoluminal device 141A. It is wrapped with fine coil windings, preferably significantly thinner still (e.g., 40-54 AWG) which potentially increase this diameter by only a small fraction (e.g., less than about 40%, less than about 33%, less than about 25%, or less than about 15%). For example, 44 AWG wire is about 50 μm in diameter, so that it adds a total thickness of about 100 μm. The overall diameter may be, for example 1 mm or less, 750 μm or less, 500 μm or less, 400 μm or less, 350 μm or less, or 250 μm or less. The diameter may be different at different positions along the proximal-distal axis of the sensor 141, e.g., between diameters in a range between about 1.5 mm and 250 μm. For example, the inductive core may be tapered from about 900 μm on a proximal side and about 300 μm on a distal side, with the added coil windings adding approximately another 100 μm for a total width range between about 1000 μm and about 400 μm. The ratio between largest and smallest diameters along the sensing region of sensor 141 may be, for example, at least 1.5, 2, 2.5, or 3. The sensor may itself provide the mechanical properties which provide the mechanical navigability properties device 141A along its length, so that other elements alongside it are unnecessary. The variable permeability material may be provided alone as the core material along the extent of sensor 141, or it may be mixed in with other material, e.g., as part of one or more alloys, within a matrix (e.g., a polymer matrix), as strands of different compositions, or in another manner.

Another potential benefit of such sensors is design simplicity and cost, with corresponding potential advantages for use as a disposable device. For example, in certain applications a plurality of magneto-inductive fully tracked “guidewires” can be introduced to a patient's organ and provide a fully tracked real-time skeleton of that organ for certain uses, such as modeling the deformation of that organ during a medical procedure.

Within the context of a minimally invasive medical procedure relying on endoluminal (e.g., endovascular, or more particularly, neurovascular or cardiovascular) guidewire navigation, there may be more than one sensing modality in place. For example, external imaging device 510 is optionally provided, which is optionally an X-ray imager. Ionizing radiation-based imaging, while well-known and commonly used as a way of monitoring endovascular instrument positioning, has associated exposure risks. In some embodiments of the present disclosure, position determination using sensor 141 allows reduction of ionizing radiation exposure, e.g., reduced imaging frame rates (e.g., 5 Hz instead of 30 Hz, or another factor of frame rate reduction). In some embodiments, use of fluoroscopy is reduced to obtaining occasional recalibration and/or verification images, for example every few seconds or even minutes, with navigation proceeding in between guided by probe shape and position measurements. To maintain an acceptable level real-time feedback, interpolation of probe position through the “missing” frames optionally uses spectrally multiplexed position sensing to infer changes in position, e.g., from a previous X-ray “key frame” image. Alternative and/or additional processing operations using the mixed data stream may be used to provide feedback (e.g. displays shown using user interface 520) which help an operator of device 141A maintain an uninterrupted awareness of the results of their actions to navigate (e.g., steer, rotate, push, and/or pull) or otherwise operate device 141A, for example as described with respect to box 130 of FIG. 14B.

With respect to optional robotic driver 521 provided in some embodiments corresponding to FIG. 14A, features described in relation to robotic driver 521 of FIG. 5 also apply. In overview: in some embodiments, robotic driver 521 actuates movements of endoluminal device 141A for navigation as such. In particular, selection of these movements may be based on currently observed shape and/or position of sensor 141, and more particularly, based on the relationship of the current shape/position of sensor 141 to the relative location of targets such as vascular bifurcation branches. Additionally or alternatively, in some embodiments, movements may be actuated to help sense the present state and/or responsiveness to input of endoluminal device 141A. In particular, known small actuation inputs are optionally compared to observed corresponding changes in the shape and/or position of sensor 141. Optionally, larger movements are selected, suggested, and/or performed, based on these observed correspondences and the relative positioning of one or more navigation targets.

### Variations of the Shape/Position Sensor

With continuing reference to FIG. 14A, sensor 141 provides, in some embodiments, a flexible curve sensor comprising a single continuous coil wrapped around a flexible magnetic core, and usable to measure a plurality of magnetic fields at plurality of positions along its length, for example 4 or 8 or more fields and 4 or 8 or more positions. By sensing a plurality of magnetic fields, a system 140 incorporating sensor 141 may then solve (e.g., using processing unit 500A) for each of the plurality of positions a position and orientation of the sensor's curve relative to one or more EM transmitters 502. This can provide a full shape localization of the flexible sensor 141 relative to the EM transmitters 500A.

More particularly, in some embodiments, EM shape sensor 141 comprises a long continuous coil wrapped around a high permeability non-linear flexible magnetic core, for example, such as a supermalloy wire (also referred as a continuous EM shape sensor). The coil may have varying winding pitch and can comprise, for example, a 40-44 AWG copper or gold wire. The wire can be wrapped from left to right and then in a second layer from right to left such that the 2 terminals of the coil are available on the same proximal side of the sensor and are available for connection to the sampling electronics.

In some embodiments of the present disclosure, only the tip of sensor 141 is wrapped with a short coil, for example, a 3 mm-10 mm coil, to sense DC and/or time-oscillating magnetic field strength at the tip of the sensor and to optionally provide localization of the sensor's tip relative to an EM transmitter.

In other embodiments, only the distal end of sensor 141 is wrapped with a medium length coil, for example, a 10 mm-40 mm varying-pitch coil, (e.g. varying in pitch as depicted in FIG. 9), to sense a plurality of DC and/or time-oscillating magnetic fields at the distal end of the sensor 141 and to optionally provide data used by system 140 for curve localization (shape sensing) of the sensor 141 distal end relative to one or more EM transmitters 502. Sensor 141 may provide readings distinguishing, e.g., 2-4 positions along its coil-wrapped distal length.

In another embodiment, the distal part of sensor 141 is wrapped with a continuous coil which can optionally be of varying pitch, for example, a 40 mm-200 mm varying-pitch coil, for example, as depicted in FIG. 11, to sense a plurality of DC and/or time-oscillating magnetic fields at the distal part of the sensor and to optionally provide full curve localization (shape sensing) of the sensor's distal part relative to an EM transmitter. The sensor may provide readings distinguishing magnetic fields at e.g., 2-19 positions along its coil-wrapped distal part, which can be used to compute a plurality of positions and orientations along the sensor's curve to provide the sensor's curve position and orientation.

### Variants of A Shape Sensing System

With further reference to FIG. 14A, it should be understood that sensor 141 is optionally provided separately from an endoluminal device 141A, and may optionally be configured for operation as a separate device in its own right, or together with another device which is not an endoluminal device 141A (at least, not in the sense of being a medical endoluminal device). With reference to FIG. 14A as a baseline, the elements of a shape-sensing navigation system more generally may be understood to exclude medical endoluminal device 141A. Furthermore, external imaging device 510 is also optionally excluded, including in a medical endoluminal device implementation of system 140.

However, imaging device 510 may nevertheless be provided in some applications. Optionally it is provided other than as shown (i.e., within rather than outside the space that sensor 141 traverses). For example, imaging device 510 may be provided an optical imaging device which is physically interconnected with and moved along by navigation of sensor 141 itself.

For example, sensor 141 may be used to assist in manufacturing, inspection, and/or repair operations which involve the traversal of deep but confined spaces, e.g., using small access ports. In particularly, there is potential suitability of shape data for use in robotically guided navigation of such spaces, since the data may be directly produced in spatial coordinates. This potentially eliminates a need to directly overcome the numerous feature recognition issue which may arise in image-guided navigation approaches. In some embodiments, a largely image-guided navigation approach may be supplemented using shape sensing, e.g., to reduce computational complexity of traversing crowded and/or visually complicated environments or portions thereof.

Interconnection of processing unit 500A and user interface 520 is optional, at least in the general case. For example, a robotic navigation system may forgo a user interface which provides direct indications of shape sensing in normal use.

The EM field control unit 503 and field generators 502 are not necessarily provided together with a system 140, e.g., they may be separately provided as part of another magnetic sensing system, or sensing may be occurring in an environment which is magnetically “rich” for another reason, e.g., in the context of electrical power generation and/or transmission applications. In some applications, one or more permanent magnets may be sufficient to establish magnetic fields which determine at least device orientation, and potentially also shape in whole or in part, depending e.g., on how many magnets are used, and other constraints such as limitation of the sensor 141 to move in a restricted near-planar volume, near-cylindrical volume, or other restrictive and well-characterized volume shape.

These types of alterations should also be understood to apply, changed if and as necessary, to embodiments of systems using sensors such as are described in FIG. 5. In particular, references to magnetoresistive type sensors such as sensor 501 of FIG. 5 may substituted for references in this section to magneto-inductive type sensors such as sensor 141 of FIG. 14A, at least insofar as system configuration variations disclosed do not rely on or make particular reference to sensor characteristics which differ between the two sensor types.

### Spatial Localization Enhancements

In some embodiments, an EM transmitter (comprising control unit 503 and field generator 502 of FIG. 14A) used generates a small number of different EM fields (for example, 3-6 fields). FIG. 14A shows three fields 151A-151C; this number should be understood as a non-limiting example.

The field number can alternatively be large (for example, 20-30 fields). As the number of generated EM fields increases, the number of sensed fields increases accordingly and the conversions between the sensed fields to a position and orientation solution of the sensor may become more accurate and robust. However, it is sometimes necessary to keep the number of transmitted fields in the lower of these two regimes; for example, to reduce power consumption in the EM transmitter, reduce setup complexity, or another reason. With just 6 transmitted EM fields, each discrete magnetic field time-series along the sensor's curve can be converted individually to a position and orientation of that point relative to the transmitter. The system may remain at least somewhat exposed to noise and/or bias errors, however.

In some embodiments, shape, smoothness, and/or distance constraints derived from the construction of a sensor 141 (e.g., as a fixed-length, linear device) are used to potentially improve accuracy and/or robustness of the solution of the sensor's shape from measurements made using it.

For example, in some embodiments of the present disclosure: instead of solving the position and orientation of each discrete point along the sensor's curve individually, the complete shape of the sensor may be solved (e.g., by processing unit 500A) as a whole, under shape and smoothness constraints. The problem of solving a position and orientation (for example, 5-DOF) of points along the sensor's length can be viewed as an optimization problem. The goal of the optimization is to minimize the error between the sensed magnetic fields as a time series (or as signed amplitudes after a DFT conversion) and the known generated magnetic fields (the model). By finding the position and orientation of each discrete point along the sensor's length under the known generated magnetic fields which explain the measurements, the position and orientation of each discrete point is solved.

By imposing shape and smoothness constraints of the solved positions and orientations of the sensor's curve, the dimensionality of the optimization problem is reduced and the number of measurements can be reduced accordingly. For example, in FIG. 10 an EM shape sensor (continuous EM shape sensor) is depicted which comprises 8 discrete sub-coils connected in series. Using the methods described above, the sensor can convert the sensed inductive curve into 8 separate magnetic field measurements, corresponding to the 8 discrete coils. Instead of then solving 8 individual positions and orientations, the complete sensor curve may be solved under the known transmitted EM fields and by imposing shape and smoothness constraints, such that the magnetic field measurements are explained. For example, the pre-calibrated length between the discrete coils may be used to force the solver to solve 8 discrete positions along the curve which conform to the known lengths between the discrete coils. Additionally, the solver implemented by processing unit 500A may “punish” un-smooth position and orientation solutions between neighboring coils. This may allow the sensor to be operated under a small number of transmitted EM fields, for example, using just 3 fields, which may be insufficient when positions and orientations are solved individually, but can be sufficient once the sensor's shape is solved as a whole.

The terms “continuous EM shape sensor”, “flexible EM shape sensor” and “EM shape sensor”, “curve inductive shape sensor”, “curve resistive shape sensor”, “flexible inductive shape sensor”, “flexible resistive shape sensor” all refer to a continuous EM shape sensor.

In some embodiments of the present disclosure, the solution approach implemented by processing unit 500A is further generalized in the following manner: instead of decomposing the sensed inductance curve by the sensor to individual magnetic field measurements (for example, 8 different measurements), the sensor may solve the full sensor curve in space relative to the transmitter, under shape and smoothness constraints, such that the predicted inductance curve (according to a pre-calibrated model) of the predicted sensor's full curve position and orientation in space would yield the measured inductance curve. This may further reduce the dimensionality of the optimization problem and correct for potential errors in the conversion between the measured inductance curve and individually sensed magnetic fields. By imposing constraints (such as shape and smoothness constraints) and under the assumption that the sensor resides within some curved location at the proximity of a known and calibrated EM transmitter, only certain inductance curves are possible (over time) which are indicative under a constrained curved shape of the continuous EM shape sensor in space.

### Spatial Localization Enhancements to Calibration

In some embodiments of the present disclosure, the exact inductance to magnetic field relationship of a sensor 141 (FIG. 14A) may not be known in advance, and may need to be calibrated. Furthermore, the exact relationship between some curved position and orientation of the flexible sensor in space relative to an EM transmitter and the sensed inductance curve (over time) may be unknown. Furthermore, the exact frequencies and amplitudes of the transmitted EM fields may be unknown in advance. To solve this, a calibration process may be used where the flexible sensor is positioned in a plurality of curved positions and orientation in space relative to the transmitter.

The raw sensor data may be collected over time; for example, the full inductance curves over time may be collected. A model is then used which can include a plurality of variables, such as for example: the frequencies and amplitudes of the transmitted EM fields, the curved positions and orientations of the flexible EM shape sensor 141 over time, the relationship between externally applied magnetic fields along the sensor's curve and the sensed inductance curve by the sensor etc. These variables can then be solved, for example, under imposed shape and smoothness constraints of the curved EM sensor. As long as the dimensionality of the measurements (that is, the inductance curves sensed by the sensor over time) exceeds the dimensionality of the variables, then the variables can be solved in a calibration process using an optimization process. This can provide a very accurate unsupervised calibration of the EM shape sensor. In a more supervised setting, the dimensionality of the variables can be reduced, for example, by placing the curved EM sensor in known curved positions and orientations relative to the EM transmitter, thus removing the curved positions and orientations from the list of unknown variables during calibration to make the optimization process more robust. The result of the calibration process is a prediction model which can provide predictions for the sensed inductance curves of the sensor for a given curved position and orientation of the EM shape sensor 141 relative to the transmitter(s) 502. This prediction model can then be used to solve the curved position and orientation of the sensor 141 in real-time, providing shape sensing of the sensor 141, e.g., as described above.

### Endoluminal Navigation

Reference is now made to FIG. 14B, which schematically diagrams operations of a system for tracking of angiogram deformation and probe position using curve inductive sensor, according to some embodiment of the present disclosure.

Operations of FIG. 14B are grouped by enclosing boxes into 3 phases of processing: creating a volume from an angiogram (box 110), deforming the volume based on a tracked catheter (box 120), and using the deformed volume and detected tip and shape to display a navigational view (box 130).

Operations of box 110 overall are optional, and provided as an example of how a 3-D luminal structure model may be constructed. The model in the example of FIG. 14B is a model of vasculature. However, models used in some embodiments of the present disclosure, and in particular models used in accordance with a broad understanding of the methods of FIG. 14B, may include models of any anatomical lumen structure. The models are optionally generated from any data appropriate to what is available for the lumen structure. For example, when generated form image data, the image data provide contrast and resolution sufficient to establish baseline knowledge of how spaces of the lumen structure are positioned and interconnected. The level of detail is sufficient, for example, to support localizing a long and thin probe to within particular passageways of the luminal structure, and/or guiding the navigation of that probe therewithin.

Within box 110, in some embodiments, angiograms 111 are provided (just as a non-limiting example). Angiograms 111 image one or more states of the luminal structure which is to be modeled. Preferably, they include sets of images from different directions of the luminal structure which provide views of a simultaneous or effectively simultaneous state of the luminal structure. “Effectively simultaneous” optionally includes states which follow each other so quickly that changes in the features shown (e.g., their shapes and positions) are negligible for purposes of their uses as described herein, and optionally includes states which are repeated (e.g., at the same phase of respiration and/or the cardiac cycle) with similarly negligible differences. Optionally, “effectively simultaneous” states include sets of a plurality of states which are non-negligibly different from each other, but can be nevertheless readily be brought into spatially agreement based on their similarities, e.g., after identification of reference features and morphing to bring those reference features into registration.

At block 112, in some embodiments, each angiogram 111 is processed to detect vessels, for example, using such methods as digital subtraction, or other detection methods, e.g., as known in the art. The operations of block 112 are not necessarily performed at the indicated point in the process; for example, vessel detection as such may be performed after calculations which generate a volumetric estimation of vascular locations using angiograms 111 and knowledge of how they were generated.

Optionally, at block 114, in some embodiments, multiple phases of the detected vessels are optionally combined to form a single data structure (e.g., an image, linked list of coordinates, or another data structure) comprising all vessels of interest in the field of view. By the term “multiple phases” it is indicated that the images are not necessarily simultaneous or effectively simultaneous as the term was described above. In particular, injected contrast agent may wash through an imaged vasculature progressively, so that no one image shows all the vasculature of interest. Combination of images may operate, for example, on the image data in grey-scale form (e.g., by performing successive registrations of images based on overlap in shared regions of contrast agent), and/or on vascular locations identified in the images in block 112, and merged in the form of linked lists of positions, or in another format. The two types of information may be combined in order to perform the combination of block 114.

At block 116, in some embodiments, a tomo-synthesis type algorithm is used to combine images representing a plurality of view angles into a common 3-D space. For example, 2-D projections through a plurality of directions are combined into a 3-D volumetric image reconstruction using iterative back-projection techniques. Optionally, the vascular detection of block 112 is performed on data, which has been mapped into this comment 3-D space.

At block 118, in some embodiments, vascular segments with their associated 3-D spatial extends are combined to create a 3-D model of the vessels. The 3-D model comprises, for example, descriptions of paths along which vascular centerlines extend, and descriptions of nodes at which paths join and/or bifurcate. Optionally descriptions of vascular cross-sections along the paths are also provided, for example, vascular cross-section expressed as one or more radii (e.g., two radii of an ellipse, or a single circular radius). The vascular cross-section may be constant within a segment (e.g., representing an average cross-section) or variable. Variation in cross-section may be represented with separate values for each closely separated step along the vessel (e.g., steps of about a pixel width in the original image representation), or at lower resolution, e.g., a function of few parameters, such as a spline which represents tapering, and optionally includes control points to represent other features such as sclerotic narrowing.

In some embodiments, the 3-D model of block 118 is associated with a deformation model, which characterizes how the 3-D model deforms to accommodate other constraints. For example, it may be allowed to deform relatively little in length between segment-connecting nodes (bifurcations), but be compliant in terms of accepting changes in curvatures that extend between such nodes. Compliance assigned by the deformation may depend on factors such as vascular radius, assumptions about the compliance of tissue in which the vasculature is embedded, or other factors. In some embodiments, the model represents compliance as allowable variations in angles associated with ends of segments terminating at nodes. Optionally, compliance values are assigned separately along segment locations.

Additionally or alternatively, the model of deformation is assigned otherwise. For example, volumetric areas considered to be elastically deformable in response to perturbation by outside constraints, which may include propagation of deformations to maintain continuity, such that a continuous deformation field is maintained. Segments deform locally according to the local vector of the deformation field within which they are embedded.

Additionally or alternatively, the model of deformation is assigned otherwise. For example, it can be based on a finite-element deformation model in which each tissue element is assigned a set of deformation features such as stiffness, thickness, scalability, pushability, etc. The segmented vessels can be assigned with “pipe-like” features (forcing a skeletal structure type deformation) while in-between tissue can possess different deformation properties, so that the deformation tracking engine, based on finite-element deformation simulation, accurately and physically simulates the deformation behavior of the anatomical structure, for example, based on real-time measurements of a curve inductive sensor, as described below.

When there are angiographic images obtained during a procedure, the model produced at block 118 may be updated and/or regenerated as new images become available (for example in real-time). This can be done, for example, by re-projecting the current 3-D model (which was created using previous angiographic images) according to new X-ray images as they are acquired during procedure. The 3-D model can be projected using a calibrated X-ray camera model, from an exact known 3-D position and orientation of a virtual camera, which matches the position and orientation of the true X-ray device source, such that the re-projected 3-D model images would match the new angiographic X-ray images. Aligning the re-projected 3-D model images with new angiographic X-ray images can also involve finding the optimal X-ray source position and orientation that would yield the best match between the re-projected images and the new angiographic images, based on image matching metrics (such as least squares, mutual entropy etc.). After matching the re-projected 3-D model to the new angiographic X-ray images, any mismatch between the matched images can be attributed to anatomical deformation which occurred in the real anatomy but is not reflected in the 3-D model. The 3-D model can then be updated, for example, by using a deformation model as described above, for example, a parametrized deformation model, for example, a skeletal parametrized deformation model, and a deformation can be searched such that the re-projected deformed 3-D model will match the new angiographic images. In this way, a deformation can be searched (according to an assumed deformation model) that explains new angiographic X-ray images, which provides a mean to track the anatomical deformation with each new acquired angiographic image.

In box 120, in some embodiments, a curve inductive sensor 121 is provided. This particular sensor type is given in FIG. 14B as an example. In some embodiments, another sensor supporting curved shape tracking is provided, for example, a sensor relying on a variably resistive sensor, or another type of curved shape sensing. Sensor 121 is physically associated along its length with an endoluminal device such as a catheter or guidewire; for example, bound to it, embedded within it, and/or comprising a portion of the structural body of the device, e.g., a section of a guidewire.

At block 122, in some embodiments, the physical association is made use of to refer tracking information for the sensor to the associated extent of the endoluminal device. In the example of FIG. 14B, the endoluminal device comprises a catheter.

At block 124, in some embodiments, the 3-D shape of the catheter is reconstructed, using the tracking data and/or sensor raw data. Block 123 separately specifies detection of the catheter shape location, which optionally is in part calculated as part of block 124, and optionally also involves mapping locations described within a shape-detection coordinate system to a frame of reference also having a known relationship to the vasculature being navigated, and particularly, a known relationship to the 3-D model of vasculature, e.g., as generated at block 118.

At block 126, in some embodiments, deformation of the 3-D model provided as an output of box 110 (and block 118) is calculated, based on constraints imposed by the reconstructed 3-D shape of the endoluminal device, and its location. The deformation calculated is mediated by the deformation model aspect of the 3-D model. Deformation applied is preferably sufficient to allow fully consistent matching of the deformed 3-D model and the 3-D shape and position of the endoluminal device. Especially (but not exclusively) where deformation of the 3-D model alone approaches or exceeds reasonable bounds (e.g., as set by the deformation model), interplay between the vascular model and the shape of the endoluminal device may be implemented to additionally “correct” the detected shape of the endoluminal device. Such correction may reflect inaccuracies in shape detection calibration, measurement noise, transient data errors, or other factors. Modification of device shape in the model may assist in maintaining a presentation appearance (e.g., in box 130) which avoids distracting fluctuations and/or physically unrealistic configurations.

According to some embodiments, the deformation of the 3-D model can also be tracked by a combination of updating the deformation model by using new angiographic X-ray images, as well as based on a device's tracked 3-D curve, as described above. For example, the new angiographic X-ray images should match the re-projected deformed 3-D model, but the deformed 3-D model should also conform to the device's tracked 3-D curve (e.g., such that the device's tracked 3-D curve is fully or partially contained inside vessels of the deformed 3-D model). Different weighting can be used to balance between the influence of new angiographic X-ray images and a device's tracked 3-D curve on the deformed 3-D model.

Additionally or alternatively, since the shape of the sensor may be tracked in real-time, its 3-D position may be re-projected on the 2-D X-ray images by means of registration between the electromagnetic transmitter coordinate system to the X-ray coordinate system. In one embodiment, radiopaque markers can be incorporated on or inside the EM transmitter and can be seen by X-ray images. These markers can then be used to register between the X-ray 2-D projections and the EM transmitter, that is, to localize the X-ray imager in EM coordinate space (there may only be a unique possible 6-DOF position and orientation of X-ray imager that yields the projection of EM markers as seen in the X-ray imager). In the case of a Printed Circuit Board (PCB) EM transmitter, for example, one which contains copper traces which form EM transmission coils, for example, a flat PCB EM transmitter, copper can be deliberately added to some of the transmitter's PCB layers to form radiopaque markers for EM to X-ray registration. These markers can be point-based markers, circular, squares, grid markers, uniquely shaped markers or any other kind of markers which are radiopaque. The PCB marker positions is well known relative to the EM transmission coils, since these markers are designed on the same PCB and PCB manufacturing process is highly accurate (for example, accurate within 6-mil). Since the marker positions are accurate and known relative to the EM transmission coils, their positions in EM coordinate system are known. When an X-ray image is taken by X-ray imager, the 2-D projections of the markers are seen in X-ray and are matched to their 3-D known positions (in EM transmitter coordinate system). The 6-DOF position and orientation of X-ray imager can then be solved in EM transmitter coordinate system, to provide EM to X-ray registration, for example using Structure-from-Motion (SfM) methods, non-linear optimization methods or any other feature registration methods. The camera calibration of X-ray imager may be known in advance (for example, its field-of-view, image origin etc.) or can be solved online as part of the registration.

In the case of a general EM transmitter, general radiopaque markers with known EM position (relative to the transmitter) can be used to register between the X-ray and the EM coordinates, similarly as described above.

In some embodiments, in the case where EM reference sensors attached to the patient (for example, to the patient's cranium) are used, these reference sensors can also be used as a radiopaque markers whose EM position and orientation are known in EM coordinates (since they are being tracked). The reference sensors can be segmented/detected in acquired X-ray images and the X-ray source position and orientation can be solved in EM coordinates based on the real-time tracked reference sensors' position and orientation (as tracked by the EM tracking system).

Using the EM to X-ray registration, the tracked 3-D shape of the sensor (in EM transmitter coordinates) can be re-projected to the 2-D X-ray images of imager. This can be used to indicate to the operator where the probe is in the X-ray image, which may not be clearly viewed in the X-ray image due to poor image quality, occluded, saturated and/or blurry. The re-projected shape of the sensor may be used for example to visually enhance the probe in the X-ray image. It can also be used to draw the curve on the X-ray image, in cases where the probe may not be radiopaque enough in the X-ray image. It may also be used to match between the re-projected curve of the probe (as transferred from the 3D shape tracked in EM coordinates to the X-ray image) and the original 2-D X-ray image of the probe. This matching may be used to correct the EM tracked shape of the sensor as described above. The EM to X-ray registration may be used to create augmented views during procedure. For example, the positions of anatomical features of a patient may be known in EM coordinates, for example, due to EM to anatomy registration methods. For example, a 3-D roadmap of a patient may be localized in 3-D EM space. Patient's blood vessels, lesions, airways, 3-D roadmap, 3-D angiogram or other features may be localized in 3-D EM space. These features can be transferred and overlaid on X-ray 2-D images using EM to X-ray registration methods as described above. The tracked shape of the sensor may also be overlaid or the sensor's curve can be enhanced on X-ray image. The operator may then be presented with a richer and/or enhanced and/or augmented X-ray image on which faster procedures may be performed (instead of performing procedures on raw, un-enhanced, un-augmented X-ray images, where important anatomical features may be hardly seen).

Additionally or alternatively, EM to X-ray registration may also be achieved without markers, or with partial markers, by matching the tracked 3-D shape of a sensor with a segmented 2-D shape of the sensor, as seen in the X-ray projection images. In some embodiments, the sensor (which can be a probe, a catheter, a guidewire) is segmented in the 2-D X-ray images using any suitable 2-D segmentation method. After the sensor is segmented, a virtual 6-DOF position of the X-ray source position and orientation is searched such that the 2-D re-projected 3-D shape of the tracked sensor will match the 2-D segmented sensor in the X-ray image, using suitable image matching and/or curve matching techniques. When the sensor's shape is non-trivial (for example, not a straight line) there will only be a unique solution which matches between the 2-D re-projected 3-D shape of the tracked sensor and the 2-D segmented curve of the sensor in the X-ray image. This match will belong to the X-ray source's true 6-DOF position and orientation in space, which will help in solving the X-ray's position during procedure, without additional fiducial markers, only based on the sensor's EM tracked 3-D shape.

The operations of blocks 122-128 are preferably repeated in a loop during a procedure in which a probe sensor is navigated. At any given time, a current deformed state of the model produced at block 128 may be available for use in other operations, e.g., those of box 130. There is no particular requirement that operations of different types are synchronous (that is, proceeding in strictly synchronized time order). For example, operations of box 130 may proceed using a first state of the deformed 3-D model while operations of box 120 are simultaneously performing calculations to update it to a second state.

Within box 130, in some embodiments, the shape and location of the curving probe (that is, the endoluminal device) are used in preparing one or more navigation displays. For purposes of describing their use in navigation display, the deformed model together with the endoluminal device are referred to as an updated 3-D “map”.

Block 131 generally represents display of the endoluminal device, its shape, or some portion thereof in situ with respect to the deformed 3-D model of the vasculature (or other luminal structure). For example, there may simply be a display of all or nearly all of the endovascular device and the luminal structure it occupies, together within a common coordinate space, optionally projected to a 2-D space for flat screen display, and/or represented in 3-D, e.g., using a virtual reality and/or augmented reality display.

Other blocks indicate features of which may be available to apply to the view of block 131, and/or may be applied to views of their own. They are optionally activated or provided together, and/or according to view selection states which may be actives by a device operator, and/or triggered automatically according to events such as reaching or passing bifurcations and/or other navigational waypoints or obstacles.

At block 132, in some embodiments, an optional map display (e.g., one like that described for block 131) is adjusted to emphasize endoluminal device tip visualization (e.g., “optimized”, according to implementation-dependent criteria that define what this means). Adjustment comprises, for example, zooming (magnification) of the map so that details of the tip with respect to the surrounding luminal structure are easily discerned. The tip may be brought to the foreground of the 3-D space in which it is represented, and/or intervening features between it and the viewer position may be suppressed in whole or in part. In some embodiments, the map is rotated so that the device tip is viewed in a consistent orientation for a certain type of operation (e.g., advancing from the left in preparation to navigate a bifurcation, or from another direction as may be selected by user preference and/or implementation details). Optionally, care is taken to select this orientation so as to maintain consistency with the operator's sense of physical space, e.g., with respect to the patient body, and/or with respect to the directions of motions which they carry out to steer the endoluminal device.

Additionally or alternatively, for example during operations where the device tip is approaching a bifurcation, the map may be rotated so that the bifurcation options are widely separated in the view provided—e.g., the plane presented as parallel with the plane of a 2-D display is a plane which crosses through the bifurcation junction, and a point some distance along either bifurcation choice. Optionally, the segment on the side of the direction of approach is considered, e.g., the plane crosses preferentially through a point on this segment away from the bifurcation junction itself. Optionally, a plane orientation is selected according to a weighting of the orientations of the segments and their junction.

Optionally, additional indications are provided which assist in navigation, particularly where a junction selection is about to be made. For example, the branch of a junction which appears to be currently selected if the endoluminal device is advanced from its current configuration is optionally highlighted or otherwise marked. If selection is unclear, this may be indicated by the absence of a mark/highlighting, or by a different indication of the present uncertainty. Optionally, available ranges of adjustment (e.g., steering curvature) which select one or the other of the junctions are indicated, e.g., by marks extending radially away from a position along the representation of the endoluminal device. Sequences of steering adjustments that potentially help to navigate a junction or other structural feature such as a blockage or tight turn may be indicated in turn as critical points in navigation are passed.

Other recommended adjustments may be provided based on recently acquired data. For example, there may be a preference to select in each moment the most “slippery” motion. In some embodiments, for example, small manual or automated trial motions are attempted, each receiving rapid feedback from along the probe's longitudinal extent regarding its shape, its position, and optionally also comprising data received from other sensors (e.g., strain sensors also provided as part of the device being navigated). Motions with the best recently recorded performance may be noted and optionally recommended (e.g., on a display). In automatic embodiments, successful movements may be executed, amplified, and/or repeated, and the process of testing repeated.

Optionally, automatic and manual control is combined. For example, automatic control may tend toward placing the device in a calculated best known state for advancing (e.g., combining commanded biases to steering angle, torsion and/or longitudinally exerted force). Manual commands superimpose on this state, potentially simplifying an operator's search for a successful combination of control inputs to pass through a present section, obstacle, and/or waypoint.

Tracking the shape of a probe is also potentially advantageous for driving an endovascular, or more generally, endoluminal probe, whether manually or robotically. Using the probe's tracked shape as feedback, the probe's steering can be made more efficient in an attempt to drive the probe from an origin to some destination. For example, when pushing the probe, the probe's shape may tell how the push action affected the probe and whether the probe did advance in response to the driving action, or whether, for example, a loop has formed along the shape of the probe, in which case the probe may need to be pulled back. This kind of visual shape feedback, along with others, can be used manually by a operator holding the probe and manipulating it (for example, a guidewire), or by an operator holding a remote controller for a robotic driving mechanism which drives the probe, or for a semi-automatic or a fully-automatic robotic driving mechanism, which may use the probes real-time tracked shape, as the operator would, to realize how steering actions are acting on the final shape of the manipulated probe.

Optionally, curvature of the device away from the plane of display (and accordingly, in directions along which position is naturally suppressed by the choice of perspective) is indicated by an assisting mark. For example, a different coloration (e.g., red or green) may be superimposed on probe areas depending on whether they bend toward or away from a view point perpendicularly in front of the display. Other types of indication for this condition optionally include simulated focus blurring, simulated shadows, warning symbols.

In some embodiments of the present disclosure, a tracked shape sensor is used in a standard roadmap-based workflow. In a standard roadmap-based workflow, contrast is applied to the blood vessels to draw a momentary “roadmap” of the blood vessels at the proximity of a catheter, as can be seen in live X-ray images capturing the spread of the contrast until it vanishes in the blood flow. By replaying the contrast injection in the acquired X-ray images (which can be acquired, for example, from 2 different X-ray views), a 2-D roadmap (or sometimes 3-D reconstruct roadmap) is saved and is statically subtracted from further acquired X-ray images, to serve as a baseline for navigation. The vessels, as enhanced by the contrast, are seen in 2-D and the operator then tries to steer a radiopaque device (for example, a guidewire) based on the subtracted roadmap towards a certain specific target in the anatomy. To get a feedback for navigation, the operator uses further X-ray images to get a 2-D tracking of the navigated device, on top of the subtracted roadmap. However, according to some embodiments, with a 3-D shape tracked device, once a roadmap is acquired (for example, as described above), no further X-ray images are needed in order to navigate the device on top of the roadmap. The device's 3-D shape is tracked in real-time and can be re-projected on the acquired 2-D roadmap using EM to X-ray registration methods, as described above. The device's re-projected 2-D shape can then be displayed (augmented) on top of the previously acquired roadmap, exactly as it would be seen by further X-ray images. Using this X-ray and EM combined method, the navigation steps in-between roadmap creation are spared and the X-ray dose drops significantly for these navigational procedures. Using the re-projected 2-D curve of the 3-D shape tracked device also has the advantage over X-ray imaging, in which the device's full curve may not always be clearly seen in the 2-D X-ray images due to various causes.

In some embodiments, using the re-projected 2-D curve of the 3-D shape tracked device also has the potential advantage over X-ray imaging in that it may potentially be tracked in a frame-rate higher than X-ray imaging. For example, at a frame-rate above 60 Hz, or above 30 Hz, to provide much faster feedback to the operator (which can be a human physician, a robotic driving mechanism or other).

In some embodiments, Using the re-projected 2-D curve of the 3-D shape tracked device also has the potential advantage over X-ray imaging in that its display can be enhanced by 3D information. For example, depth information, which is absent in standard 2-D views, but is present with 3-D shape tracking, can be incorporated into the 2-D view. For example, by using color which indicates depth along each point of the tracked device, and/or by a deliberate depth-dependent blue effect to imitate Depth of Field (DoF), and/or by creating two stereoscopic views which can be viewed with special 3D glasses and which give the sense of depth to a human viewer.

In another embodiment, in the case of a 3-D reconstructed angiogram (3-D roadmap), the 3-D roadmap can be re-projected to create one or more 2-D views, as could be seen by additional X-ray image acquisitions. Since the roadmap is now tridimensional, it can be rotated virtually to create views from any desired angle (not just a single or a fixed number of predetermined 2-D angles). The operator can then be presented with one or more 2-D virtual views, which correspond to standard 2-D subtracted roadmaps, on which navigation can be performed with a device's 2-D re-projected 3-D tracked curve. Additionally or alternatively, according to some embodiments, the operator can be presented with a 3-D view of the 3-D roadmap, and a 3D display of the device's tracked shape in a virtual 3D view, which can be rotated such that the operator is able to understand the 3-D state of the navigated device inside the vessels.

In some embodiments, the 3-D re-projected roadmap can also be displayed with 3-D enhanced effects, such as Depth of Field, stereoscopic views, etc., as mentioned above.

In some embodiments, the 3-D shape tracked device can be displayed in its position on top of the 3-D roadmap, in a true 3-D view, with a static, automatic, rotating or controllable virtual camera, to present the operator with the full 3-D information, instead of re-projected 2-D views.

In some embodiments, one or more EM reference sensors can be attached, for example, to the patient's cranium, to track the potential movements of the patient during procedure. In a standard X-ray roadmap-based navigation, if the patient moves then the roadmap may no longer be registered to the newly taken X-ray images. In the newly taken images, the radiopaque device (such as a probe, a catheter, a guidewire or other) would move in position together with the movement of the patient, while the roadmap would stay in place (as it is usually a static subtracted image). This creates a mismatch between the live projected curve of the device, as seen in the live X-ray images, and the offline roadmap image. In some embodiments, this could lead to difficulties in the navigation of the device to a desired target. In some embodiments, with EM reference sensors attached to the patient, the movements of the patient can be tracked and accounted for as to maintain the registration between the patient (in EM coordinates) and the X-ray images. According to some embodiments, in a roadmap-EM fused view, the curve of the device is re-projected on the offline fluoroscopic image, as described above, and used for navigating on the offline roadmap without the need for additional X-ray imaging. When the patient moves, EM reference sensors (for example, 6-DOF point sensors), which are attached to the patient (for example, to the cranium of the patient), can track this movement. Then, a compensation transformation between the pose of the patient (for example, the patient's cranium) is computed, from when the roadmap was taken, and the current real-time tracked pose of the patient (for example, the patient's cranium, as being tracked by the reference sensors). This compensation transformation can then be applied on the tracked curve of the device before re-projecting the device on the offline roadmap image, such that the re-projected curve of the device maintains its registration to the offline roadmap image. This enables accurate navigation of the endoluminal device on the 2-D/3-D roadmap even in cases where the patient moves. Additionally or alternatively, when the patient moves (as tracked by the EM reference sensors), a 3-D roadmap can be moved in 3-D and then re-projected as a 2-D view, so that the 2-D displayed roadmap will update according to patient's movements (instead of being static, as with other standard solutions).

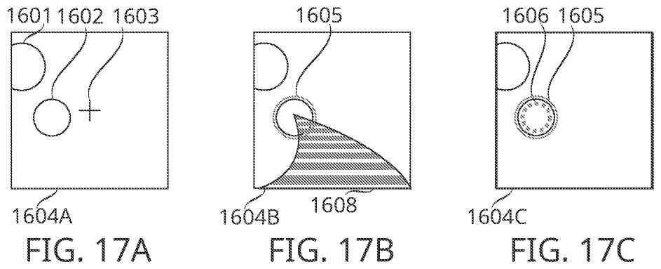

At block 134, in some embodiments, an optional display view is presented (additionally to other views, and/or switched to) which represents a “first person” point of view from within the space of the luminal structure. For example, the view may be as if looking distally from a distal tip of the endoluminal device; or a little removed from it, e.g., a “following” or “riding” view. In following or riding views a portion of the device may be shown and/or indicated in a literal representation. Optionally, representation of the device is abstract, for example as described in relation to FIGS. 17A-17C. The view is optionally oriented rotationally to be consistent, e.g., with respect to a particular steering axis, with respect to the orientation of bifurcation branches about to be traversed, or with respect to a weighted combination of both. Indications may be provided to indicate steering ranges, selections, and/or sequences, for example as described in relation to block 132, and/or FIGS. 17A-17C

At block 136, in some embodiments, an optional display view is presented (additionally to other views, and/or switched to) which represents a “roadmap” view of the current procedure which incorporates recently acquired procedure imaging results together with the 3-D map.

The imaging results are optionally being acquired live, e.g., updating automatically during the procedure itself. Updating is not necessarily continuous or at a high frequency. In some embodiments, fluoroscopic images of vasculature acquired during a procedure document the current position of the endovascular device, which may be radiopaque in whole or in part. Other details may also appear; for example, an injection of contrast agent may transiently highlight an approaching bifurcation. Implanted devices may be visible, and elements placed as and/or acting as fiducial marks may be visible.

There are at least two sources of information which may be used together or individually to help align the 3-D map (e.g., as described for block 131) with these new imaging results. First, insofar as the 3-D shape of the endovascular device is visible (as a 2-D projection) in one or more of the newly acquired imaging results, its 3-D shape at the moment of data acquisition may be used to determine how the model should be aligned so that a consistent 2-D projection of the modeled device is produced (e.g., so that a 2-D “shadow” of the device matches up with its appearance in the image). Second, if the model originally generated at block 118 used the same angiographic imaging system with the patient in the same position, the coordinate system associated with the new image is the same as that of the (un-deformed) model. Moreover, if deformation estimates are correct, then the map, viewed from an appropriate angle, should match the angiographic image. If not, then features which appear in both map and angiographic image can be used to recalibrate. In any case, with sufficiently frequent updates, divergences potentially become immediately apparent.

Additionally or alternatively, according to some embodiments, alignment between the 3-D map and the new imaging results is achieved using registration between the EM coordinates and the X-ray source. This registration can be achieved using the methods described above.

Optionally, the angiographic image is distorted as necessary to match the map, and/or the map is distorted to approach the geometry of the angiographic image. There may be pulsations in the shapes imaged by the angiographic image, e.g., due to physiological motions such as respiration and heartbeat. These may be minimized by suitable gating, e.g., to a particular phase of cyclical physiological motions. Optionally, measured motions of the endoluminal device are indicative of the pulsing (or a least, indicative of pulsation in locations relevant to the task of navigation). Cyclic motions may be compensated for in the display of the map by isolating the repeating component of device motion (e.g., within cycles having periods corresponding to the heart rate and/or respiration), and then injecting contrary motions into the displayed map. The same cyclic corrections may be applied to the angiographic image, albeit with some estimation needed for image regions away from the device. Representation in the depth axis of the image may also be somewhat distorted away from the device itself (e.g., for motions of structures crossing over the device), but this potentially has little impact on navigation tasks concerned with events near the device in any case.

Additionally or alternatively, cyclic motions may be compensated by using measurements of reference sensors, which are attached to the patient. For example, respiration and heartbeat may be sensed and measured by the reference sensors, either in their motion (as being tracked by the EM tracking system) or by additional special sensors incorporated inside, such as accelerometer, gyroscope, ECG or others. By sensing the cyclic motions using the reference sensors, their effect on the tracked device can be more easily compensated since it is then possible to separate between the cyclic motion (the phase and amplitude of which may be sensed by the reference sensors) and the true motion of the shape tracked device, as tracked by the EM tracking system.

If it becomes apparent that common features of angiograms and map such as the endoluminal device “shadow” are sufficiently out of registration, then optionally the operations of box 110 are repeated using new images of at least a portion of the vasculature to help restore calibration. Additionally or alternatively, calibrations used at block 124 to reconstruct the 3-D catheter shape are adjusted.

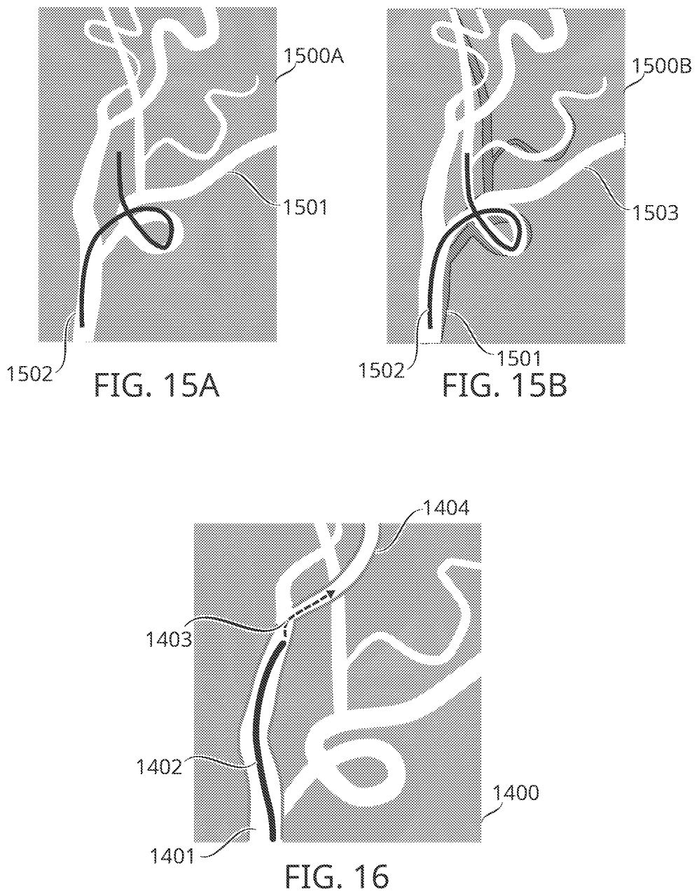

With both views aligned, the view of block 136 may be set to selectively mask the angiographic view, and/or selective add overlay features known from the map. For example, a targeted path for the angiographic device may be marked by a center line and/or vascular outline. Areas away from this path may be digitally masked to suppress them, e.g., masked to suppress distracting misalignments which are irrelevant to the current procedure and/or navigational task. Optionally, the angiographic image is marked to indicate navigational information such as the selected path, available range (and/or likely effects) of navigational steering, out-of-plane aspects of the device position described in relation to block 132. Features of view like that of block 136 are also described with respect to FIGS. 15A-15B and FIG. 16.

Reference is now made to FIGS. 15A-15B, which schematically represent an acquired visualization of a lumen anatomy 1502, according to some embodiment of the present disclosure.

The visualization 1500A of lumen anatomy 1501 presents, for example, an angiogram of arteries (e.g., cerebral arteries), into which a guidewire 1502 is introduced. In FIG. 15A, it is shown that the sensed curvature and position of guidewire 1502 appears to be floating outside the lumen of lumen anatomy 1501. In FIG. 15B, the visualization (in this case, the angiogram) is deformed such that lumen structure 1503 is adjusted to the visualization of lumen anatomy 1500B. For reference, the pre-deformed lumen anatomy 1501 is also depicted in FIG. 15B in its original position and shape.

To perform the registration, guidewire 1502 is used as the ground-truth of the “in vivo” position of the anatomical structures shown in 1500A, for example as described in relation to block 136 of FIG. 14B. The adjustment depicts at least the reality that guidewire 1502 is inside the lumen, although there may be associated geometrical distortions introduced that are not strictly “real” in the sense of presenting an undistorted view of all spatial arrangements. In some embodiments, the curve position of the guidewire may be tracked in 2-D using the fluoroscopic images and/or can be tracked in 3-D using a 3-D shape tracked guidewire, as described above. In some embodiments, it is then re-projected to the 2-D fluoroscopic images using EM to X-ray registration methods, as described above. In some embodiments, alternatively, the curve position of the guidewire can be tracked using a combination of EM tracking and X-ray imaging.

Insofar as what matters for navigation is relative positions of guidewire 1502 (or another endoluminal device) and the vasculature (or other luminal structure) it occupies, a tradeoff in distortions away from the endoluminal device is potentially worthwhile. Areas away from guidewire 1502 are optionally distorted using conservative extrapolation; for example, enough to maintain continuity with areas where ground truth is available, but in a way that reduces with distance, and/or preserves original shapes.

Reference is now made to FIG. 16, which schematically represents an angiogram-like navigation screen 1400 using a 3-D deformed model of luminal anatomy, according to some embodiment of the present disclosure. The luminal anatomy comprises, for example, blood vessels.

Representation of the vessels 1401 may derive from the intensity values of one or more actual angiograms, optionally distorted, e.g., as described in relation to FIGS. 15A-15B. Additionally or alternatively, representation of guidewire 1402 (or another endoluminal device) in the navigation screen display is distorted to match its angiogram appearance. In some embodiments, the angiogram is shown entirely undistorted, including even the appearance of guidewire 1402 in the image, and other indications added artificially are deformed using the map-to-angiogram transform so that they appear in the correct places.

Alternatively, in some embodiments, representations of anatomical features and the guidewire 1402 itself are largely synthesized. They are optionally “made up” to look like angiographically revealed vessels, in whole or in part. Optionally, a region of angiogram image intensity data near the guidewire 1402 (i.e., where registration is based on direct feature comparisons) is preserved. This can be helpful, e.g., to confirm the local environment of navigation, and/or to maintain the capability of visualizing the real impact of manipulations such as contrast injection to reveal local features.

Optionally, navigation images are presented in manner that uses more “original” angiogram grayscale data soon after they are acquired, but replaces this with synthesized data, optionally adjusted to match angiographic appearances, as the image ages, until a new image is taken. This may promote anchoring to the real navigational situation (in the form of navigational images that show full context), coupled with rapid feedback on navigational actions, while potentially reducing a need to expose the patient and/or operator to imaging radiation.

Optionally, there is a brief (e.g., 100 msec or less) period of reverse blending to “fade in” new image data when it becomes available, which potentially reduces jarring jumps that interfere with the sense of navigation.

Imaging condition manipulation may be used in conjunction with some embodiments of the present disclosure. As an example: the procedure of contrast agent injection in a vasculature predictably darkens vascular regions progressively as the dye is carried along by the circulation. Optionally, image data from a plurality of angiographic images are combined to show the “darkest recent pixel value”, at least in some region near (e.g., including) the probe. Display of this result may be triggered by a dye injection event, and may persist as long as is found useful, e.g., until cleared by an operator command, and/or limited by a certain period length. Optionally (e.g., to reduce image merging artifacts), contrast agent darkening from a single image is shown for a region near the advancing tip of the guidewire 1401, and replaced with another image when the tip advance to a new location. Thus, the angiographic image data shown optionally do not necessarily reflect the most recent image taken, but may rather preferentially display image data which is informative because it shows a particularly clear view of the region in which a tip of an endoluminal device (e.g., guidewire 1401) is presently operating.

In some embodiments, image information which is spatially anchored to the coordinates (e.g., pixel positions) of angiographic images in which it appears is algorithmically extracted (e.g., by thresholding, difference analysis, or another method) and converted to a synthetic appearance (e.g., an outline, blob, or other element). The synthetic appearance retains its association with the image locations the source data was originally anchored to, so it can be shown in place of the original data. This provides potentially greater control to maintain a constant appearance during updating, since the switch-over between angiographically determined data and shape-sensing provided data is in either case mediated by wholly synthesized image indications. A more constant appearance potentially helps avoid disrupting an operator's sense of device operating conditions and/or device responses to operating inputs.

In some embodiments, arrow 1403 and/or vascular outlines 1404 mark a projected representation of an algorithmically predicted (most probable) 3-D curved path the guidewire 1401 will take if pushed forward from its present position. In some embodiments, vascular outlines 1404 are provided as a targeted pathway indication, e.g., in accordance with a pre-planned path or computed best path to target.

A mark may be indicated on the screen 1404 as a continuation of the guidewire shape (e.g., continuation through arrow 1403). Optionally, these marks are updated continuously as guidewire 1402 (or another shape-tracked endoluminal device) is moved, bent, or torqued; and as the anatomy is deformed, such that it always displays the probable path the guidewire is predicted to take.

In some embodiments, a predicted curve of the probe can be drawn on screen based on an estimation of the curve of the probe after being pushed or manipulated in any specific way. In some embodiments, this provides the operator with information of how the probe would be curved, bent, and/or positioned (the guidewire's “future” state) after being pushed or generally manipulated. In some embodiments, it provides a predictive view (“a futuristic view”) of the future state of the probe and can aid the operator in taking steering decisions throughout the navigation (for example, prefer certain maneuvers to others).

In some embodiments, in order to predict the future state of the probe, the current state of the probe, that is, the real-time EM tracked 3-D position in space is used in its tracked position inside the 3-D map (which can be deformed). In some embodiments, then, a motion model is used to predict the future state of the probe under certain hypothetical steering actions (such as push/pull/rotate etc.). According to some embodiments, the motion model can be based on a 3-D physical simulation software, for example, a finite-element simulation, which takes into account the flexibility of the navigated probe, as well as the flexibility of the vessels in the 3-D map with or without a supporting deformation model. The simulation is then able to virtually “push” the physically simulated device (as it is posed at a certain moment in time, based on its tracked curve inside the 3-D map) and simulate its predicted motion with respect to the steering action inside the deformable 3-D map, based on the physical simulation. The final rest state of the probe can then be presented to the operator during navigation, and/or its complete motion sequence can be displayed in a short “predictive video”, for example, played in a loop, to show where the system has calculated the location where the probe would move had it been pushed.

In another embodiment, a neural network can be used to predict where a probe would move, based on its current tracked 3-D curve inside the 3-D map. In some embodiments, to achieve that, a neural network, for example a deep neural network such as a U-Net, can be trained to receive a 3-D multi-channel image of the 3-D vessel map along with a 3-D image of the current probe tracked curve. Then, the system outputs a 3-D image of the predicted probe curve inside the same 3-D map, had it been pushed. In some embodiments, to train and test this neural network, a set of inputs and outputs can be collected from real procedures and/or from physically simulated procedures. For example, where a probe is placed at a certain pose marked as “present pose” and when the probe actually moves, as tracked by the EM tracking system, its resulting new pose is recorded as its “future pose”. The neural network can then be fed with “present pose” and “future pose” counterparts, both represented for example as 3D images inputted to the neural network, which can be for example a 3-D CNN U-Net as described above, and the neural network is instructed to output “future pose” for “input pose”, for a dataset of collected such pairs. Similarly, a physical simulation software can be trained using a similar method, according to some embodiments, by using “present pose” states to predict “future pose” states, and comparing the simulator's output with the truly recorded “future states”, as were recorded by the EM tracking system.

By using the 3-D tracked curve of the shape-tracked device, prediction of motion becomes possible, based on the 3-D tracked curve and based on trained physical simulations or predictive neural networks.

Indications may change appearance (e.g., strengthen/fade, lengthen/shorten, change size otherwise, and/or change color) according to the probability confidence. Options for control may be presented, particularly under conditions where successful navigation may comprise a plurality of commands in sequence, e.g., an initially week steering command growing stronger, an initially strong steering command growing weaker, or even a reversal of steering direction, e.g., to moderate force exerted on the far side of a waypoint or obstacle after a tip successfully traverses it.

Optionally, variations in the indication sequences themselves are provided to help coach complicated operations of the device to pass an obstacle and/or waypoint. For example, there may be defined within a repertoire of the system indication sequences which help coach application of a maneuver sequence which is intentionally attempted a plurality of times with slight variations in order to successfully pass an obstacle and/or waypoint. The indications may coach, for example, cessation of a particular attempt to advance, return to a starting point for another attempt, and/or incremental adjustment of the device state in preparation for another attempt. Optionally, there are indications for the advancing maneuver sequence itself; e.g., a degree, position, and/or timing of torsion (twist around a proximal-distal axis) and/or steering (bending away from the proximal-distal axis) during advance along the proximal-distal axis.

In selecting indications to show, the device's own properties are preferably considered; for example, its flexibility and/or geometry (e.g., its diameter). The length of the endoluminal device which is being pushed behind its advancing tip is optionally considered. In some embodiments, observational data are used, e.g., data gathered during tests and/or prior procedures using the device. The observational data may be converted into a heuristic rule. A heuristic rule describes, for example: an observed parameter threshold above or below which the indication is considered applicable; an observed parameter range over which the indication is modified in one or more of its parameters; and/or another function relating states of known, estimated, and/or observed parameters to associated observed results). In some embodiments, the observational data are used to guide the training of a machine learning algorithm. The algorithm produces a machine learning product that can receive inputs describing aspects of a current device and/or environment state, and provide output that the system uses in determining how indications are displayed. Optionally, the machine learning product is pre-programmed into the system.

Optionally, the machine learning product is analyzed, and converted into one or more heuristic rules. Optionally, the machine learning training set includes data gathered from the current site, operator and/or procedure. The machine learning product is then customized and/or dynamic, potentially resulting likewise in customized/dynamic conditions that trigger/modulate indication presentation.

Optionally, input/output behavior of the modified machine learning product is automatically probed to gain information that is then used to adjust heuristic rules of the system so that they suit conditions of the particular site, operator and/or procedure. Optionally, parameters of heuristic rules are customizable manually. For example, a user can raise or lower the sensitivity of the system to conditions that produce a certain indication.

While customization has been described under the categories of customization per site/operator/procedure, it should be understood that there may be underlying differences in another relevant aspect of a procedure using an endoluminal device according to some embodiments of the present disclosure. These differences may be what produce customization differences in what indications are meaningful. Examples include: the make-up of a site's particular patient population, procedure style of an operator, and/or the disease state of a particular patient. Customization and/or adjustment of indication presentation based on any such factor (e.g., machine learning using the factor as an input), whether manually or automatically implemented, is also contemplated for some embodiments of the present disclosure.

Any of the indications (e.g., provided as guidance/coaching and/or as indications of current or predicted state) is optionally triggered and/or modulated in response to sensed data. The sensed data may be data provided using sensing of the device's own state and/or effects. This may in particular include the shape and direction of the tip of the endoluminal device. Optionally, state parameter are considered in combination. For example, cessation of advance may be indicated (coached) when certain shape and/or position conditions are sensed in conjunction with an attempt to advance. Examples of these conditions include: increasing device curvature, failure of the tip to actually advance, or advancing of the tip while the device tip is in contact (e.g., within a certain range of contact angles) upon its distal end with a luminal wall. Optionally or alternatively, imaging results (e.g., of tissue deformation), impedance sensing (e.g., of tissue contact), and/or sensing of strain/stress by auxiliary sensors of the device is used by the system to help determine how indications are shown.

Additionally or alternatively, in some embodiments, algorithms governing indication display take into account factors of the device environment, sensed or otherwise known or estimated. Such factors optionally include, for example, the direction of blood flow, and the diameter of the navigated vessel.

In embodiments using machine learning, any of the sensed and/or otherwise known data and/or factors are optionally used as inputs to the machine learning algorithm.

Reference is now made to FIGS. 17A-17C, which schematically represent “first-person” views of navigation in a 3-D model, wherein the view represents what lies in front of the tip of an endoluminal device, according to some embodiment of the present disclosure.

In view 1604A of FIG. 17A, a cross-hair 1603 indicates the direction the tip is directed towards, with bifurcation openings 1602, 1602 visible. To enter one of the openings, the operator manipulates the endoluminal device (guidewire) such that the cross-hair 1603 points at (overlies) the desired opening, and then pushes the endoluminal device (e.g., a guidewire) forward. In some embodiments, cross-hair 1603 indicates an estimated current orientation of the endoluminal device, so that it is not necessarily expected to align with the target until the device approaches more closely. The operator in this case may use observations of the angle and/or speed of relative approach of cross-hair and target during movements along a proximal-distal axis of the device in order to judge device adjustment. Additionally or alternatively, in some embodiments, a cross-hair 1603 is provided which overlies a region toward which the device tip is expected to advance, for example as also described in relation to FIG. 17B. For example, based on predictive physical simulations of the tracked device or on a predictive neural network as described above, or using any other suitable prediction method.

View 1604B of FIG. 17B is a schematic illustration of an embodiment wherein the targeted bifurcation opening is marked by highlighted 1605. A probable trajectory curve that the endoluminal device (guidewire) is predicted to take is indicated, e.g., as a curving 3-D “rail” 1608. It is updated continuously as the guidewire is moved, bended or torqued, and/or as the anatomy is deformed, such that it always displays the probable path the guidewire is predicted to take. The path does not necessarily converge to a point as shown. For example, it may be wide to take into account uncertainty in the prediction of its trajectory. It may also incorporate a height indication indicating vertical uncertainty, e.g., near its terminus, such as a circle around the end of the path, sitting atop it, or otherwise positioned. Additionally or alternatively, the path indication may be provided with width and/or height to account for the diameter of the endoluminal device tip. The path indication may terminate centered on the projected center of the endoluminal device tip, or offset from it, e.g., below it, as if the device is sliding along and over the indicated path.

The pathway view has the potential advantage of indicating a potentially successful future orientation that may be different from a current device tip orientation (but is still “implied” by it), along with that current orientation. Furthermore, it provides an indication of how movement is expected to occur along that path. This may assist in judging how safe or realistic the prediction is (e.g., considering other factors that the operator may be aware of such as occlusive obstructions visible in an angiographic image).

In some embodiments, the curve predictions of the endoluminal device (guidewire) are based on the device shape tracking, without the need for X-ray imaging to track the current curve of the device and to predict the future curve of the device, as described above.

In some embodiments, an aspect of the path's presentation is adjusted to reflect certainty of reaching the target. The adjustment may indicate different certainty with respect to different positions. For example, it may have faded contrast nearer to the current location (e.g., nearer to the bottom), but stronger contrast in regions nearer to the target in perspective (e.g., higher up/nearer to the path's end). In some embodiments, an aspect of the path's presentation is adjusted to communicate the size and position of an estimated “funnel” of tip positions from which further advance is predicted to lead to the target. This may be provided, for example, as the visible width of the path, which accordingly, in particular, may bend or narrow to avoid non-targeted bifurcations.

In some embodiments, the tracked curve and predicted curve of the device can be used to drive the device robotically, whether manually, semi-automatically or fully-automatically. For example, the tracked curve of the device can be used to provide feedback for the robotic steering of the device, for example, to avoid loops in the curve of the device while steering or pushing the device towards a certain desired location. In some embodiments, this is done without the need for X-ray images. In some embodiments, the predicted curve of the device can also be used for the robotic drive, for example, by torqueing, the device until the predicted curve aligns with the pathway to the target, and once aligned, only then the device is pushed towards the target. In some embodiments, this maximizes the probability to advance towards a desired target.

View 1605C of FIG. 17C is a schematic illustration of an embodiment where again the desired opening is highlighted 1605. The probable opening the guidewire is predicted to enter (in its current orientation) is indicated by additional markings 1606. To navigate, the operator torques the guidewire until the correct opening is highlighted by the additional markings, and then pushes the endoluminal device (e.g., guidewire) forward. In some embodiments, as discussed above, the operator can be replaced and/or complemented with a robotic driving mechanism (either fully automated, or semi-automated—and optionally AI-based), which replicates the operator's behavior in searching for the, optionally, most effective steering action to push the device towards a desired target.

In general, any guidance indication (e.g., as shown in any of FIGS. 17A-17C, and/or as described in relation to displays of FIG. 14B and/or FIGS. 15A-15C) may be varied in one or more presentational aspects (e.g., color, shape, brightness, saturation, size, contrast, texture, and/or position) to indicate a corresponding aspect of the indication's meaning. The varied presentational aspect may itself include one or more temporal components, e.g., a frequency, abruptness, and/or duty cycle of a cyclical change in one of the just listed presentation aspects, and/or a one-time event such as a flash.

A “corresponding aspect of indication meaning” may include the indication's basic semantic message—e.g., to show where or what the target is, and/or whether or not navigation is on target. Optionally, the “corresponding aspect of indication meaning” is a measure of confidence that the basic semantic message shown is also correctly assessed—e.g., an indication may be de-emphasized and/or “smeared” in some fashion if the confidence measure is low. Optionally, the “corresponding aspect of meaning” is a measure of probability that a situation implied by the basic semantic meaning will actually happen—e.g., again, an indication may be de-emphasized or “smeared” in some fashion if the assessed probability is low.

A clear difference between “confidence” and “probability” is not always present; but where different, the two may be distinguished by considering “probability” as the assessed likelihood that something will happen (e.g., 80% chance of reaching a target), and confidence being a measure of the assessed likelihood itself (e.g., a probability of 80% may be the middle of a confidence range of 70%-90%, or merely one value of a wider (and therefore “lowered”) confidence range of 20%-100%). This relationship could be, for example, that between a statistically calculated mean probability, and its associated error range (confidence).

A corresponding aspect of meaning may be of another type. For example, presentational aspects may be used to indicate temporal windows (e.g., for a moving target, such as in a beating heart), cautions and/or warnings (e.g., when a target is potentially near or below the diameter of the probe tip, or otherwise vulnerable to damage), and/or difficulty (e.g., based on an assessment that a particular traversal may call for special care).

### General

It is expected that during the life of a patent maturing from this application many relevant variably resistive, variably inductive, and/or high-permeability materials will be developed; the scope of the term variably resistive material, variably inductive material, and/or high-permeability material is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, to the same extent as if each individual publication, patent or patent application was specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

1. A method of displaying a navigational view for an endoluminal device, comprising:
a. receiving at least one 2-D image comprising a lumen to be navigated by said endoluminal device;
b. detecting a 3-D location of a tip and a shape of said endoluminal device;
c. calculating a position and/or a shape of said endoluminal device within said at least one 2-D image;
d. displaying on said 2-D image said endoluminal device according to said calculation;
e. amending said displaying of said endoluminal device on said 2-D image by repeating steps “b” and “c” while said endoluminal device is being moved.

2. The method according to claim 1, wherein said method does not require retaking new images in order to perform said “e”.

3. The method according to claim 1, wherein said at least one 2-D image is a 2-D X-ray image.

4. The method according to claim 1, wherein said at least one 2-D image comprises a 2-D view of at least one segment of said endoluminal device.

5. The method according to claim 4, wherein said calculating a position and/or a shape of said endoluminal device comprises comparing said at least one segment as viewed in said at least one 2-D image with said detected 3-D location of said tip and said shape of said endoluminal device in order to identify a location of said at least one segment along said endoluminal device.

6. The method according to claim 5, further comprising utilizing said identified location to perform a comparison between said detected 3-D location of said tip and said shape of said endoluminal device and said at least one 2-D image.

7. The method according to claim 6, further comprising analyzing said comparison to generate a navigational view of said endoluminal device on said at least one 2-D image.

8. The method according to claim 7, further comprising displaying said generated navigational view by re-projecting a result of said analysis on said at least one 2-D image.

9. The method according to claim 8, further comprising amending said displaying of said generated navigational view by repeating:
a. said calculating a position and/or a shape of said endoluminal device comprises comparing said at least one segment as viewed in said at least one 2-D image with said detected 3-D location of said tip and said shape of said endoluminal device in order to identify a location of said at least one segment along said endoluminal device;
b. utilizing said identified location to perform a comparison between said detected 3-D location of said tip and said shape of said endoluminal device and said at least one 2-D image;
c. analyzing said comparison to generate a navigational view of said endoluminal device on said at least one 2-D image; and
d. displaying said generated navigational view by re-projecting a result of said analysis on said at least one 2-D image;
while said endoluminal device is being moved.

10. The method according to claim 9, wherein said amending does not require retaking new images in order to perform said amending.

11. The method according to claim 1, wherein said at least one 2-D image comprises, within said at least one 2-D image, a 2-D view of one or more EM markers and/or EM reference sensors located at EM known locations.

12. The method according to claim 11, further comprising one or more of:
a. correlating said known 3-D location of said one or more EM markers and/or EM reference sensors with a location of said one or more EM markers and/or EM reference sensors in said 2-D image;
b. comparing said correlation with said detected 3-D location of said tip and said shape of said endoluminal device;
c. analyzing said comparison to generate a navigational view of said endoluminal device on said at least one 2-D image;
d. displaying said generated navigational view by re-projecting a result of said analysis on said at least one 2-D image.

13-15. (canceled)

16. The method according to claim 1, wherein said at least one 2-D image comprises a 2-D view of a plurality of markers located in known locations along said endoluminal device.

17. The method according to claim 16, further comprising one or more of:
a. comparing a location of said plurality of markers located in known locations along said endoluminal device as viewed in said at least one 2-D image with their actual 3-D known location along said endoluminal device and according to said detected 3-D location of said tip and said shape of said endoluminal device;
b. analyzing said comparison to generate a navigational view of said endoluminal device on said at least one 2-D image;
c. displaying said generated navigational view by re-projecting a result of said analysis on said at least one 2-D image;
d. amending said displaying of said generated navigational view by repeating one or more of:
i. correlating said known 3-D location of said one or more EM markers and/or EM reference sensors with a location of said one or more EM markers and/or EM reference sensors in said 2-D image;
ii. comparing said correlation with said detected 3-D location of said tip and said shape of said endoluminal device;
iii. analyzing said comparison to generate a navigational view of said endoluminal device on said at least one 2-D image;
iv. displaying said generated navigational view by re-projecting a result of said analysis on said at least one 2-D image;
while said endoluminal device is being moved.

18-20. (canceled)

21. The method according to claim 17, wherein said amending does not require retaking new images in order to perform said amending.

22. The method according to claim 1, wherein said detecting a 3-D location of a tip and a shape of said endoluminal device is performed utilizing one or more of an EM curve inductive sensor and an EM curve resistive sensor.

23. The method according to claim 1, wherein said detecting a 3-D location of a tip and a shape of said endoluminal device is performed by one or more of EM tip sensing, multi-sensor EM shape sensing, fiber optic shape sensing, passive RF sensing, sensing detectable magnets, sensing ultrasound-detectable markers and fluoroscopic shape tracking.

24. The method according to claim 1, wherein said displaying comprises displaying on a 3-D roadmap said endoluminal device according to said calculation.

25. The method according to claim 24, further comprising amending said displaying of said endoluminal device on said 3-D roadmap by repeating steps “b” and “c” while said endoluminal device is being moved.

26. The method according to claim 11, further comprising utilizing said EM reference sensors to track a movement of a patient; and said method further comprises compensating for said movements performed by said patient by moving said detected 3-D location of said tip and said shape of said endoluminal device accordingly.

27-39. (canceled)

40. A method of displaying a navigational view for a field of view for an endoluminal device, comprising:
a. generating a volume from a plurality of images;
b. detecting tip and shape of said endoluminal device;
c. deforming said volume based on said detected tip and shape of said endoluminal device; and
d. displaying a navigational view using said deformed volume and said detected tip and shape of said endoluminal device.

41. The method according to claim 40, wherein said generating a volume from a plurality of images comprises one or more of:
a. receiving said plurality of images;
b. analyzing said plurality of images to detect one or more vessels within said plurality of images;
c. combining multiple phases of said detected one or more vessels into a single data structure comprising vessels of interest in said field of view;
d. combining results from “b” and “c” into a common 3-D space, thereby generating said volume.

42. The method according to claim 41, wherein said combining results from “b” and “c” into a common 3-D space, further comprises combining vascular segments with their associated 3-D spatial extends.

43. The method according to claim 40, wherein said plurality of images are one or more of angiograms images, X-ray images, Cone-beam images, CT images, MRI images.

44. The method according to claim 40, wherein said volume comprises one or more data comprising descriptions of paths along which vascular centerlines extend, descriptions of nodes at which paths join and/or bifurcate, and descriptions of vascular cross-sections along the paths.

45. The method according to claim 40, wherein said generating a volume from a plurality of images further comprises associating said generated volume with a deformation model.

46. The method according to claim 41, wherein said receiving said plurality of images is performed in real-time.

47. The method according to claim 40, wherein said detecting tip and shape of said endoluminal device comprises one or more of:
a. associating between said endoluminal device and sensor raw data received from one or more sensors in said endoluminal device;
b. reconstructing a 3-D shape of said endoluminal device based on said association; and
c. detecting a shape location of said endoluminal device based on a coordinate system.

48. The method according to claim 47, wherein said one or more sensors comprise one or more of a inductive EM sensor and a resistive EM sensor.

49. The method according to claim 47, wherein said deforming said volume based on said detected tip and shape of said endoluminal device comprises one or more of:
a. calculating said deforming based on constrains imposed by said reconstructing a 3-D shape of said endoluminal device based on said association and said detecting a shape location of said endoluminal device based on a coordinate system; and
b. calculating said deforming based on received images taken in real-time.

50. The method according to claim 40, wherein said displaying is performed on a 2-D X-ray image.

51. A system configured for displaying a navigational view for an endoluminal device, comprising:
a. an endoluminal device;
b. a sensor for detecting a 3-D location of a tip and a shape of said endoluminal device;
c. a user interface; and
d. a processor unit configured to:
i. receive at least one 2-D image comprising a lumen to be navigated by said endoluminal device;
ii. calculate a position and/or a shape of said endoluminal device within said at least one 2-D image, based on the detected 3-D location and shape of said device;
iii. display on said 2-D image said endoluminal device according to said calculation; and
iv. amend said displaying of said endoluminal device on said 2-D image by repeating steps “ii” and “iii” while said endoluminal device is being moved.

52. The system according to claim 51, wherein the processor unit is configured to:
a. generate a volume from a plurality of images;
b. deform said volume based on said detected tip and shape of said endoluminal device; and
c. display navigational view using said deformed volume and said detected tip and shape of said endoluminal device.
